(19) United States
(12) Patent Application Publication
(10) Pub. No.: US 2026/0309713 A1
(43) Pub. Date: Oct. 8, 2026

(54) COMBINATION THERAPY OF POLIO VIRUS RECEPTOR INHIBITORS AND FUCOSE

(71) Applicant: H. LEE MOFFITT CANCER CENTER AND RESEARCH INSTITUTE, INC., Tampa, FL (US)

(72) Inventor: Eric K. LE-LAU, Tampa, FL (US)

(21) Appl. No.: 19/482,904

(22) PCT Filed: May 10, 2024
(86) PCT No.: PCT/US2024/028898

§ 371 Date: Nov. 10, 2025

Related U.S. Application Data
(60) Provisional application No. 63/501,289, filed on May 10, 2023.

(51) Int. Cl. G01N 33/575; A61K 31/7004; A61K 40/11; A61K 40/15; A61K 40/31; C07K 16/28

(52) U.S. Cl. CPC G01N 33/57585; A61K 31/7004; A61K 40/11; A61K 40/15; A61K 40/31; C07K 16/2818; C07K 16/2827; G01N 33/5759; G01N 2333/70596; G01N 2440/38

(57) ABSTRACT

Disclosed are methods for detecting and treating cancers and, in particular, metastasis based on the fucosylation of polio virus receptor (PVR). Said methods can comprise the detection of the fucosylation of PVR; wherein an increase or the presence of fucosylated PVR indicates a cancer and/or metastasis. Also disclosed are methods of treating said cancer comprising administering to a subject an agent that inhibits PVR and an agent that increases fucosylation.

### I. CROSS-REFERENCE TO RELATED APPLICATIONS

This application claims the benefit of U.S. Provisional Application No. 63/501,289, filed on May 10, 2023, which is incorporated herein by reference in its entirety.

### II. BACKGROUND

Breast cancer is still the leading cancer in women and exhibits significant brain metastasis rates particularly in triple negative breast cancer. Once the breast cancer metastasizes to the brain, the outlook for these patients drops significantly. Despite reports of striking efficacy, durable responses of immunotherapies have been limited to subsets of patients. In attempt to improve responses, clinical trials have tested combinations of immunotherapies with other therapeutic interventions, with limited success. Unfortunately, patients often experience significant adverse events, sometimes resulting in their withdrawal from the clinical trial. Another ongoing challenge with immunotherapies is ineffective patient stratification. What are needed are new immunotherapies that can overcome the limitations of existing therapeutic protocols.

### III. SUMMARY

Disclosed are methods related to treating cancers and metastasis with the administration of fucose and an agent that inhibits polio virus receptor.

In one aspect, disclosed herein are methods of treating inhibiting, decreasing, reducing, ameliorating, and/or preventing a cancer and/or metastasis (such as a breast cancer brain metastasis) in a subject comprising administering to the subject an agent that blocks polio virus receptor (such as, for example, an antibody (including, but not limited to a neutralizing antibody), antibody fragment (including, but not limited to, an sFv, Fv, Fab, Fab′, F(ab′)2, or other antigen-binding portion of an antibody), immunotoxin, small molecule, peptide, polypeptide, protein, siRNA, RNAi, and/or antisense oligonucleotide) and an agent that increases fucosylation (such as, for example, L-fucose, D-fucose, fucose-1-phosphate, and/or GDP-L-fucose).

Also disclosed herein are methods of detecting the presence of a metastasis or measuring the progression of a metastasis (including, but not limited to a brain metastasis) in a subject with cancer (such as, for example, breast cancer) comprising obtaining a tissue sample (such as, for example, a liquid biopsy wherein the biopsy comprises cerebral spinal fluid (CSF)) from a subject and measuring the amount of secreted fucosylated PVR in the sample (including, but not limited to measuring fucosylated PVR in cerebrospinal fluid), wherein the presence of or an increase in fucosylated PVR relative to a control indicates the presence of a brain metastasis.

In one aspect, disclosed herein are methods of treating, inhibiting, decreasing, reducing, ameliorating, and/or preventing a cancer and/or metastasis (such as a breast cancer brain metastasis) in a subject comprising obtaining a tissue sample (such as, for example, a liquid biopsy wherein the biopsy comprises cerebral spinal fluid (CSF)) from a subject; measuring the amount of secreted fucosylated PVR in the sample (including, but not limited to measuring fucosylated PVR in cerebrospinal fluid), wherein the presence of or an increase in fucosylated PVR relative to a control indicates the presence of a brain metastasis; and administering to the subject an agent that blocks polio virus receptor (such as, for example, an antibody (including, but not limited to a neutralizing antibody), antibody fragment (including, but not limited to, an sFv, Fv, Fab, Fab′, F(ab′)2, or other antigen-binding portion of an antibody), immunotoxin, small molecule, peptide, polypeptide, protein, siRNA, RNAi, and/or antisense oligonucleotide), and an agent that increases fucosylation (such as, for example, L-fucose, D-fucose, fucose-1-phosphate, and/or GDP-L-fucose).

Also disclosed herein are methods of treating, inhibiting, decreasing, reducing, ameliorating, and/or preventing a cancer and/or metastasis (such as a breast cancer brain metastasis) of any preceding aspect further comprising administering to the subject an anti-cancer agent (including, but not limited to immune checkpoint blockade inhibitor (including, but not limited to PD-1 inhibitors lambrolizumab, OPDIVO® (Nivolumab), BMS-936558, MDX1106, KEYTRUDA® (pembrolizumab), pidilizumab, cemiplimab, CT-011, and MK-3475; PD-L1 inhibitors TECENTRIQ® (Atezolizumab), BAVENCIO® (Avelumab), IMFINZI® (Durvalumab), MDX-1105 (BMS-936559), MPDL3280A, and MSB0010718C; and CTLA-4 inhibitors YERVOY® (ipilimumab) (MDX-010), and Tremelimumab (CP-675,206)).

In one aspect, disclosed herein are methods of treating, inhibiting, decreasing, reducing, ameliorating, and/or preventing a cancer and/or metastasis (such as a breast cancer brain metastasis) of any preceding aspect, further comprising administering to the subject an adoptive cell therapy (such as, for example, the transfer of tumor infiltrating lymphocytes (TILs), tumor infiltrating NK cells (TINKs), marrow infiltrating lymphocytes (MILs), chimeric antigen receptor (CAR) T cells, and/or CAR NK cells).

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, and 1C show that cancer associated fibroblasts exhibit high levels of fucosylation in breast tumors.

FIGS. 2A and 2B show the effect of depletion of fucosylated proteins on MDA-MB-231 cells in a wound healing assay and MTT assay. FIG. 2A shows how the media was depleted of fucosylated cells. FIG. 2B shows assay results. The assays show that brain metastasis-associated fibroblasts (bmCAFs) uniquely secrete fucosylated proteins that are required for stimulating breast cancer motility and proliferation.

FIG. 3 shows Genetic abrogation of fucosylation in bmCAFs by SLC35C1 knockout recapitulates conditioned media depletion experiments.

FIG. 4 shows global fucosylated proteomic profiling results identifying the polio virus receptor (PVR) as a significant bmCAF secreted, fucosylated protein.

FIG. 5 shows that loss of fucosylation impairs secretion of PVR from bmCAFs.

FIGS. 6A and 6B show by immunodepletion of PVR from conditioned media (6A) and knockdown of PVR in bmCAF (6B) that PVR drives breast cancer motility.

FIG. 7 shows that high PVR correlates with poor prognosis in triple negative breast cancer.

FIG. 8 shows that CAFs have higher fucosylation level as compared to BC cells.

FIG. 9 shows that BC-CAF secreted fucosylated proteins have tumorigenic properties.

FIG. 10 shows that PVR/CD155 is highly expressed and secreted in bmCAFs.

FIG. 11 shows that PVR is N-linked glycosylated-fucosylated.

FIG. 12 shows that serum PVR is high in cancer patients than normal controls.

FIG. 13 shows that secreted PVR potentially drives BC cells migration/invasion.

FIG. 14 shows that secreted PVR drives BC cells invasion.

FIG. 15 shows that PVR expression in bmCAF is required for enhanced tumor outgrowth in the brain.

FIGS. 16A, 16B, 16C, 16D, 16E, 16F, 16G, 16H, and 16I show BC CAFs exhibit high fucosylation levels that increase with tumor progression. FIG. 16A shows a representative microscopy image of a breast TMA histospot immunofluorescently (IF) stained for AAL (fucosylation marker), Pan-CK (BC marker), SMA+FAP (white, fibroblast marker cocktail), and DAPI (DNA/nuclei). Scale bars, 50 μm (top) and 100 μm (bottom). FIG. 16B shows scatterplots comparing total fucosylation levels in primary (left, blue) and patient-matched metastasis (right, red) TMA histospots (paired Student's t test). FIG. 16C shows box plot showing relative fucosylation levels in BC cells (green) and CAFs (blue) per TMA histospot (Student's t test). FIG. 16D shows scatterplots comparing fucosylation levels in tumor CAFs (tCAFs; left, blue) vs. patient-matched metastasis CAFs (mCAFs; right, red). Paired Student's t test. For (A)-(D), n=50 patients. FIG. 16E shows fucosylation signal within each SMA+FAP⁺ area plotted against the area of SMA+FAP⁺ regions per individual histospots (Spearman's r). FIG. 16F shows bright-field microscopy images of NBFs and patient-derived tCAFs and bmCAFs. Scale bar, 100 μm. FIG. 16G shows a Heatmap of NBF, tCAF, bmCAF, and normal astrocyte gene expression (RNA-seq data). FIG. 16H shows a Heatmap of fibroblast-, pericyte-, mesenchymal stem cell-, and fucosylation-associated gene expression (RNA-seq data) of indicated numbers of NBF, tCAF, and bmCAF lines. FIG. 16I shows Left: immunoblots of AAL-recognized fucosylation in three NBF, eight tCAF, and five bmCAF lines. Loading control: 0-tubulin. Right: AAL normalized to (3-tubulin levels.

FIGS. 17A, 17B, 17C, 17D, 17E, 17F, 17G, 17H, 17I, 17J, 17K, 17L, and 17M show PVR is a highly fucosylated protein secreted by bmCAFs. FIG. 17A shows motility as measured by percentage of wound closure of MDA-MB-231 and MDA-MB-468 cells treated with RPMI medium or fucosylated protein-depleted or replete CM from the indicated fibroblasts. Data shown as mean±SEM from three biological replicates (Student's t test). FIG. 17B shows a Heatmap shows fold change of top 23 proteins in fucosylated bmCAF vs. NBF secretomes. FIG. 17C shows an immunoblot (IB) of whole-cell lysate (WCL) and control vs. AAL lectin pull-down of NBF, tCAF, and bmCAF CM. FIG. 17D shows Left: IB showing elevated PVR levels at steady state in five bmCAF lines compared with three NBF and eight tCAF lines. Right: densitometry analysis of IB. FIG. 17E shows a dot plot showing PVR mRNA expression levels in stage I/II/III BC patient tumors from the brca_metabric dataset. Data shown as mean±SEM (Student's t test). FIG. 17F shows a box plot showing tumor expression levels of PVR mRNA in patients with single-organ metastasis vs. multi-organ metastases from the brca_mbcproject dataset (Student's t test). FIG. 17G shows a structural schematic of the four PVR isoforms. FIG. 17H shows (Left) Representative microscopy images of L-PLA staining for fucosylated PVR (red), phalloidin, and DAPI in coverslip-grown bmCAFs treated ±50 μM BrefA for 16 h. Scale bar, 100 m. (Right) Quantification of the number of fuco-PVR puncta per cell (n=400-600 nuclei). Data shown as mean±SEM (Student's t test). FIG. 17I shows equal amounts of bmCAF lysate treated ±PNGase F, followed by IB. FIG. 17J shows bmCAF CM subjected to control or AAL lectin bead pull-down ±500 mM L-fuc control wash, followed by IB for AAL (total fucosylated proteins, lower) or PVR (upper). FIG. 17K shows control vs. AAL pull-down of control (V2) or SLC35C1 knocked out (c1; fucosylation-deficient) bmCAF CM. WCL is derived from cell pellet. FIG. 17L shows IB of FLAG immunoprecipitates from WCL or CM from DDKEV (FLAG-empty vector [EV])-expressing or DDKsPVRγ (FLAG-tagged secreted PVRγ)-expressing bmCAFs. FIG. 17M shows L-PLA confirms direct fucosylation of PVR in DDKsPVRγ-expressing bmCAFs using AAL and anti-FLAG antibody. Scale bar, 100 μm. FIGS. 17C, 17H, 17I, 17J, 17K, and 17M are either a representative blot or microscopy image of three biological replicates performed.

FIGS. 18A, 18B, 18C, 18D, 18E, 18F, 18G, 18H, 18I, and 18J, show that PVR fucosylation is induced by FUT11, a key hypoxia- and HIF1α-regulated gene. FIG. 18A shows Left: heatmap showing the mRNA expression levels of 13 FUTs across five different bmCAF lines (RNA-seq data). Right: RT-qPCR analysis of the mRNA expression levels of FUT1 (negative control), FUT4, FUT8, FUT10, and FUT11 in bmCAF. FIG. 18B shows dot plots showing the mRNA expression levels of FUT1, FUT4, FUT8, FUT10, and FUT1I in stage I/II/III BC patients (brca_metabric dataset) (Student's t test). FIG. 18C shows predicted hypoxia response element (HRE) with HIF1α-binding motif (red) in the FUT11 5′ promoter. FIG. 18D shows IB showing induction of HIF1α in bmCAFs only during hypoxia at 24 h and 48 h. Asterisk indicates a non-specific band. FIG. 18E shows a time course of mRNA expression of FUT11 in response to hypoxia as measured by RT-qPCR (Student's t test). FIG. 18F shows ChIP-RT-qPCR assay using anti-HIF1α antibody to verify HIF1α binding to the HRE element within the FUT11 promoter during hypoxia (48 h) (Student's t test). FIG. 18G shows IB of WCL and control vs. AAL lectin pull-down of NBF, tCAF, and bmCAF CM under hypoxia (48 h) vs. normoxia (numbers in red denote densitometry values of PVR bands). FIG. 18H shows the number of fuco-PVR puncta measured per cell (left) by L-PLA of coverslip-grown shNT-expressing vs. shFUTl1-expressing bmCAFs (right). Data shown as mean±SEM derived from four different fields in one representative experiment of three biologically independent experiments (n=200-400 nuclei; Student's t test). FIG. 18I shows IB of WCL and control vs. AAL LPD showing induction of sfPVR under hypoxia (48 h) vs. normoxia in shNT-expressing but not in shFUT11-expressing bmCAF CM. (numbers in red denote densitometry values of PVR bands). FIG. 18J shows gene set enrichment analysis (GSEA) of the Breast Invasive Carcinoma dataset (TCGA, Pan Cancer Atlas) correlations of FUT11 or PVR expression with hypoxia, secretion, and EMT signatures. Data shown in 18A, 18E, and 18F represent mean±SEM derived from three biological replicates. Shown in 18D, 18G, and 18I is a representative blot of three biological replicates performed.

FIGS. 19A, 19B, 19C, 19D, 19E, and 19F show phosphoproteomic analysis of BC cells reveals that sfPVR drives migration/invasion signaling in BC cells. FIG. 19A shows a schematic of phosphoproteomic analysis of MDA-MB-231 cells treated with RPMI or control (ctrl) or PVR-immunodepleted CM. Three biological replicates of BC cells for each treatment were generated for analysis by LC-MS/MS. FIG. 19B shows principal component analysis of phosphopeptides of the BC cells treated in (19A). FIG. 19C shows (Upper) Volcano plot showing the phosphoproteome changes of MDA-MB-231 treated with ctrl bmCAF CM vs. RPMI. (Lower) Volcano plots showing phosphopeptides in MDA-MB-231 cells that were downregulated (left) or upregulated (right) by ctrl bmCAF CM compared to RPML. Blue or red indicates ctrl bmCAF CM-altered phosphopeptides that were reverted in abundance by PVR-depleted bmCAF CM. FIG. 19D shows pathway analysis using KEGG or gene ontology of phosphoproteins that were altered by ctrl bmCAF CM and reverted by PVR-depleted CM. FIG. 19E shows a PPI network of phosphoproteins significantly altered by bmCAF sfPVR generated by STRING and visualized by Cytoscape. FIG. 19F shows GSEA of the Breast Invasive Carcinoma dataset (TCGA, Pan Cancer Atlas) showing that key phosphoproteins identified in our data (TJP1, EPHA2, PTPN12, AHNAK, ARHGEF2) correlate with EMT and apical junction signatures.

FIGS. 20A, 20B, 20C, 20D, 20E, 20F, and 20G show that sfPVR-stimulated BC invasion is associated with altered focal adhesion, actin cytoskeleton, and tight junction dynamics. FIG. 20a shows representative microscopy images of BC cells in wound-healing assay at 16 h. Scale bar, 50 μm. FIG. 20B shows quantification of percent wound closure at 16 h from (A) (Student's t test). FIG. 20C shows representative microscopy images of BC cells invaded through Matrigel-coated transwell membranes at 16 h. RPMI or indicated bmCAF CM were used as chemoattractants. Scale bar, 50 μm. FIG. 20D shows quantification of invaded BC cells from (C) (Student's t test). FIG. 20E shows (Upper) Representative microscopy images: BC cells treated with RPMI or indicated bmCAF CM were IF stained for pFAK. Scale bar, 100 μm. (Lower) Quantification of the number of pFAK puncta per cell. MDA-MB-231, n=120-160 nuclei; MDA-MB-231bm, n=1,000-1,500 nuclei. Data shown are mean±SEM of six fields (Student's t test). FIG. 20F shows (Upper) Representative microscopy images of BC cells treated with RPMI or indicated bmCAF CM showing TJP1- and CLD1-containing complexes as PLA puncta. Scale bar, 100 μm. (Lower) Quantification of the number of PLA puncta per cell. n=900-1800 nuclei. Data shown are mean±SEM of ten fields (Student's t test). FIG. 20G shows (Upper) Representative microscopy images of phalloidin staining of BC cells treated with RPMI or indicated bmCAF CM showing cytoskeletal structure/spread and actin stress fibers. Scale bar, 150 μm. (Lower) Quantification of cell-spread area as measured by Fiji software. n=50 cells (Student's t test). In (20B)-(20G), data are shown as mean±SEM derived from three independent biological replicate experiments.

FIGS. 21A, 21B, 21C, 21D, and 21E show that sfPVR promotes metastatic spread of BC cells in the brain. FIG. 21A shows a schematic of experimental design for our intracranial BCBM model. Six-week-old NSG mice were implanted with 104 luciferase-expressing MDA-MB-231bm cells or mixed with bmCAFs at 1:1 ratio (104 BC+104 bmCAF cells) by stereotactic surgery. FIG. 21B shows representative in vivo bioluminescence images of mouse brain at day 28 (endpoint) using D-luciferin substrate and the IVIS imaging system. FIG. 21C shows bioluminescence kinetics (mean luminescence signal ±SEM) at the model endpoint (day 28) of indicated tumor/CAF combinations (and mouse numbers) shown in the dot plots as photons per second (p/s) per region of interest (ROI) (Student's t test). FIG. 21D shows representative microscopy images of H & E (upper) or IF (middle and lower) stained BCBM tumors and micrometastases (BC marker: Pan-CK; fibroblast marker: SMA; nuclei: DAPI). Scale bars, 50 μm (H & E), 100 μm (injection site), and 150 μm (micrometastases). FIG. 21E shows dot plots show quantification of distance (left) and number (right) of micrometastases outside of the injection sites. Data shown are mean±SEM derived from the brain sections of three individual mice per treatment group (Student's t test).

FIGS. 22A, 22B, 22C, 22D, 22E, 22F, 22G, 22H, and 22I show that Fuco-PVR is high in bmCAFs in BCBM patient tissue. FIG. 22A shows (Upper) Microscopy images of one representative BCBM core biopsy (n=10 patients) multiplex IF stained for the indicated markers (fibroblast: SMA+FAP [white]; BC cells: Pan-CK; total PVR: anti-PVR; nuclei: DAPI). Scale bar, 50 μm. (Lower) Magnified crop. Yellow arrows indicate bmCAFs. Scale bar, 150 μm. FIGS. 22B and 22C show (22B) stacked bar charts and (22C) scatterplots showing average total PVR signal (arbitrary fluorescence units) per cell in BC cells (red) vs. bmCAFs (blue) in each tumor across the ten patients (paired Student's t test). FIG. 22D shows (Upper) Representative microscopy images of BCBM tissue sections multiplex IF stained for the indicated markers (fibroblast: SMA+FAP [white]; BC cells: Pan-CK; fuco-PVR: L-PLA-PVR; nuclei: DAPI). (Lower) Magnified crop. Yellow arrows indicate bmCAFs. Scale bar, 150 μm. FIGS. 22E and 22F show (22E) Stacked bar charts and (22F) scatterplots showing average total fuco-PVR signal (arbitrary fluorescence units) per cell in BC cells (red) vs. bmCAFs (blue) in each tumor across the ten patients (paired Student's t test). FIG. 22G shows bar charts showing percentage of fuco-PVR-bmCAFs in the tumors of the ten BCBM patients. FIG. 22H shows bar charts showing percentage of fuco-PVR signal over total PVR signal in bmCAFs in the tumors of the ten BCBM patients. FIG. 22I shows the segregated percentage levels of bmCAFs (left) and fuco-PVR (right) within bmCAFs in the brain tumors of young (n=6) vs. old (n=4) BCBM patients (Student's t test).

FIGS. 23A, 23B, 23C, 23D, and 23E show characterization of fucosylation status of BC CAFs. FIG. 23A shows representative microscopy images of MDA-MB-231 xenograft sections IF stained to characterize different subtypes of fucosylation (fucosylation AAL: α(1,2), α(1,3), α(1,4) & α(1,6), UEA-1: α(1,2), and LTL: α(1,3) & α(1,4)), BC cells: Pan-CK, fibroblasts: SMA (white)). Scale bar, 50 μM. FIG. 23B shows a representative microscopy image of MDA-MB-231 BC tumor IF stained with AAL (left). Scale bar, 50 μM. (right) Relative core fucosylation as measured by AAL in pan-CK+BC vs. SMA+CAFs. Data are shown as mean±SEM derived from 3 individual tumors (Student's t-test).

FIG. 23C shows a representative microscopy image of MDA-MB-231 BC tumor sections IF stained with AAL±500 mM L-fucose (L-fuc) control wash. Loss of AAL signal in L-fuc-washed tissue confirms specificity of AAL for L-fuc. Scale bar, 100 μM. FIG. 23D shows representative microscopy images of breast tumor tissue microarray (TMA; n=50 patient-matched primary and metastasis histospot pairs) histospots with high vs. low CAF populations IF stained for AAL (fucosylation), pan-cytokeratin (breast cancer marker; Pan-CK), SMA+FAP cocktail (white, fibroblast marker) and DAPI (DNA/nuclei). Scale bars, 50 μM (left) and 100 μM (right). FIG. 23E shows (left) Boxplots showing increased fibroblast populations with increased primary tumor staging. Scatter plots comparing (center) total CAF populations or (right) % of CAFs in primary (blue circles, n=50) vs. patient-matched metastatic (red circles, n=50) tumor samples (Student's t-test).

FIGS. 24A, 24B, and 24C show characterization of fucosylation status of BC CAFs. FIG. 24A shows principal component analysis (PCA) of gene expression of NBFs, tCAFs, bmCAFs, and astrocytes. Gene expression profile of each cell type clustered together and are significantly different from each other, suggesting their uniqueness. FIG. 24B shows pathway analysis using MSigD or KEGG of 258 significantly differentially expressed genes in bmCAFs compared with tCAFs. FIG. 24C shows IB showing global fucosylation levels detected by AAL (α(1,2), α(1,3), α(1,4) & α(1,6)) (left), UEA-1 (α(1,2)) (middle), or LTL (α(1,3) & α(1,4)) (right) lectins in NBFs, tCAFs, and bmCAFs cells. b-tubulin is the loading control.

FIGS. 25A, 25B, 25C, 25D, and 25E show bmCAF-secreted, -fucosylated proteins promote BC cells migration. FIG. 25A shows a schematic for depletion of fucosylated proteins from fibroblast conditioned media (CM). The CM from the indicated fibroblasts were depleted of fucosylated proteins by incubation overnight with AAL (fucosylation-binding; fucosylated protein-depleted CM) or control (ctrl; fucosylation protein-replete CM) beads. FIG. 25B shows IB of fucosylated proteins in NBF, tCAFs, and bmCAF secretomes as detected by AAL (left) and UEA-1 (right) pulldown. FIG. 25C shows cell viability of MDA-MB-231 and MDA-MB-468 cells treated with RPMI media or fucosylated protein-depleted or replete CM from the indicated fibroblasts was measured by XTT (72 h) (Student's t-test). FIG. 25D shows representative microscopy images of MDA-MB-231 and MDA-MB-468 treated with RPMI media or fucosylated protein-depleted or replete CM from the indicated fibroblasts as assessed at 0 h and 16 h after initiation of wound healing assay. Scale bar, 50 μM. FIG. 25E shows cell viability of MDA-MB-231 and MDA-MB-468 cells treated with RPMI media or fucosylated protein-depleted or replete CM from the indicated fibroblasts was measured by XTT (16 h). Data is shown as mean±SEM derived from 3 independent biological replicate experiments.

FIGS. 26A, 26B, 26C, and 26D show bmCAF-secreted, -fucosylated proteins promote BC cell migration. FIG. 26A shows representative microscopy images of MDA-MB-231 and MDA-MB-468 cells treated with RPMI media or CM from V2 or Cl bmCAFs at 16 h in a wound healing assay. FIG. 26B shows the motility of MDA-MB-231 and MDA-MB-468 cells treated with RPMI or CM from V2 or Cl bmCAFs. Data are shown as mean±SEM derived from 3 independent biological replicate experiments (Student's t-test). FIG. 26C shows IB confirming ~70% loss of core fucosylation (AAL) in SLC35C1 knockout (C1; fucosylation-deficient) vs. control (V2) bmCAFs. FIG. 26D shows schematic of Fucose Salvage pathway. L-fucose up-taken into cells is phosphorylated by FUK and GDP-coupled by FPGT, yielding the GDP-fucose substrate, which is transported into the ER/Golgi via SLC35C1/2 transporters, where it is conjugated onto proteoglycans by 13 fucosyltransferases. Yellow circle highlights the transporter that was knocked out using CRISPR to abrogate protein fucosylation in bmCAFs.

FIGS. 27A, 27B, 27C, 27D, 27E, 27F, 27G, 27H, and 27I show that PVR is a highly fucosylated protein that is secreted by bmCAFs. FIG. 27A shows a schematic of NBF vs. bmCAF secretomes subjected to control vs. AAL pulldown and LC/MS-MS. Venn diagram shows the number of AAL-pulled down proteins that are unique or overlapping in NBF (blue) and bmCAF (yellow) secretomes. Pulldown and LC/MS-MS was performed on 3 independent biological replicates; numbers represent consistent hits across all 3 replicates. FIG. 27B shows a KM plot showing poorer survival probability of BC patients with high tumor expression levels of PVR (red) vs. low PVR levels (black). FIG. 27A shows an exon schematic of 2 transmembrane (tPVR; PVRa and PVRd) and 2 secreted (sPVR; PVRb and PVRg) PVR isoforms. FIG. 27D shows a schematic of lectin-mediated proximity ligation assay (L-PLA) used for IF staining of fuco-PVR. FIG. 27E shows representative microscopy images of fuco-PVR visualized by L-PLA in coverslip-grown NBF, tCAF, and bmCAF cells (left). Scale bar, 50 μM. Number of fuco-PVR per cell (cell number was counted by nuclei; right), n=500-1000 nuclei (Student's t-test). FIG. 27F shows representative microscopy images of coverslip-grown bmCAFs subjected to AAL staining±500 mM L-fuc wash. Scale bar, 50 μM. FIG. 27G shows predicted N-glycosylation sites of PVR and their threshold scores (NetNGlyc predictive software). Numbers highlighted in red (9/9) and with “*” represent sites have high probability to be N-glycosylated. FIG. 27H shows a DNA gel image showing PCR-based cloning of PVR bmCAF-derived cDNA. FIG. 27I shows representative microscopy images of fuco-PVR visualized by L-PLA using AAL and anti-PVR antibody in DDKsPVRg-expressing bmCAFs treated for 16 h with 50 μM Brefeldin A vs. DMSO treatment. Scale bar, 100 μM. n=500-1000 nuclei. Data are shown as mean±SEM derived from 3 independent biological replicate experiments (Student's t-test).

FIGS. 28A, 28B, 28C, and 28D show PVR is a highly fucosylated protein that is secreted by bmCAFs. FIG. 28A shows IB of WCL (left), FLAG pulldown (middle), and control vs. AAL lectin-pulldown (right) of DDKsPVRg-expressing bmCAFs CM under ±250 μM 2FF (72 h), showing that 2FF reduces AAL by ~2-fold in DDKsPVRg-expressing bmCAFs (densitometry of the AAL bands is shown in red as measured by ImageJ), and complete loss of PVR secretion is observed in 2FF-treated samples. FIG. 28B shows (upper) Pathway analysis using Gene Ontology of 24 putative DDKsPVRg-interacting proteins that were affected by the fucosylation in bmCAFs. (lower) Individual proteins in the protein stabilization and chaperone pathway identified in the screen. FIG. 28C shows a protein-protein interaction network of 3 identified hits generated by STRING and visualized by Cytoscape. Red-colored proteins (HSP90B1 and HSP1B) are the hits from our data and all other proteins are STRING-predicted interactors. Distinct clusters ER chaperone, exosome, and ER-Golgi intermediate compartment proteins are shown. FIG. 28D shows IB of HSP90B1 (top) and HSPA1B (bottom) of FLAG-immunoprecipitates (left) from DDKsPVRg-expressing bmCAFs treated ±250 μM 2FF (72 h). (right) WCL input and ponceau as a loading control (densitometry of HSP90B1 bands is shown in red as measure by imageJ). Shown is a representative IB image out of 3 independent biological replicates.

FIGS. 29A, 29B, 29C, 29D, 29E, 29F, and 29G show that PVR fucosylation is catalyzed by FUT11, a key hypoxia- and HIF1α-regulated gene. FIG. 29A shows qRT-PCR analysis of FUT1, 4, 8, 10, and 11 expression levels in NBFs (left) and tCAFs (right). Data are shown as mean±SEM derived from 3 independent biological replicates. FIG. 29B shows KM plot showing poorer survival probability of BC patients with high tumor expression levels of FUT11 (red) vs. low FUT11 levels (black). FIG. 29C shows a comparison of FUT11 mRNA expression levels in patients exhibiting single-organ (green) vs. multi-organ (blue) metastases using a publicly available dataset (brca_mbcproject). Data shown as mean±SEM (Student's t-test). D. qRT-PCR analysis of FUT1, 4, 8, 10, and 11 expression levels in NBFs, tCAFs, and bmCAFs in response to hypoxia (48 h). Data are shown as mean±SEM derived from 3 independent biological replicate experiments (Student's t-test). FIG. 29E shows representative IF stained microscopy images showing HiF1a induction under 0.5% 02 hypoxia for 48 h in coverslip-grown NBFs and tCAFs (left). Scale bar, 100 μM. Quantification of nuclear HIF1α (right). n=40-80 nuclei. Data are shown as mean±SEM derived from 3 independent biological replicate experiments (Student's t-test). FIG. 29F shows qRT-PCR analysis confirming knockdown of FUT11 in bmCAFs. FIG. 29G shows a pearson correlation analysis of PVR and FUT11 expression from brca_mbcproject_2022 dataset. Higher r values in patients with brain met or multi-organ mets reflect a stronger correlation between the mRNA expression levels of PVR and FUT11.

FIGS. 30A, 30B, 30C, 30D, 30E, 30F, 30G, and 30H show that sfPVR drives BC cell migration/invasion by modulating focal adhesion, actin cytoskeleton and tight junction dynamics. FIG. 30A shows IB confirming immunodepletion of PVR from bmCAF CM that was used for the phosphoproteomic experiments shown in FIG. 19. FIG. 30B shows IB confirming knockdown of PVR using shPVR in bmCAFs. Quantification of PVR relative to b-tubulin (right) shows a 70% knockdown of PVR by shPVR2. FIG. 30C shows (left) Representative microscopy images of colony formation assay of MDA-MB-231 or MDA-MB-231bm cells treated with CM from shNT or shPVR bmCAFs. (Right) Quantification of cell density. Data are shown as mean values ±SEM derived from 3 independent biological replicate experiments (Student's t-test). FIG. 30D shows a schematic for the matrigel invasion assay used in FIG. 20C in which CM from bmCAFs was used as a chemoattractant. FIG. 30E shows (Left) Representative microscopy images of coverslip-grown MDA-MB-231 or MDA-MB-231bm cells treated with CM from control (DDKEV) or sPVRg-overexpressing (DDKsPVRg) bmCAFs that were IF stained for pFAK, phalloidin, and DAPI. Scale bar, 100 μM. (Right) Quantification of pFAK punctae per cell as measured by Fiji software. MDA-MB-231, n=770-780 nuclei; MDA-MB-231bm, n=1400-1900 nuclei (Student's t-test). FIG. 30F shows (Left) Representative microscopy images of MDA-MB-468 cells treated with RPMI or CM from ctrl (shNT & DDKEV), PVR knockdown (shPVR), or sPVRg-overexpressing (DDKsPVRg) bmCAFs that were IF stained for pFAK, phalloidin, and DAPI. Scale bar, 150 μM. (Right) Quantification of pFAK punctae per cell as measured by Fiji software. n=250-850 nuclei (Student's t-test). FIG. 30G shows (Left) Representative microscopy images of MDA-MB-231 and MDA-MB-231bm cells treated with CM from DDKEV- or DDKsPVRg-bmCAFs and PLA stained for TJP1- and CLD1-containing complexes. Scale bar, 150 μM. (Right) Quantification of PLA punctae per cell as measure by Fiji software. n=1,000-2,000 nuclei (Student's t-test). FIG. 30H shows (Left) Representative microscopy images of MDA-MB-468 cells treated with CM from shNT-, DDKEV-, shPVR-, or DDKsPVRg-expressing bmCAFs and PLA stained for TJP1- and CLD1-containing complexes. Scale bar, 150 μM. (Right) Quantification of PLA puntae per cell as measure by Fiji software. n=250-450 nuclei (Student's t-test). For all panes 30E-30H, all data are shown as mean±SEM derived from 3 independent biological replicate experiments.

FIGS. 31A, 31B, 31C, 31D, and 31E show that sfPVR drives BC cells migration/invasion by modulating focal adhesion, actin cytoskeleton and tight junction dynamics. FIG. 31A shows a dot plot showing TJP1 expression levels in stage I, II, and III BC patients, analyzed from the brca_metabric dataset. Data are shown as mean±SEM (Student's t-test). FIG. 31B shows the Pearson correlation analysis of PVR and TJP1 expression from brca_metabric dataset. Negative r value reflects a negative correlation between the mRNA expression levels of PVR and TJP1 in tumors of BC patients. FIG. 31C shows an example microscopy image illustrating the measurement of cellular spread area using Fiji software. FIG. 31D shows additional representative microscopy images of coverslip-grown MDA-MB-231 and MDA-MB-231bm cells treated with RPMI or CM from shNT- or shPVR-expressing bmCAFs and IF stained for phalloidin (white), showing the cellular spread area and actin stress fibers. Scale bar, 100 μM. FIG. 31E shows (Upper) Representative microscopy images of coverslip-grown MDA-MB-231 and MDA-MB-231bm cells treated with RPMI or CM from shNT- or shPVR-expressing bmCAFs in a FITC-gelatin degradation assay (BC cells were stained by phalloidin, red). Scale bar, 100 μm. (Bottom) Quantification of FITC gelatin (green) areas degraded by MDA-MB-231 and MDA-Mb-231bm. Data shown as mean % area degraded (loss of green signal, ±SEM) per field (n=5 fields) from 3 independent biological replicate experiments (Student's t-test).

FIGS. 32A, 32B, 32C, 32D, and 32E show that sfPVR drives BC cells migration/invasion. FIG. 32A shows a schematic of DTSSP crosslinking and LC-MS/MS or immunoblotting of MDA-MB-231 cells treated with CM from ctrl (DDKEV) or sPVRg-overexpressing (DDKsPVRg) bmCAFs. FIG. 32B shows FLAG IP and IB of PVR from MDA-MB-231 cell lysates confirming successful IP of the crosslinked DDKsPVRg. *n.s.: non-specific band (32C) List of proteins that were identified by LC-MS/MS as significantly bound by DDKsPVRg vs. DDKEV. FIG. 32D shows a protein-protein interaction network of 2 identified hits generated by STRING and visualized by Cytoscape. Red-colored proteins (MTMR2 and MYO1G) are the hits from our data and all other proteins are STRING-predicted interactors. Distinct clusters of endosomal membrane associated, phagocytic vesicle membrane associated, and prosynaptic actin cytoskeleton associated proteins are shown. FIG. 32E shows IB of MYO1G (top) and MTMR2 (bottom) of WCL Input (left) and FLAG-immunoprecipitates (right) of MDA-MB-231 cells treated with CM from control (DDKEV) or DDKsPVRg-overexpressing bmCAFs (densitometry of FLAG IP MTMR2 bands is shown in red as measure by imageJ). Shown is a representative IB image out of 3 independent biological replicates.

FIGS. 33A, 33B, and 33C show that sfPVR drives BC cell migration/invasion in the brain. FIG. 33A shows (Upper) Illustration of location of the injection site on the mouse brain surface (green circle) and the sectioning (dashed black lines) for histology analysis. (Bottom) Example image illustrating the measurement of distal spread and count of micrometastases in the brain parenchyma using Fiji software. FIG. 33B shows additional representative microscopy images of BCBM tumors subjected to H & E and IF staining for Pan-CK (BC cells), SMA (fibroblasts), and DAPI (nuclei). Injection sites are indicated by dashed yellow lines; micrometastases are indicated by yellow arrows. FIG. 33C shows representative microscopy images of brain sections subjected to H & E staining showing increased micrometastases in brains of mice injected with MDA-MB-231bm+shNT bmCAFs and MDA-MB-231bm+DDKsPVRg bmCAFs compared to controls.

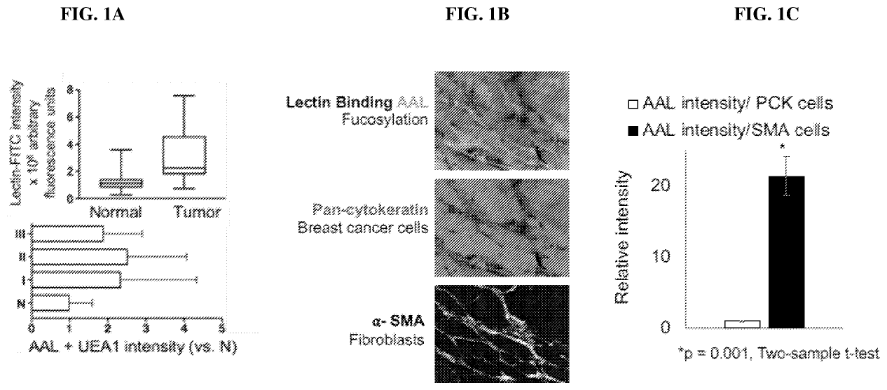

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A number of embodiments of the invention have been described. Nevertheless, it will be understood that various modifications may be made without departing from the spirit and scope of the invention. Accordingly, other embodiments are within the scope of the following claims.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims that follow, reference will be made to a number of terms which shall be defined to have the following meanings:

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “biological sample” or “tissue sample” refers to any portion of biological material from a subject to be used in any of the methods or as a part of any of the compositions disclosed herein including, but not limited to, tissue biopsy, whole blood, serum, plasma, peripheral blood mononuclear cells, urine sample, lung lavage, sputum, saliva, cerebrospinal fluid, and fecal sample. The biological can include samples for normal and cancerous tissue. Sample may be obtained from any tissue a subject by any means known in the art (tissue resection, biopsy phlebotomy, core biopsy).

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, fucoidan, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

A “binding molecule” or “antigen binding molecule” (e.g., an antibody or antigen-binding fragment thereof including, but not limited to an sFv, Fv, Fab, Fab′, F(ab′)2, or other antigen-binding portion of an antibody)) as provided herein refers in its broadest sense to a molecule that specifically binds an antigenic determinant. In one embodiment, the binding molecule specifically binds to an immunoregulator molecule (such as for example, a transmembrane SEMA4D (CD100) polypeptide of about 150 kDa or a soluble SEMA4D polypeptide of about 120 kDa). In another embodiment, a binding molecule is an antibody or an antigen binding fragment thereof, e.g., MAb 67 or pepinemab.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods and Compositions

Fucosylation, the post-translational modification of proteins with the dietary sugar L-fucose, is a mechanism that is well established for its importance in immune cell biology and organ developmental processes but that is poorly understood in terms of its roles in cancer. Fucose is transported extracellularly through the plasma membrane, where it is first phosphorylated by fucokinase (FUK). Then it is conjugated with GDP, yielding GDP-Fucose, which is a usable form in the cell. GDP-Fucose is transported into the ER/Golgi through SLC35C1/2, where it can be conjugated to a serine/threonine via an oxygen, which is referred to as O′-linked fucosylation, or to an arginine via a nitrogen, which is referred to as N′-linked fucosylation. The fucosylated protein can then be either trafficked to the cytoplasm or the cell surface. Global fucosylation is reduced during progression in human melanomas (UEA1 fucose-binding lectin staining analysis of tumor microarray (TMA; n=~300 patients)) via an ATF2-mediated transcriptional repression of fucokinase (FUK). Importantly, increasing fucosylation by genetic manipulation of tumor cells or by dietary L-fucose supplementation significantly blocks tumor growth and metastasis by >50% in mouse models. The studies herein demonstrate that i) tumor fucosylation levels can be used to identify different stages of cancer, and ii), the manipulation of fucosylation represents a feasible anti-cancer approach.

As shown herein, fucosylation of polio virus receptor (PVR) is a good marker for the presence of a metastasis (such as a breast cancer brain metastasis). This, in one aspect, disclosed herein are methods of detecting the presence of a brain metastasis or measuring the progression of a brain metastasis in a subject with breast cancer comprising obtaining a tissue sample from a subject and measuring the amount of secreted fucosylated PVR in the sample, wherein the presence of or an increase in fucosylated PVR relative to a control indicates the presence of a brain metastasis. Accordingly, in one aspect, disclosed herein are methods of detecting the presence of a metastasis or measuring the progression of a metastasis (including, but not limited to a brain metastasis) in a subject with cancer (such as, for example, breast cancer) comprising obtaining a tissue sample (such as, for example, a liquid biopsy wherein the biopsy comprises cerebral spinal fluid (CSF)) from a subject; measuring the amount of secreted fucosylated PVR in the sample (including, but not limited to measuring fucosylated PVR in cerebrospinal fluid), wherein the presence of or an increase in fucosylated PVR relative to a control indicates the presence of a brain metastasis

It is understood and herein contemplated that the detection of a metastasis (including, but not limited to a brain metastasis such as, for example breast cancer brain metastasis) highlights the need for treatment of the metastasis. Also, as shown herein reducing or decreasing fucosylation alone or in combination with other treatment strategies can be useful in the treatment of a cancer and/or metastasis (including, but not limited to breast cancer brain metastasis). Accordingly, in one aspect, disclosed herein are methods of treating, inhibiting, decreasing, reducing, ameliorating, and/or preventing a cancer and/or metastasis (such as a breast cancer brain metastasis) in a subject comprising obtaining a tissue sample (such as, for example, a liquid biopsy wherein the biopsy comprises cerebral spinal fluid (CSF)) from a subject; measuring the amount of secreted fucosylated PVR in the sample (including, but not limited to measuring fucosylated PVR in cerebrospinal fluid), wherein the presence of or an increase in fucosylated PVR relative to a control indicates the presence of a brain metastasis; and administering to the subject an agent that blocks polio virus receptor (such as, for example, an antibody (including, but not limited to a neutralizing antibody), antibody fragment (including, but not limited to, an sFv, Fv, Fab, Fab′, F(ab′)2, or other antigen-binding portion of an antibody), immunotoxin, small molecule, peptide, polypeptide, protein, siRNA, RNAi, and/or antisense oligonucleotide), and an agent that increases fucosylation (such as, for example, L-fucose, D-fucose, fucose-1-phosphate, and/or GDP-L-fucose).

The disclosed methods for treatment are not necessarily tied to the detection method and it is understood can be used to treat any cancer or metastasis once diagnosed. Thus, in one aspect, disclosed herein are methods of treating a treating, inhibiting, decreasing, reducing, ameliorating, and/or preventing a cancer and/or metastasis (such as a breast cancer brain metastasis) in a subject comprising administering to the subject an agent that blocks polio virus receptor (such as, for example, an antibody (including, but not limited to a neutralizing antibody), antibody fragment (including, but not limited to an sFv, Fv, Fab, Fab′, F(ab′)2, or other antigen-binding portion of an antibody)), immunotoxin, small molecule, peptide, polypeptide, protein, siRNA, RNAi, and/or antisense oligonucleotide) and an agent that increases fucosylation (such as, for example, L-fucose, D-fucose, fucose-1-phosphate, and/or GDP-L-fucose).

The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) used in the disclosed methods can be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the fucose comprising compositions into the nose and nasal passages through one or both of the nares and can comprise delivery by a spraying mechanism or droplet mechanism, or through aerosolization of the nucleic acid or vector. Administration of the fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) by inhalant can be through the nose or mouth via delivery by a spraying or droplet mechanism. Delivery can also be directly to any area of the respiratory system (e.g., lungs) via intubation. The exact amount of the fucose comprising compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions), if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The fucose comprising compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions for oral administration include powders or granules, suspensions or solutions in water or non-aqueous media, capsules, sachets, or tablets. Thickeners, flavorings, diluents, emulsifiers, dispersing aids or binders may be desirable.

Some of the fucose modulating compositions (including, but not limited to fucose (such as, for example L-fucose, D-fucose, fucose-1-phosphate, or GDP-L-fucose) and fucose comprising compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the fucose comprising compositions may be determined empirically, and making such determinations is within the skill in the art. The dosage ranges for the administration of the fucose comprising compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

The disclosed methods of treating, inhibiting, reducing, decreasing, ameliorating, and/or preventing cancer and/or metastasis can be used to treat any disease where uncontrolled cellular proliferation occurs such as cancers. Thus, in one aspect disclosed herein are methods of treating, inhibiting, decreasing, reducing, ameliorating, and/or preventing a cancer and/or metastasis (such as, for example, a melanoma) in a subject comprising administering to the subject an agent that an agent that modulates (including increases) the amount of fucosylation on the cell (such as a fucose including, but not limited to L-fucose, D-fucose, fucoidan, fucose-1-phosphate, GDP-L-fucose, or L-fucose/GDP-L-fucose analogues). A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer. The methods disclosed herein may also be used for the treatment of precancer conditions such as cervical and anal dysplasias, other dysplasias, severe dysplasias, hyperplasias, atypical hyperplasias, and neoplasias. As noted herein, the disclosed methods are particularly useful in cancers metastatic breast cancer (including, but not limited to breast cancer brain metastasis).

In one aspect, it is understood and herein contemplated that administration of an agent that increases fucosylation (such as, for example, L-fucose, D-fucose, fucose-1-phosphate, or GDP-L-fucose) and PVR inhibitor alone may not be sufficient to control a cancer. Thus, disclosed herein are methods of treating a treating, inhibiting, decreasing, reducing, ameliorating, and/or preventing a cancer and/or metastasis (such as, for example, melanoma) in a subject comprising administering to the subject fucose (such as for example, L-fucose, D-fucose, fucoidan, fucose-1-phosphate, GDP-L-fucose, or L-fucose/GDP-L-fucose analogues) and an inhibitor of PVR, further comprising the administration of an anti-cancer agent or immune checkpoint inhibitor (such as, for example, PD1/PDL1 blockade inhibitors and/or CTLA4/B7-1 or 2 inhibitors (including, but not limited to the PD-1 inhibitors lambrolizumab, OPDIVO® (Nivolumab), BMS-936558, MDX1106, KEYTRUDA® (pembrolizumab), pidilizumab, cemiplimab, CT-011, and MK-3475; PD-L1 inhibitors TECENTRIQ® (Atezolizumab). BAVENCIO® (Avelumab), IMFINZI® (Durvalumab), MDX-1105 (BMS-936559), MPDL3280A, and MSB0010718C; and CTLA-4 inhibitors YERVOY® (ipilimumab) (MDX-010), and Tremelimumab (CP-675,206) or any other anti-cancer agent disclosed herein), adoptive cell therapies, and/or CAR T therapies.

As noted above, the disclosed methods of treating a cancer with an agent that an agent that modulates (including increases) the amount of fucosylation on the cell (such as a fucose including, but not limited to L-fucose, D-fucose, fucoidan, fucose-1-phosphate, GDP-L-fucose, or L-fucose/GDP-L-fucose analogues) and a PVR inhibitor contemplate the co-administration of an anti-cancer agent. The anti-cancer agent can comprise any anti-cancer agent known in the art including, but not limited to antibodies, tumor infiltrating lymphocytes, checkpoint inhibitors, dendritic cell vaccines, anti-cancer vaccines, immunotherapy, and chemotherapeutic agents. In one aspect, the anti-cancer agent can include, but is not limited to Abemaciclib, Abiraterone Acetate, ABITREXATE® (Methotrexate), ABRAXANE® (Paclitaxel Albumin-stabilized Nanoparticle Formulation), ABVD, ABVE, ABVE-PC, AC, AC-T, ADCETRIS® (Brentuximab Vedotin), ADE, Ado-Trastuzumab Emtansine, ADRIAMYCIN® (Doxorubicin Hydrochloride), Afatinib Dimaleate, AFINITOR® (Everolimus), AKYNZEO® (Netupitant and Palonosetron Hydrochloride), ALDARA® (Imiquimod), Aldesleukin, ALECENSA® (Alectinib), Alectinib, Alemtuzumab, ALIMTA® (Pemetrexed Disodium), ALIQOPA® (Copanlisib Hydrochloride), ALKERAN™ for Injection (Melphalan Hydrochloride), ALKERAN™ Tablets (Melphalan), ALOXI® (Palonosetron Hydrochloride), ALUNBRIG® (Brigatinib), AMBOCHLORIN® (Chlorambucil), AMBOCLORIN® (Chlorambucil), Amifostine, Aminolevulinic Acid, Anastrozole, Aprepitant, AREDIA® (Pamidronate Disodium), ARIMIDEX® (Anastrozole), AROMASIN® (Exemestane), ARRANON® (Nelarabine), Arsenic Trioxide, ARZERRA® (Ofatumumab), Asparaginase Erwinia chrysanthemi, Atezolizumab, AVASTIN® (Bevacizumab), Avelumab, Axitinib, Azacitidine, BAVENCIO® (Avelumab), BEACOPP, BECENUM® (Carmustine), BELEODAQ® (Belinostat), Belinostat, Bendamustine Hydrochloride, BEP, BESPONSA® (Inotuzumab Ozogamicin), Bevacizumab, Bexarotene, BEXXAR® (Tositumomab and Iodine I 131 Tositumomab), Bicalutamide, BICNU® (Carmustine), Bleomycin, Blinatumomab, BLINCYTO® (Blinatumomab), Bortezomib, BOSULIF® (Bosutinib), Bosutinib, Brentuximab Vedotin, Brigatinib, BuMel, Busulfan, BUSULFEX® (Busulfan), Cabazitaxel, CABOMETYX® (Cabozantinib-S-Malate), Cabozantinib-S-Malate, CAF, CAMPATH® (Alemtuzumab), CAMPTOSAR® (Irinotecan Hydrochloride), Capecitabine, CAPOX, CARAC® (Fluorouracil—Topical), Carboplatin, CARBOPLATIN-TAXOL, Carfilzomib, CARMUBRIS® (Carmustine), Carmustine, Carmustine Implant, CASODEX® (Bicalutamide), CEM, Ceritinib, CERUBIDINE® (Daunorubicin Hydrochloride), CERVARIX® (Recombinant HPV Bivalent Vaccine), Cetuximab, CEV, Chlorambucil, CHLORAMBUCIL-PREDNISONE, CHOP, Cisplatin, Cladribine, CLAFEN® (Cyclophosphamide), Clofarabine, CLOFAREX® (Clofarabine), CLOLAR® (Clofarabine), CMF, Cobimetinib, COMETRIQ® (Cabozantinib-S-Malate), Copanlisib Hydrochloride, COPDAC, COPP, COPP-ABV, COSMEGEN® (Dactinomycin), COTELLIC® (Cobimetinib), Crizotinib, CVP, Cyclophosphamide, CYFOS® (Ifosfamide), CYRAMZA® (Ramucirumab), Cytarabine, Cytarabine Liposome, CYTOSAR-U® (Cytarabine), CYTOXAN® (Cyclophosphamide), Dabrafenib, Dacarbazine, DACOGEN® (Decitabine), Dactinomycin, Daratumumab, DARZALEX® (Daratumumab), Dasatinib, Daunorubicin Hydrochloride, Daunorubicin Hydrochloride and Cytarabine Liposome, Decitabine, Defibrotide Sodium, DEFITELIO® (Defibrotide Sodium), Degarelix, Denileukin Diftitox, Denosumab, DEPOCYT® (Cytarabine Liposome), Dexamethasone, Dexrazoxane Hydrochloride, Dinutuximab, Docetaxel, DOXIL® (Doxorubicin Hydrochloride Liposome), Doxorubicin Hydrochloride, Doxorubicin Hydrochloride Liposome, DOX-SL® (Doxorubicin Hydrochloride Liposome), DTIC-DOME® (Dacarbazine), Durvalumab, EFUDEX® (Fluorouracil—Topical), ELITEK® (Rasburicase), ELLENCE® (Epirubicin Hydrochloride), Elotuzumab, ELOXATIN® (Oxaliplatin), Eltrombopag Olamine, EMEND® (Aprepitant), EMPLICITI® (Elotuzumab), Enasidenib Mesylate, Enzalutamide, Epirubicin Hydrochloride, EPOCH, ERBITUX® (Cetuximab), Eribulin Mesylate, ERIVEDGE® (Vismodegib), Erlotinib Hydrochloride, ERWINAZE® (Asparaginase Erwinia chrysanthemi), ETHYOL® (Amifostine), Etopophos ETOPOPHOS® (Etoposide Phosphate), Etoposide, Etoposide Phosphate, EVACET® (Doxorubicin Hydrochloride Liposome), Everolimus, EVISTA® (Raloxifene Hydrochloride), EVOMELA® (Melphalan Hydrochloride), Exemestane, 5-FU® (Fluorouracil Injection), 5-FU® (Fluorouracil—Topical), FARESTON® (Toremifene), FARYDAK® (Panobinostat), FASLODEX® (Fulvestrant), FEC, FEMARA® (Letrozole), Filgrastim, FLUDARA® (Fludarabine Phosphate), Fludarabine Phosphate, FLUOROPLEX® (Fluorouracil—Topical), Fluorouracil Injection, Fluorouracil—Topical, Flutamide, FOLEX® (Methotrexate), FOLEX PFS® (Methotrexate), FOLFIRI, FOLFIRI-BEVACIZUMAB, FOLFIRI-CETUXIMAB, FOLFIRINOX, FOLFOX, FOLOTYN® (Pralatrexate), FU-LV, Fulvestrant, GARDASIL® (Recombinant HPV Quadrivalent Vaccine), GARDASIL 9® (Recombinant HPV Nonavalent Vaccine), GAZYVA® (Obinutuzumab), Gefitinib, Gemcitabine Hydrochloride, GEMCITABINE-CISPLATIN, GEMCITABINE-OXALIPLATIN, Gemtuzumab Ozogamicin, GEMZAR® (Gemcitabine Hydrochloride), GILOTRIF® (Afatinib Dimaleate), GLEEVEC® (Imatinib Mesylate), GLIADEL® (Carmustine Implant), GLIADEL WAFER® (Carmustine Implant), Glucarpidase, Goserelin Acetate, HALAVEN® (Eribulin Mesylate), HEMANGEOL® (Propranolol Hydrochloride), HERCEPTIN® (Trastuzumab), HPV Bivalent Vaccine, Recombinant, HPV Nonavalent Vaccine, Recombinant, HPV Quadrivalent Vaccine, Recombinant, HYCAMTIN® (Topotecan Hydrochloride), HYDREA® (Hydroxyurea), Hydroxyurea, Hyper-CVAD, IBRANCE® (Palbociclib), Ibritumomab Tiuxetan, Ibrutinib, ICE, ICLUSIG® (Ponatinib Hydrochloride), IDAMYCIN® (Idarubicin Hydrochloride), Idarubicin Hydrochloride, Idelalisib, IDHIFA® (Enasidenib Mesylate), IFEX® (Ifosfamide), Ifosfamide, IFOSFAMIDUM® (Ifosfamide), IL-2 (Aldesleukin), Imatinib Mesylate, IMBRUVICA® (Ibrutinib), IMFINZI® (Durvalumab), Imiquimod, IMLYGIC® (Talimogene Laherparepvec), INLYTA® (Axitinib), Inotuzumab Ozogamicin, Interferon Alfa-2b, Recombinant, Interleukin-2 (Aldesleukin), INTRON A® (Recombinant Interferon Alfa-2b), Iodine I 1131 Tositumomab and Tositumomab, Ipilimumab, IRESSA® (Gefitinib), Irinotecan Hydrochloride, Irinotecan Hydrochloride Liposome, ISTODAX® (Romidepsin), Ixabepilone, Ixazomib Citrate, IXEMPRA® (Ixabepilone), JAKAFI® (Ruxolitinib Phosphate), JEB, JEVTANA® (Cabazitaxel), KADCYLA® (Ado-Trastuzumab Emtansine), KEOXIFENE® (Raloxifene Hydrochloride), KEPIVANCE® (Palifermin), KEYTRUDA® (Pembrolizumab), KISQALI® (Ribociclib), KYMRIAH® (Tisagenlecleucel), KYPROLIS® (Carfilzomib), Lanreotide Acetate, Lapatinib Ditosylate, LARTRUVO® (Olaratumab), Lenalidomide, Lenvatinib Mesylate, LENVIMA® (Lenvatinib Mesylate), Letrozole, Leucovorin Calcium, LEUKERAN® (Chlorambucil), Leuprolide Acetate, LEUSTATIN® (Cladribine), LEVULAN® (Aminolevulinic Acid), LINFOLIZIN® (Chlorambucil), LIPODOX® (Doxorubicin Hydrochloride Liposome), Lomustine, LONSURF® (Trifluridine and Tipiracil Hydrochloride), LUPRON® (Leuprolide Acetate), LUPRON DEPOT® (Leuprolide Acetate), LUPRON DEPOT-PED® (Leuprolide Acetate), LYNPARZA® (Olaparib), MARQIBO® (Vincristine Sulfate Liposome), MATULANE® (Procarbazine Hydrochloride), Mechlorethamine Hydrochloride, Megestrol Acetate, MEKINIST® (Trametinib), Melphalan, Melphalan Hydrochloride, Mercaptopurine, Mesna, MESNEX® (Mesna), METHAZOLASTONE® (Temozolomide), Methotrexate, METHOTREXATE LPF® (Methotrexate), Methylnaltrexone Bromide, MEXATE® (Methotrexate), MEXATE-AQ® (Methotrexate), Midostaurin, Mitomycin C, Mitoxantrone Hydrochloride, MITOZYTREX® (Mitomycin C), MOPP, MOZOBIL® (Plerixafor), MUSTARGEN® (Mechlorethamine Hydrochloride), MUTAMYCIN® (Mitomycin C), MYLERAN® (Busulfan), MYLOSAR® (Azacitidine), MYLOTARG® (Gemtuzumab Ozogamicin), NANOPARTICLE PACLITAXEL® (Paclitaxel Albumin-stabilized Nanoparticle Formulation), NAVELBINE® (Vinorelbine Tartrate), Necitumumab, Nelarabine, NEOSAR® (Cyclophosphamide), Neratinib Maleate, NERLYNX® (Neratinib Maleate), Netupitant and Palonosetron Hydrochloride, NEULASTA® (Pegfilgrastim), NEUPOGEN® (Filgrastim), NEXAVAR® (Sorafenib Tosylate), NILANDRON® (Nilutamide), Nilotinib, Nilutamide, NINLARO® (Ixazomib Citrate), Niraparib Tosylate Monohydrate, Nivolumab, NOLVADEX® (Tamoxifen Citrate), NPLATE® (Romiplostim), Obinutuzumab, ODOMZO® (Sonidegib), OEPA, Ofatumumab, OFF, Olaparib, Olaratumab, Omacetaxine Mepesuccinate, ONCASPAR® (Pegaspargase), Ondansetron Hydrochloride, ONIVYDE® (Irinotecan Hydrochloride Liposome), ONTAK® (Denileukin Diftitox), OPDIVO® (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, PARAPLAT® (Carboplatin), PARAPLATIN® (Carboplatin), Pazopanib Hydrochloride, PCV, PEB, Pegaspargase, Pegfilgrastim, Peginterferon Alfa-2b, PEG-INTRON® (Peginterferon Alfa-2b), Pembrolizumab, Pemetrexed Disodium, PERJETA® (Pertuzumab), Pertuzumab, PLATINOL® (Cisplatin), PLATINOL-AQ® (Cisplatin), Plerixafor, Pomalidomide, POMALYST® (Pomalidomide), Ponatinib Hydrochloride, PORTRAZZA® (Necitumumab), Pralatrexate, Prednisone, Procarbazine Hydrochloride, PROLEUKIN® (Aldesleukin), PROLIA® (Denosumab), PROMACTA® (Eltrombopag Olamine), Propranolol Hydrochloride, PROVENGE® (Sipuleucel-T), PURINETHOL® (Mercaptopurine), PURIXAN® (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, RELISTOR® (Methylnaltrexone Bromide), R-EPOCH, REVLIMID® (Lenalidomide), RHEUMATREX® (Methotrexate), Ribociclib, R-ICE, RITUXAN® (Rituximab), RITUXAN HYCELA® (Rituximab and Hyaluronidase Human), Rituximab, Rituximab and, Hyaluronidase Human, Rolapitant Hydrochloride, Romidepsin, Romiplostim, RUBIDOMYCIN® (Daunorubicin Hydrochloride), RUBRACA® (Rucaparib Camsylate), Rucaparib Camsylate, Ruxolitinib Phosphate, RYDAPT® (Midostaurin), Sclerosol Intrapleural Aerosol (Talc), Siltuximab, Sipuleucel-T, SOMATULINE DEPOT® (Lanreotide Acetate), Sonidegib, Sorafenib Tosylate, SPRYCEL® (Dasatinib), STANFORD V, Sterile Talc Powder (Talc), STERITALC® (Talc), STIVARGA® (Regorafenib), Sunitinib Malate, SUTENT® (Sunitinib Malate), SYLATRON® (Peginterferon Alfa-2b), SYLVANT® (Siltuximab), Synribo SYNRIBO® (Omacetaxine Mepesuccinate), TABLOID® (Thioguanine), TAC, TAFINLAR® (Dabrafenib), TAGRISSO® (Osimertinib), Talc, Talimogene Laherparepvec, Tamoxifen Citrate, TARABINE PFS® (Cytarabine), TARCEVA® (Erlotinib Hydrochloride), TARGRETIN® (Bexarotene), TASIGNA® (Nilotinib), TAXOL® (Paclitaxel), TAXOTERE® (Docetaxel), TECENTRIQ® (Atezolizumab), TEMODAR® (Temozolomide), Temozolomide, Temsirolimus, Thalidomide, THALOMID® (Thalidomide), Thioguanine, Thiotepa, Tisagenlecleucel, TOLAK® (Fluorouracil—Topical), Topotecan Hydrochloride, Toremifene, TORISEL® (Temsirolimus), Tositumomab and Iodine I 131 Tositumomab, TOTECT® (Dexrazoxane Hydrochloride), TPF, Trabectedin, Trametinib, Trastuzumab, TREANDA® (Bendamustine Hydrochloride), Trifluridine and Tipiracil Hydrochloride, TRISENOX® (Arsenic Trioxide), TYKERB® (Lapatinib Ditosylate), UNITUXIN® (Dinutuximab), Uridine Triacetate, VAC, Vandetanib, VAMP, VARUBI® (Rolapitant Hydrochloride), VECTIBIX® (Panitumumab), VeIP, VELBAN® (Vinblastine Sulfate), VELCADE® (Bortezomib), VELSAR® (Vinblastine Sulfate), Vemurafenib, VENCLEXTA® (Venetoclax), Venetoclax, VERZENIO® (Abemaciclib), VIADUR® (Leuprolide Acetate), VIDAZA® (Azacitidine), Vinblastine Sulfate, VINCASAR PFS® (Vincristine Sulfate), Vincristine Sulfate, Vincristine Sulfate Liposome, Vinorelbine Tartrate, VIP, Vismodegib, VISTOGARD® (Uridine Triacetate), VORAXAZE® (Glucarpidase), Vorinostat, VOTRIENT® (Pazopanib Hydrochloride), VYXEOS® (Daunorubicin Hydrochloride and Cytarabine Liposome), WELLCOVORIN® (Leucovorin Calcium), XALKORI® (Crizotinib), XELODA® (Capecitabine), XELIRI, XELOX, XGEVA® (Denosumab), XOFIGO® (Radium 223 Dichloride), XTANDI® (Enzalutamide), YERVOY® (Ipilimumab), YONDELIS® (Trabectedin), ZALTRAP® (Ziv-Aflibercept), ZARXIO® (Filgrastim), ZEJULA® (Niraparib Tosylate Monohydrate), ZELBORAF® (Vemurafenib), ZEVALIN® (Ibritumomab Tiuxetan), ZINECARD® (Dexrazoxane Hydrochloride), Ziv-Aflibercept, ZOFRAN® (Ondansetron Hydrochloride), ZOLADEX® (Goserelin Acetate), Zoledronic Acid, ZOLINZA® (Vorinostat), ZOMETA® (Zoledronic Acid), ZYDELIG® (Idelalisib), ZYKADIA® (Ceritinib), and/or ZYTIGA® (Abiraterone Acetate). The treatment methods can include or further include checkpoint inhibitors including, but are not limited to antibodies that block PD-1 (such as, for example, lambrolizumab, OPDIVO® (Nivolumab), BMS-936558, MDX1106, KEYTRUDA® (pembrolizumab), pidilizumab, cemiplimab, CT-011, MK-3475), PD-L1 (such as, for example, TECENTRIQ® (Atezolizumab), BAVENCIO® (Avelumab), IMFINZI® (Durvalumab), MDX-1105 (BMS-936559), MPDL3280A, or MSB0010718C), PD-L2 (such as, for example, rHIgM12B7), CTLA-4 (such as, for example, YERVOY® (ipilimumab) (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313 M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep). In one aspect, the CD4+ T cell mediated therapy can comprise adoptive cell therapies, and CAR T therapies.

The combination of fucose and an anti-cancer agent or immune checkpoint inhibitor can be formulated in the same composition or separately. Where separate, the fucose can be administered before, after, or concurrently with the anti-cancer agent. Administration of fucose can be accomplished prophylactically or therapeutically.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### C. EXAMPLES

### 1. Example 1: Brain Met-Associated Fibroblasts Drive Breast Cancer Invasive Capacity by Secreting Fucosylated PVR/CD155

Breast Cancer is the second leading cause of brain metastasis, it accounts for 30% of total brain metastasis. Within Breast Cancer, patients with TNBC have the highest incidence of brain metastasis, and once diagnosed with brain metastasis the survival is extremely poor. Current treatment strategies for breast cancer brain metastasis (BCBM) are limited to either WBRT or surgical resection and at systemic level its either targeted, hormonal or chemotherapy. Most of the times, patients suffer severe side effects with neurological problems and quality of life is reduces. Hence, a better biological understanding of BCBM is crucial right now so that we can bring in new successful treatment strategies101 and biomarkers.

One of the most prominent stromal cells in breast cancer TME are cancer-associated fibroblasts (CAFs). CAFs are known to influence almost all hallmarks of cancer including metastasis. CAFs have been isolated from the brains of patients with BCBM and these brain-metCAFs secrete chemokines and growth factors that enhance BCBM. However, these studies are extremely limited, only handful and definitive molecular mechanisms is not known.

Fucosylation is a type of post-translational modification, where proteins are decorated with dietary sugar L-fucose. It occurs in Golgi-ER compartment via 13 different FUTs and the fucosylated proteins are membrane bound receptors or secreted proteins. Some of the well characterized fucosylated receptors include—EGFR, NOTCH, TGFB. Altered fucosylation impacts proliferation, migration and tumor: immune interactions.

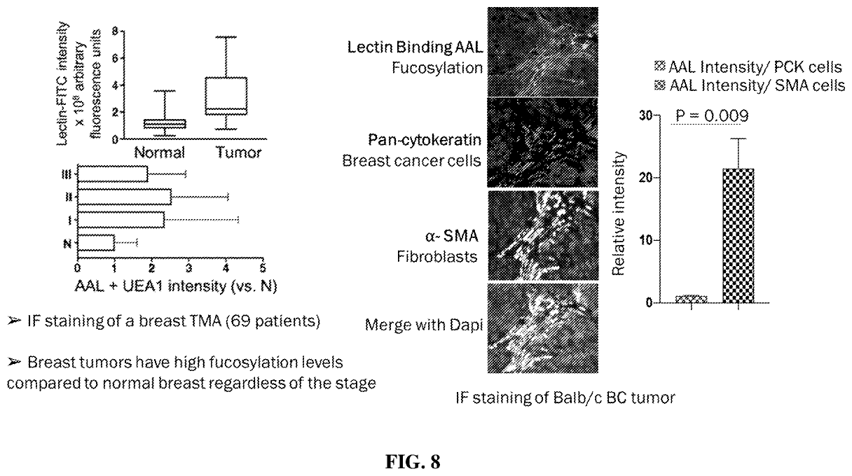

Breast cancer have higher global fucosylation level as compared to Normal tissue regardless of the stages of the patients. Interestingly, when IF staining of breast cancer mouse tissue was performed, we observed that the high fucosylation signal shown here by lectin binding AAL is coming from fibroblast like SMA positive cells, it's almost 20-fold higher than from Pan CK cells. This prompted us to ask if CAFs use fucosylated substrate in paracrine crosstalk with BC cells (FIG. 8).

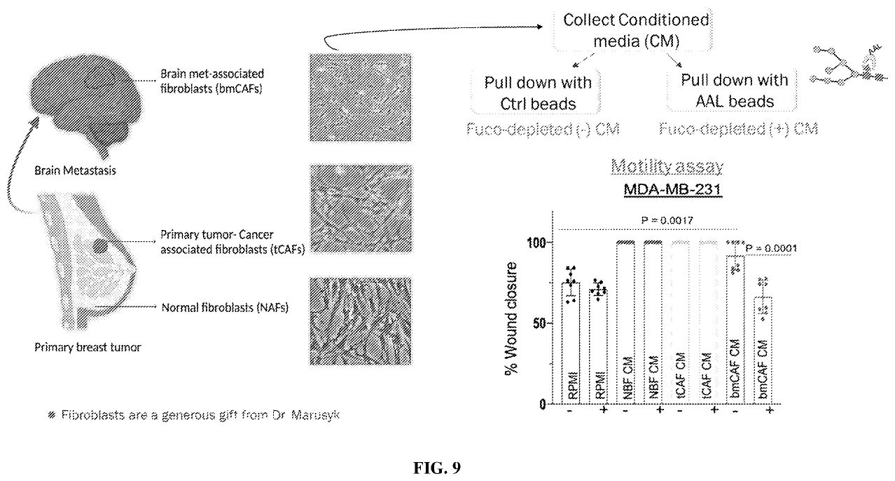

We obtained three types of fibroblasts, brain-met associated fibroblasts, primary tumor CAFs and normal fibroblasts (NAFs). By immunoblotting we observed that they express varying levels of fucosylated proteins. To address if they secrete fucosylated proteins and if they are required for BC tumorigenicity, we collected conditioned media from them and CM was depleted of fucosylated proteins using lectin binding AAL beads. On a 2D motility assay we observed that CM from NAF or tCAFs enhance the migration of metastatic TNBC cells regardless of fucosylated proteins present or absent in the media. Interestingly, we observed that when fucosylated proteins were depleted from the bmCAFs CM the migration effect was blunted. We observed similar fucosylated protein dependent effect in a proliferation assay as well. (FIG. 9)

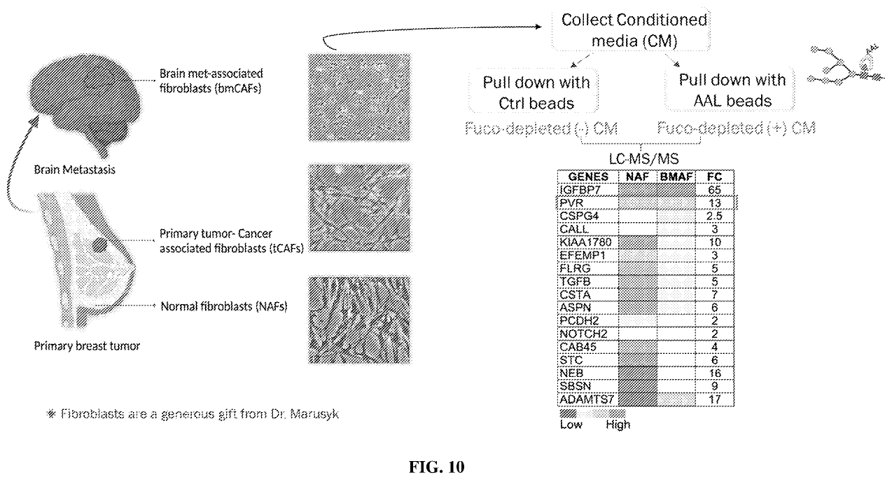

To understand what are the fucosylated proteins secreted by bmCAFs that are potentially driving this phenotype, we identified proteins bound to these beads by LC-MS/MS (FIG. 10). By several filtering criteria, we came up with a list of proteins and one of the proteins caught our attention—PVR (poliovirus receptor), it is one of the most abundant secreted proteins within bmCAFs and one of the highly altered as compared to NAFs.

To address if PVR is directly fucosylated, we performed lectin-mediated PLA. When the fucose is close to PVR, only then will we see the punctae, which is shown in FIG. 11. More importantly, when we treat these cells with Brefeldin A (which blocks the secretion of proteins), we observed increased punctae-fucosylated PVR indicating that the secreted PVR is fucosylated. We further confirmed the fucosylation of PVR by multiple ways and upregulation of PVR in multiple patient derived bmCAFs.

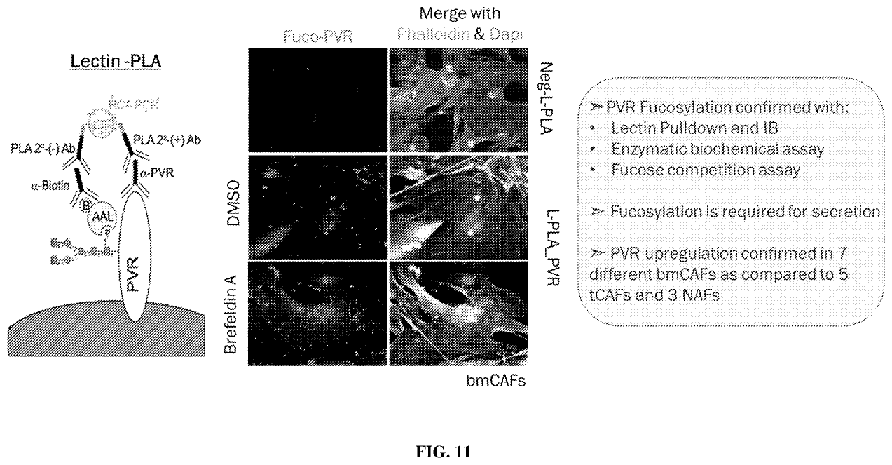

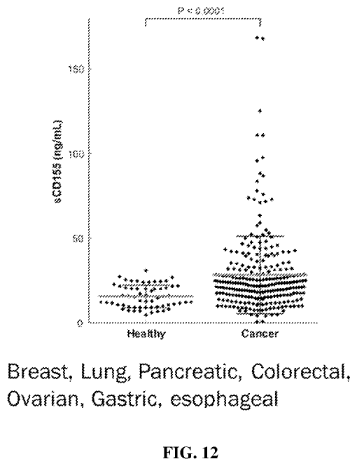

PVR is a cell surface adhesion molecule and acts as an immunomodulatory receptor, ligand for TIGIT and CD226 receptors. It is overexpressed in multiple cancers and can influence proliferation, migration, and adhesion. It also acts as an immunomodulatory receptor, can have both Pro-tumor as well as anti-tumor immunity function. It has multiple facets. It can exist in 2 isoforms: soluble and transmembrane. Soluble PVR in detected in serum of patients with multiple cancer types and Serum PVR is higher in cancer patients than normal patients (FIG. 12)

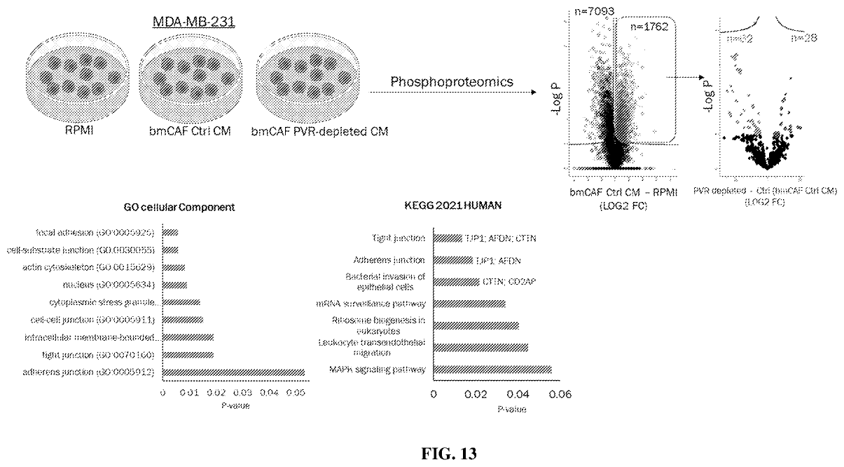

To understand how secreted PVR is influencing BC cell biology, we performed a phosphoproteomics experiment (FIG. 13). TNBC cells were treated with regular media, Ctrl CM from bmCAFs or PVR-immunodepleted CM. Approximately 1800 phosphopeptides were enriched with the ctrl CM and among them 62 proteins were downregulated when PVR was absent from the CM. Pathway analysis revealed that those 62 proteins were implicated in tight junction, adherens junction and focal adhesion regulation. To sum-up, what this is telling is: secreted PVR from bmCAFs drive BC cells migration/invasion capacity by regulation tight/adherens junction proteins.

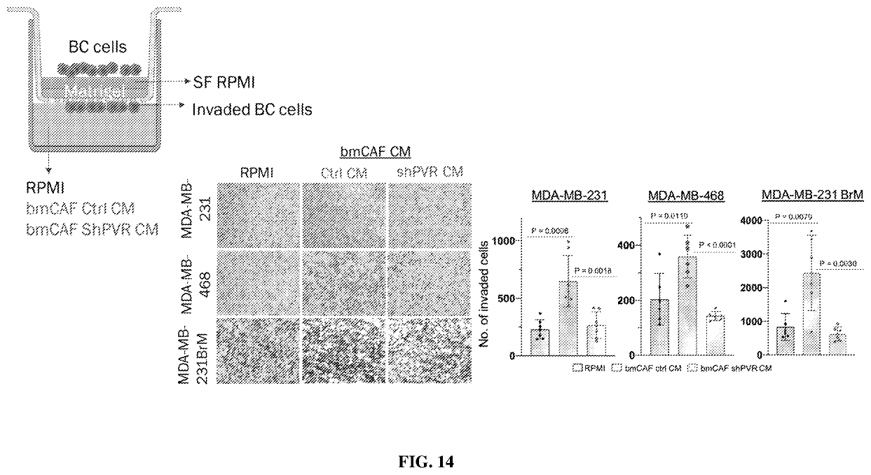

In similar line with phosphoproteomics data, in a matrigel invasion assay (FIG. 14) we observed that the TNBC cells invaded more when CM from bmCAFs was used as a chemoattractant and when the Cm was devoid of PVR there was reduction in the invaded cells. We observed similar effect in a 2D migration assay as well. These observations hold true in multiple TNBC cells lines and a brain homing TNBC 231 cells as well.

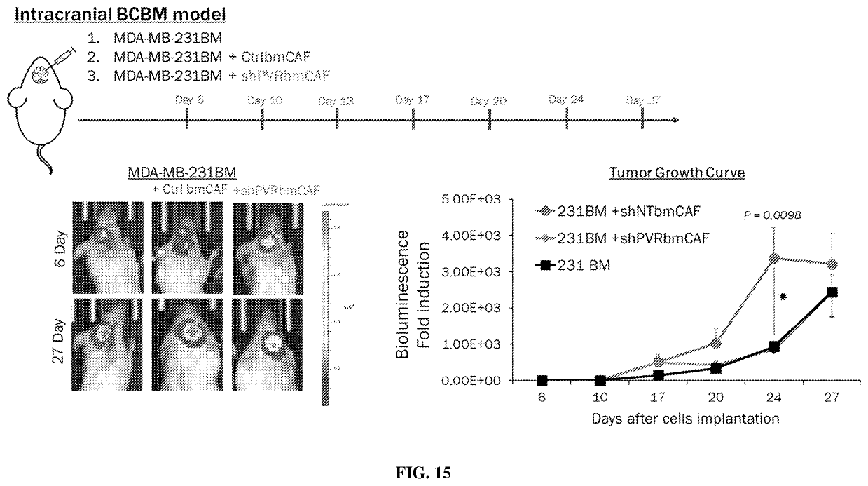

To replicate this observation in-vivo, we co-cultured BC cells with bmCAFs or PVR knock-down bmCAFs and performed an intracranial injection (FIG. 15). We observed that the bmCAFs provided an overall survival advantage to BC cells which was blunted when the BC cells were together with PVR knockdown bmCAFs.

### 2. Example 2: Brain Metastasis-Associated Fibroblasts Secrete Fucosylated PVR/C155 that Induces Breast Cancer Invasion

Breast cancer (BC) is the second most common source of brain metastasis (BM), which occurs in 10%-30% of metastatic BC (MBC) patients. Patients with triple-negative breast cancer (TNBC), the most aggressive subtype of BC, exhibit the highest BM incidence rates and poorest survival outcomes upon diagnosis with BM. As brain screening for MBC patients is not recommended in the US National Comprehensive Cancer Network and European Society for Medical Oncology guidelines, effective early detection remains a challenge: most BC patients are diagnosed with BM after presentation of neurological symptoms (well after dissemination of cancer cells within the brain). Effective therapies are also lacking. Since the blood-brain barrier (BBB) is a major obstacle for the effective delivery of many drugs into the brain, local treatments including surgery, stereotactic radiation therapy, and whole-brain radiation therapy are currently considered as gold-standard treatments for BCBM. Unfortunately, these approaches often result in impaired neurocognitive function and significantly reduced quality of life. Thus, new early-detection/screening modalities and more effective targeted/personalized therapies with fewer comorbidities are urgently needed, particularly for high-risk BC patients.

Increasing studies have identified cancer-associated fibroblasts (CAFs) as major players within the tumor microenvironment (TME) of breast and other cancer types that promote tumor cell migration and invasion. Thus, targeting CAFs has been an attractive and promising strategy for cancer treatment. Significant advances have been made in CAF-targeted therapies that generally aim to (1) directly or indirectly deplete CAFs using antibodies or vaccines or (2) normalize or reprogram CAFs to a non-pathological, benign state. However, none of these approaches have translated from pre-clinical models to therapeutic efficacy in human trials. Thus, the delineation of alternative approaches (i.e., targeted inhibition of CAF intrinsic or paracrine signaling mechanisms that more effectively block CAF-mediated tumorigenesis) in humans is needed.

While CAFs and their protumoral effects have been demonstrated in primary breast tumors, CAFs have been presumed absent in BCBM, as fibroblasts do not reside in the healthy brain. However, CAFs have been isolated from BCBM tissues, suggesting that they either traffic with cancer cells from primary tumors or transdifferentiate from brain-resident stromal cells (e.g., pericytes or mesenchymal stem cells). Notably, these brain-metastasis-associated CAFs (bmCAFs) have been found to promote metastatic colonization by producing high levels of chemokines (e.g., CXCL12 and CXCL16) that enhance the migration of patient-derived cancer cells, induce BBB permeability, and provide survival, proliferative, and invasive advantages to tumor cells. However, definitive underlying and targetable mechanisms are unknown. More in-depth studies elucidating CAF-mediated BCBM biology are crucial and anticipated to result in new innovative diagnostic and therapeutic approaches for targeting CAF-mediated mechanisms to suppress BCBM.

Emerging studies have highlighted mechanistic roles for fucosylation in the pathogenesis of multiple cancer types, including those within the brain. Fucosylation is the post-translational conjugation of the dietary sugar L-fucose (L-fuc) onto N- or O-linked oligosaccharide chains on glycoproteins or onto glycolipids. Fucosylation begins with cytoplasmic synthesis of GDP-L-fucose (GDP-L-fuc) that is transported into the ER/Golgi by the transporters SLC35C1/2, where it is conjugated onto proteoglycans by fucosyltransferases (FUTs). Thirteen FUTs catalyze structurally distinct fucose linkages in humans, and the aberrant up- and downregulation of individual FUTs is associated with tumorigenesis. The activity of key receptors (e.g., integrins, epidermal growth factor receptor, transforming growth factor receptor (3, and Notch) are fine-tuned by fucosylation in terms of ligand binding, dimerization, and signaling capacities. Profiling of cancer patient sera for altered glycosylation-fucosylation states/levels of secreted proteins is a promising new diagnostic approach; increased serum levels of L-fuc and fucosylated proteins correlate with BC progression. Notably, altered abundance of L-fuc and N- and O-fucosylated glycans have been reported in brain cancers including glioblastoma (GBM), consistent with pathological contributions. However, how aberrantly fucosylated proteins alter tumor-stromal interactions (e.g., tumor:CAF interactions) to promote progression of tumors in the brain is not known.

Here, we report that compared with normal and primary tumor fibroblasts, patient-derived bmCAFs uniquely secrete fucosylated proteins that induce migration/invasion of TNBC cells. We identified polio virus receptor (PVR/CD155) as a highly bmCAF-fucosylated/secreted protein that potently drives BC cell migration/invasion. Consistent with the hypoxic environment in the brain parenchyma, our studies delineate that fucosylation of PVR is facilitated by FUT11 that is directly transcriptionally upregulated by hypoxia-inducible factor 1α (HIF1α). Intracranial mouse modeling of BCBM using a brain-homing BC cell line reveals the crucial roles played by secreted fucosylated PVR and FUT11 in CAF-mediated invasion and expansion of BCBM in the brain. Analysis of BCBM patient tissues reveals that fucosylated PVR is enhanced in bmCAF compared to BC populations. Together, our findings highlight a fucosylation- and hypoxia-regulated mechanism underlying bmCAF-mediated BCBM pathogenesis.
a) Results
   (1) BC CAFs exhibit high fucosylation levels that correlate with metastasis

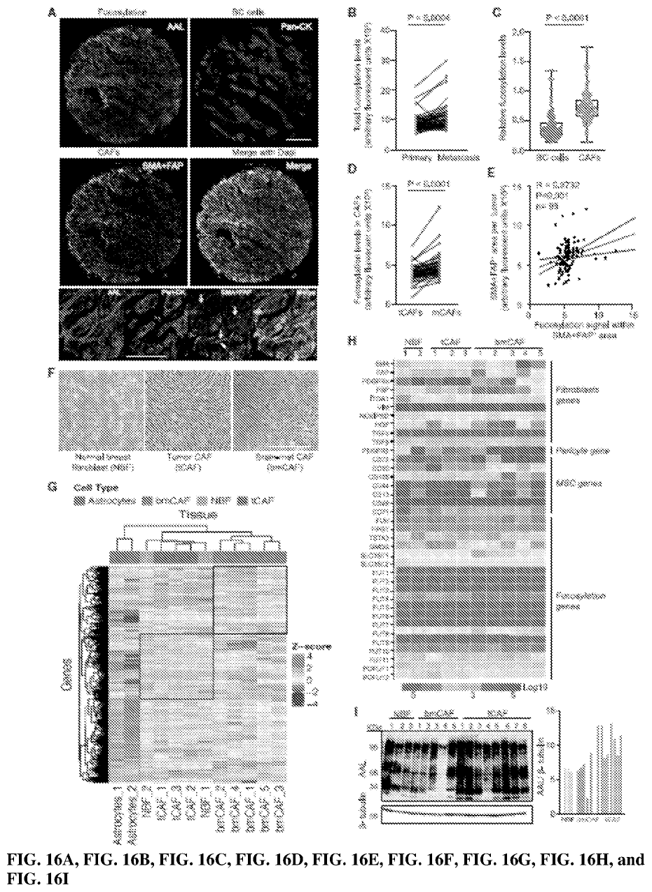

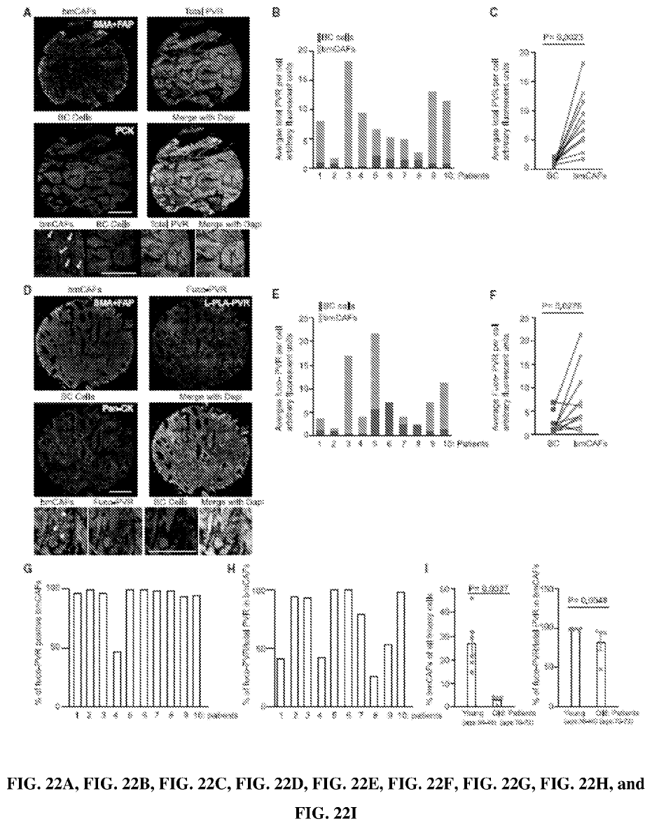

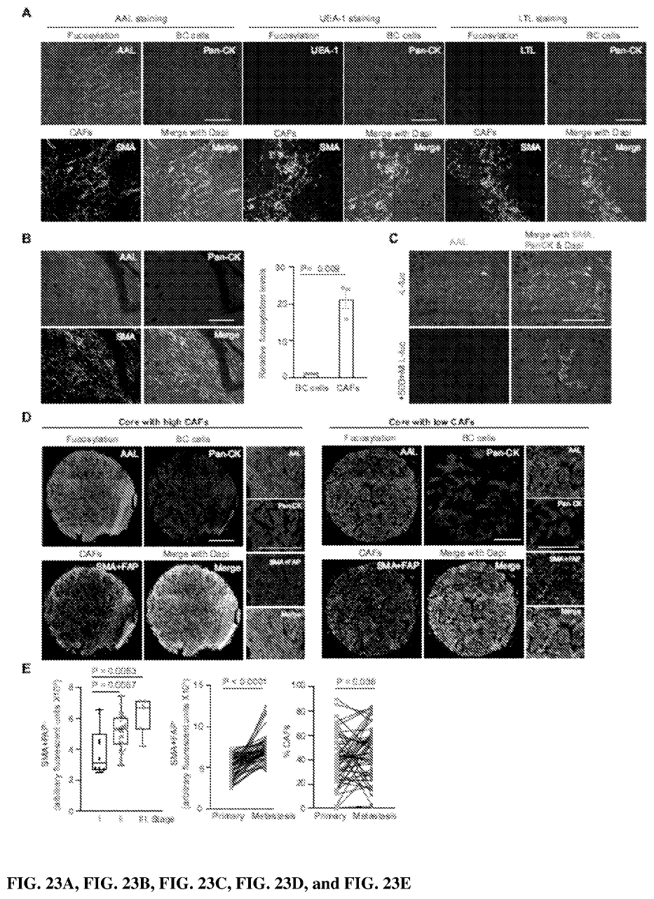

The importance of fucosylation in cellular signaling and tumor progression continues to be defined in emerging studies. However, our understanding of the roles of fucosylation in key cell type(s) within the TME and their contributions to tumorigenesis remains limited. To understand the dynamics of fucosylation within the TME of breast tumors, we performed immunofluorescent (IF) staining of MDA-MB-231 tumor xenograft sections using three different types of lectins that recognize different fucosylation structural linkages: AAL: α(1,2), α(1,3), α(1,4), and α(1,6); UEA-1: α(1,2); and LTL: α(1,3) and α(1,4). AAL exhibited the highest signals within the tumors compared to UEA-1 and LTL (FIG. 23A). More detailed inspection of the intratumoral IF staining revealed that SMA⁺ cells exhibited ~20-fold higher levels of AAL-recognized fucosylation compared to pan-cytokeratin (Pan-CK)⁺ BC cells (FIGS. 23A and 23B). To confirm the specificity of AAL for immunostaining of fucosylated proteins, we performed a control L-fuc wash during AAL staining of the tumors, which abolished all AAL IF signals (FIG. 23C). We subsequently assessed the correlation between AAL-recognized fucosylation and CAFs in human BC by multiplexed IF staining and single-cell segmented imaging analysis of a 50-patient breast tumor microarray (TMA), which contained one primary and one metastatic core biopsy pair per patient (FIGS. 16A and 23D). Total AAL-recognized fucosylation levels were increased in metastatic compared to patient-matched primary tumors (FIG. 16B). Intriguingly, we discovered that relative AAL-recognized fucosylation levels in CAFs were significantly higher than in BC cells (FIG. 16C), and metastatic CAFs (mCAFs) exhibited significantly higher fucosylation levels compared to matched tumor CAFs (tCAFs) (FIG. 16D). Notably, Pearson analysis revealed a correlation (r=0.3732) between the number of CAFs and their level of fucosylation (FIG. 16E). Moreover, intratumoral CAF populations (absolute and percentage of total tumor cells) correlated with increased clinical staging and metastasis (FIG. 23E), consistent with previous reports, suggesting that increased intratumoral fucosylation in breast tumors is attributed to CAF-specific fucosylation that increases with intratumoral CAF density during progression. These observations suggest that CAF-specific fucosylation plays an essential role in BC progression. However, why CAFs exhibit such high fucosylation levels and whether and how CAF fucosylated proteins might impact BC biology was unclear.

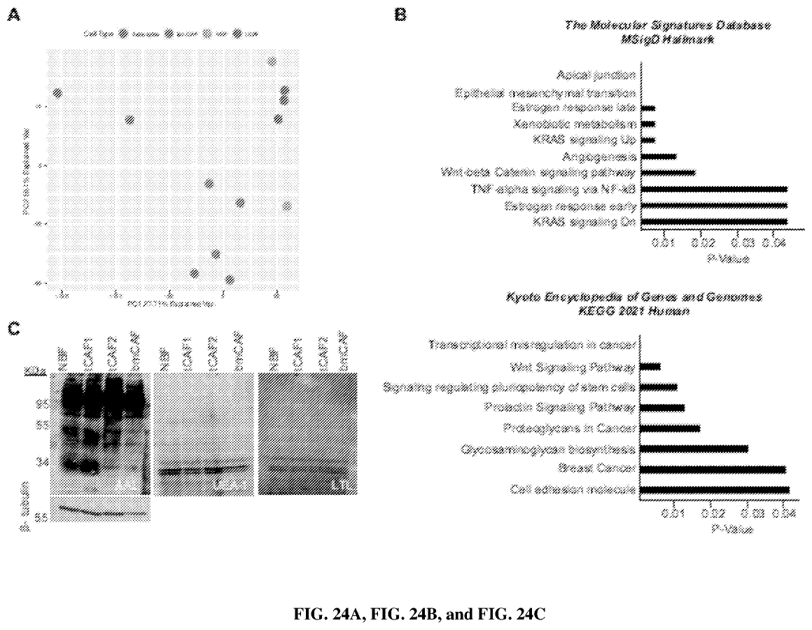

To investigate whether fucosylated proteins from patient-derived CAFs contribute to BC progression, we obtained normal breast fibroblasts (NBFs), primary breast tumor CAFs (tCAFs), and BC brain metastasis CAFs (bmCAFs) (FIG. 16F) from our Moffitt collaborator and co-author A. M. RNA sequencing (RNA-seq) data from Marusyk et al. demonstrate that NBF, tCAFs, and bmCAFs have gene set clusters that are both distinct and in common, suggesting that all three are phenotypically fibroblasts that are distinct from each other depending on tissue of origin and are distinct from astrocytes that are commonly found in the brain (FIG. 16G). Principal component analysis (PCA) further confirmed that bmCAFs cluster distinctly from tCAFs and astrocytes (FIG. 24A). Differential gene expression analysis revealed 52 and 206 genes in bmCAFs that were upregulated and downregulated, respectively, by ≥2.5-fold (adjusted p value ≤0.05) compared to tCAFs. Pathway analysis identified apical junction, epithelial mesenchymal transition (EMT), and Wnt signaling as the top three pathways reflected by these genes (FIG. 24B). Subcluster analysis of the RNA-seq data revealed that these fibroblasts co-express canonical fibroblast markers including SMA, FAP, PDGFRα, TGFα, TGFβ, ITGA1, VIM, and HGF, mesenchymal stem cell genes, pericyte marker PDGFRβ, and all 19 fucosylation machinery genes (FIG. 16H). To characterize global intracellular fucosylation levels, we analyzed NBF, tCAF, and bmCAF lysates by AAL, UEA-1, and LTL immunoblot (IB). We observed that the strongest global fucosylation signal, covering a wide range of proteins from low to high molecular weight, was detected by AAL lectin (FIG. 24C). Among all three types of fibroblasts, we observed a trend of higher AAL-recognized fucosylation in tCAFs compared to NBFs and bmCAFs (FIG. 16I). Taken together, these results indicate that BC CAFs exhibit higher fucosylation levels that correlate with higher tumor staging and metastasis, underscoring potential roles of CAF-driven fucosylated proteins in BC tumor progression.
(2) Secreted fucosylated proteins from bmCAFs drive BC cell migration

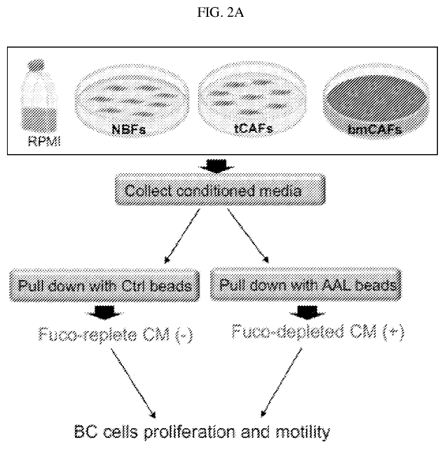

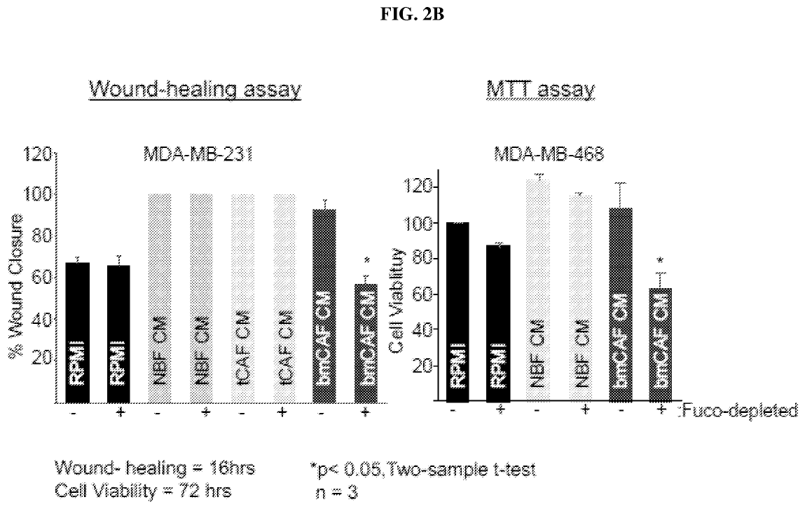

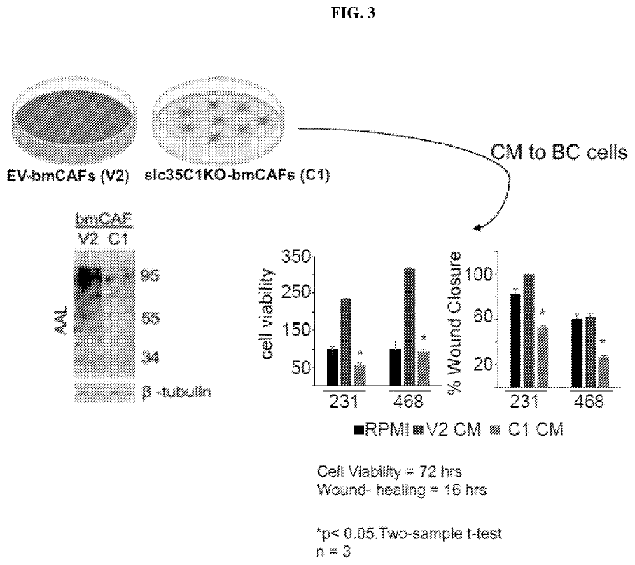

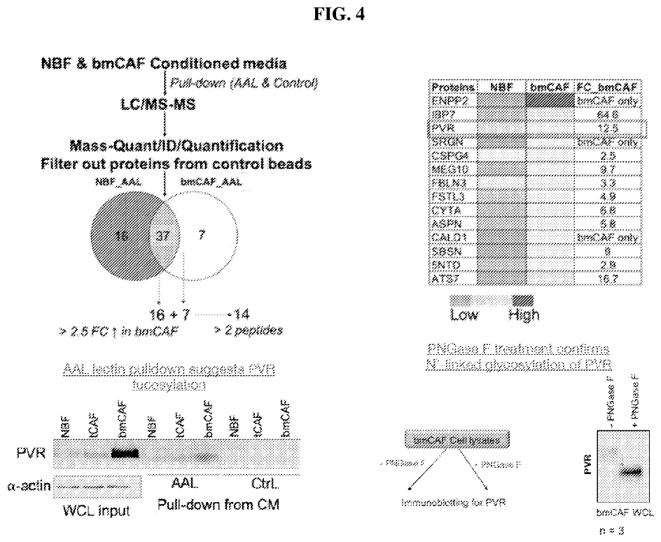

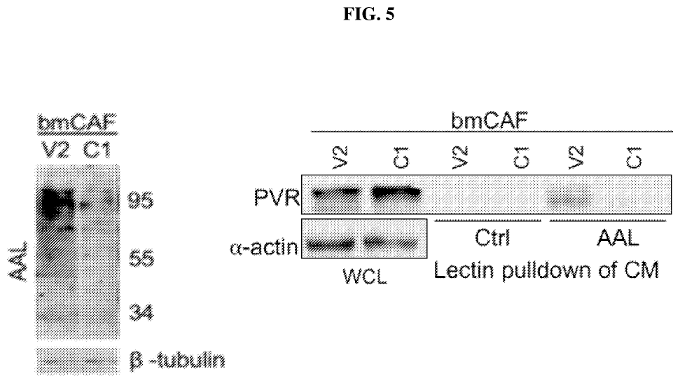

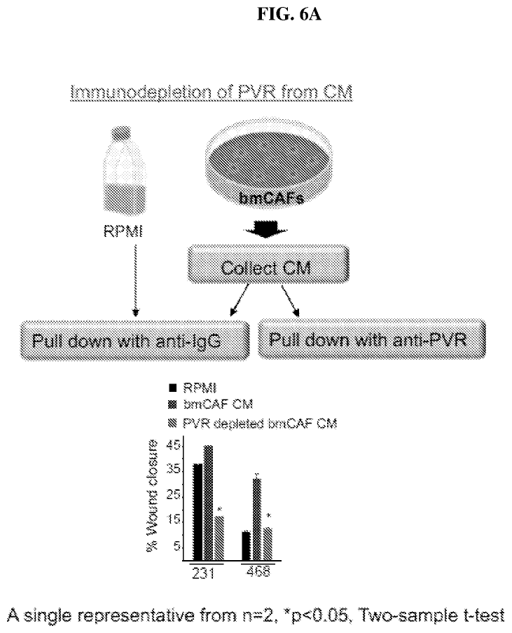

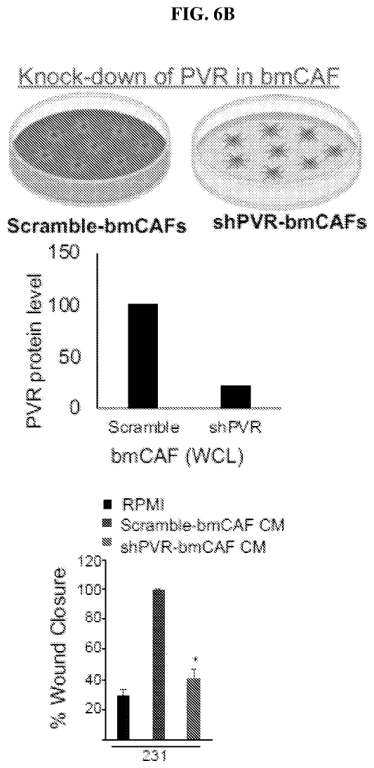

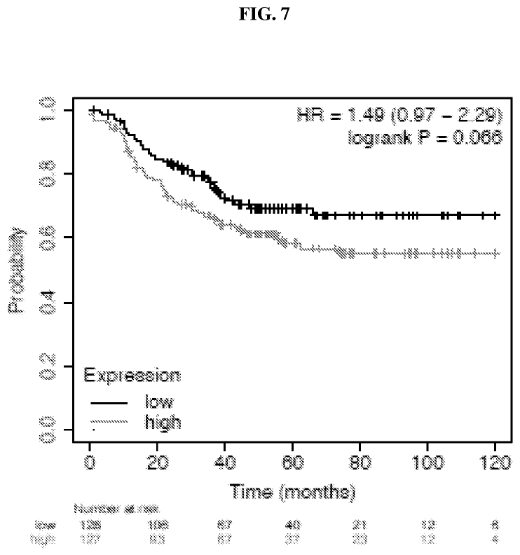

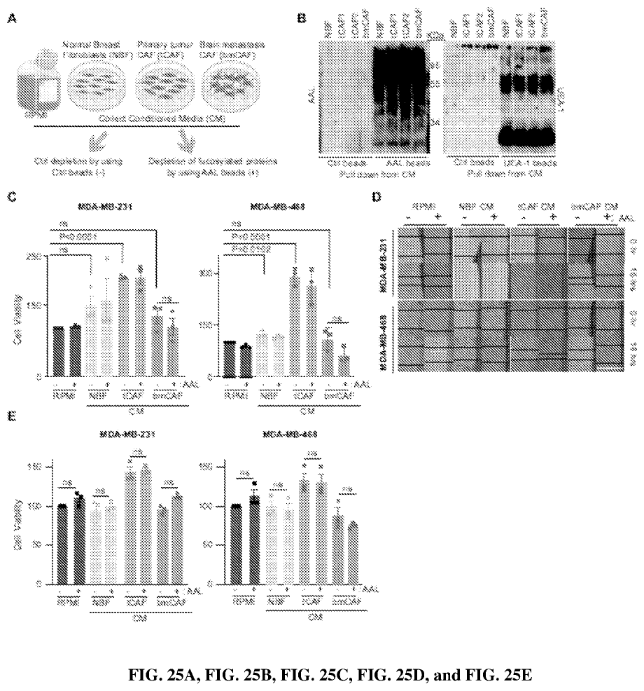

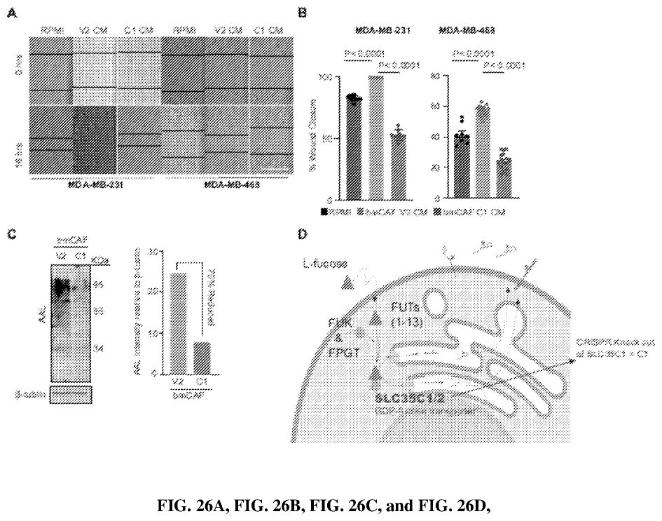

As CAFs are known to secrete tumorigenic growth factors, chemokines, and cytokines into the TME, we sought to determine whether CAFs secrete fucosylated proteins that contribute to BC pathogenesis. To this end, we assessed the proliferation and motility of TNBC cells cultured with NBF-, tCAF-, or bmCAF-derived conditioned medium (CM) that was depleted or not of fucosylated proteins (FIG. 25A). We pre-depleted CM of fucosylated proteins using AAL and UEA-1 beads; we used AAL beads thereafter, as AAL depleted more fucosylated proteins ranging from high to low molecular weight compared with UEA-1 (FIG. 25B). Compared to RPMI and regardless of fucosylated protein content, tCAF CM significantly stimulated the proliferation of MDA-MB-231 and MDA-MB-468 cells, whereas NBF and bmCAF CM did not (FIG. 25C). The motility of BC cells was also increased significantly by NBF and tCAF CM, regardless of fucosylated protein content. Conversely, only fucosylated protein-replete bmCAF CM increased BC motility; depletion of fucosylated proteins abolished bmCAF CM-stimulated motility (FIGS. 2A and 25D). The observed differences were attributed solely to the motility effects, since we did not observe any proliferation differences between the conditions at this time point (FIG. 25E). Similar abrogation of bmCAF CM-stimulated motility (FIGS. 26A and 26B) was observed when BC cells were treated with CM from fucosylation-deficient bmCAFs (FIG. 26C) that were generated by CRISPR knockout of the GDP-L-fuc transporter SLC35C1 (FIG. 26D). Together, these data demonstrate that bmCAFs secrete fucosylated proteins that are crucial for stimulation of motility in BC cells.
(3) Soluble PVR is a fucosylated protein that is highly secreted from bmCAFs

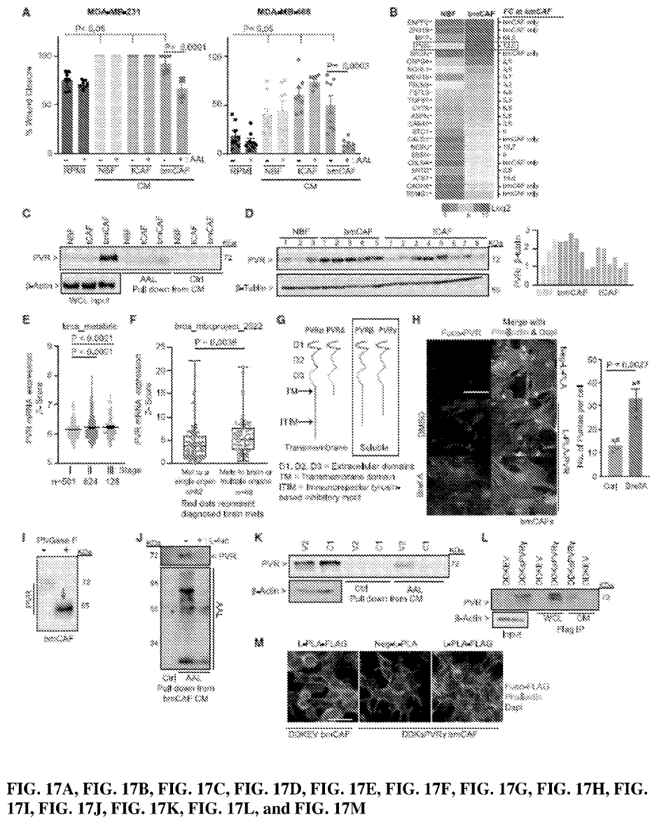

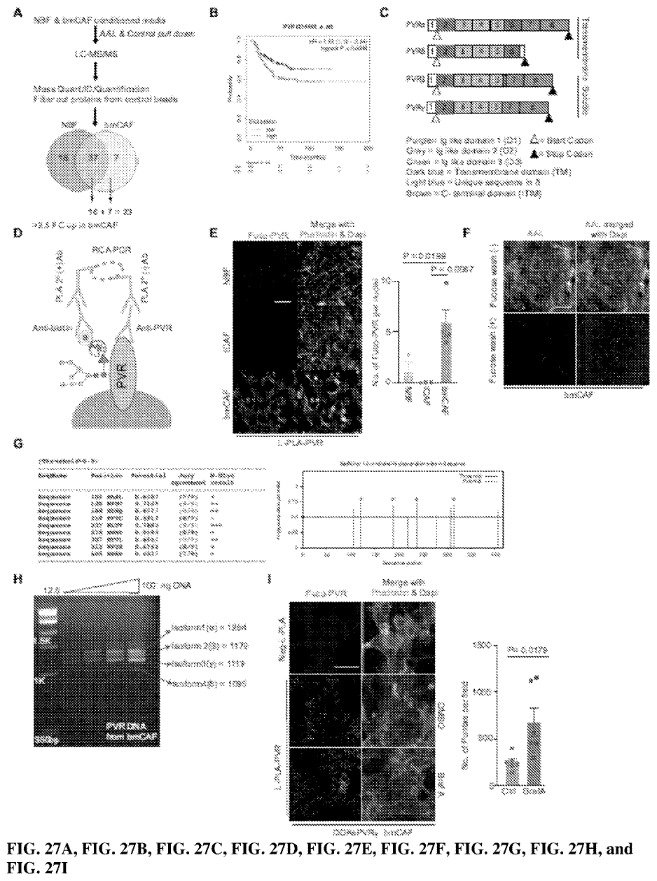

To identify the specific bmCAF-secreted fucosylated proteins that may stimulate the migration of TNBC cells, we performed comparative mass spectrometry (MS) profiling of fucosylated proteins isolated from NBF and bmCAF CM using agarose-bound AAL lectin. Twenty-three putatively fucosylated proteins were identified by liquid chromatography-tandem MS (LC-MS/MS) as exclusively or significantly secreted by bmCAFs (FIGS. 27A and 17B). Lectin pull-down immunoblot (LPD-IB) analysis of the top five hits successfully validated PVR as secreted and fucosylated by bmCAFs compared to NBFs and tCAFs (FIG. 17C). Comparison of multiple patient-derived bmCAFs, NBFs, and tCAFs revealed that PVR protein levels was generally increased in bmCAFs (FIG. 17D). Analysis of BC patient cohort expression datasets (brca_metabric [European Genome-phenome Archiv][EGA]: EGAS00001001753] and brca_mbcproject) further substantiated that PVR levels significantly correlate with increasing tumor staging and are strikingly higher in patients with multi-organ metastasis-who are more likely to develop BM-compared to those with single-organ metastasis (FIGS. 17E and 17F). Consistent with these observations, Kaplan-Meier analysis using KM plotter revealed that increased PVR expression is associated with poorer overall survival in TNBC patients (FIG. 27B).

Poliovirus receptor is a cell surface type I glycoprotein, the mRNA transcript of which can undergo alternative splicing, potentially resulting in two transmembrane (a and 6) and two soluble (β and γ) isoforms (FIG. 27C). The longest is a transmembrane isoform comprising three extracellular, one transmembrane, and one intracellular immunoreceptor tyrosine-based inhibitory motif (ITIM)-containing domains. The second transmembrane isoform only lacks the ITIM domain. The two soluble isoforms lack a complete transmembrane domain and differ from each other in the length of the ITIM domain (FIG. 17G). Transmembrane PVR (tPVR) exhibits increased expression in tumor cells, which can promote tumor progression by facilitating tumor cell-matrix and cell-cell interactions, as well as modulating anti-tumor natural killer (NK) and T cell responses. In contrast, studies focusing on soluble PVR (sPVR) and its pathological contributions to cancer have been lacking, although the presence of sPVR has been reported in the serum of patients with cancers, including glioblastoma. Moreover, the fucosylation status of PVR and regulation of its secretion from CAFs has not yet been reported.

To further characterize the fucosylation and secretion of sPVR, we utilized the lectin-mediated proximity ligation assay (L-PLA), a technique that was developed in our laboratory (FIG. 27D), to immunofluorescently visualize fucosylated PVR. We confirmed the direct fucosylation of PVR in bmCAFs but not in NBFs or tCAFs by using L-PLA (FIG. 27E). More importantly, treatment of the bmCAFs with brefeldin A (BrefA), an inhibitor of the secretory pathway, increased the amount of fucosylated PVR (fuco-PVR) puncta contained within the cells, suggesting that fuco-PVR is secreted from bmCAFs (FIG. 17H). As a control to confirm the specificity of AAL for L-fuc, we performed an L-fuc wash during AAL staining of coverslip-grown bmCAFs, which resulted in complete loss of AAL IF signal (FIG. 27F). To further delineate the site-specific fucosylation of PVR, we used NetNGlyc and NetOGlyc software, which predicted four potential N-linked glycosylations of PVR (FIG. 27G). To test for N-glycosylation of PVR, we treated bmCAF lysates with PNGase F, an enzyme that liberates N-glycans from proteins. PNGase F treatment of bmCAF lysate increased the electrophoretic mobility (and recognition by antibody during IB) of PVR by SDS-PAGE, confirming N-glycosylation of PVR (FIG. 17I). By TB analysis using AAL and anti-PVR, we observed that the pull-down of both fucosylated proteins and sPVR were completely abolished in the L-fuc-supplemented CM but not in the control CM (FIG. 17J; specificity of AAL pull-down was confirmed by L-fuc wash-off). Based on the accumulation of intracellular fuco-PVR detected by L-PLA in bmCAFs treated with BrefA, we tested whether fucosylation is required for the secretion of PVR by LPD comparing CM from control bmCAFs or bmCAFs that were knocked out for SLC35C1 (from FIG. 26C). Consistent with the notion that fucosylation is required for the secretion of PVR, LPD-IB analysis showed that loss of cellular fucosylation decreases PVR secretion (FIG. 17K). To substantiate our findings and further study fucosylated sPVR, we generated bmCAFs that only express sPVR by cloning the endogenous secreted isoform(s) of PVR directly from bmCAFs, which we then ectopically expressed in PVR-knocked-down (shPVR) bmCAFs. Among two soluble isoforms, we observed that isoform 3 (γ) was expressed at high levels in bmCAFs (FIG. 27H). We cloned the sPVRγ isoform from bmCAF cDNA into a lentiviral myc-DDK (also known as myc-FLAG)-tagged plasmid (pLENTI-myc-DDK) that we infected into shPVR bmCAFs. FLAG immunoprecipitation (IP) and IB analysis confirmed that sPVRγ is expressed stably and secreted in shPVR bmCAFs (FIG. 17L). Application of L-PLA to fixed coverslip-grown DDKsPVRγ-expressing bmCAFs confirmed that PVRγ is directly fucosylated and secreted (FIGS. 17M and 27I).

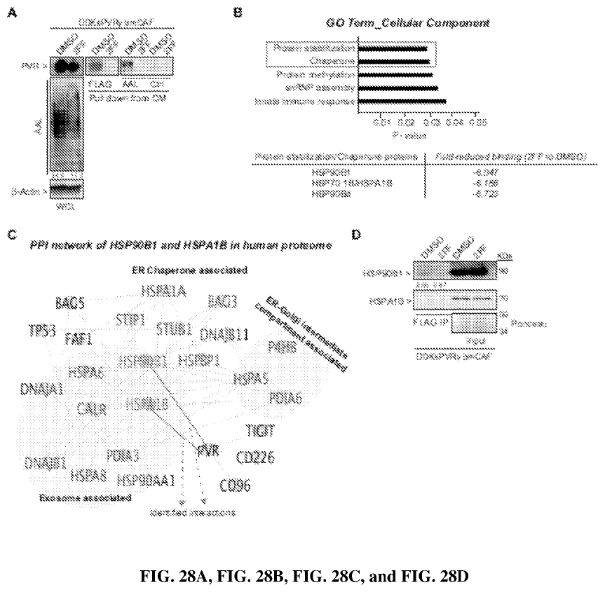

To delineate how fucosylation promotes the secretion of PVR, we assessed the secretion of PVR in DDKsPVRγ-expressing bmCAFs that were pharmacologically modulated for fucosylation by treatment with 2F-peracetyl-fucose (2FF: a FUT inhibitor) or vehicle (dimethyl sulfoxide [DMSO]; control). By IB and LPD-IB analyses, we observed a ~50% loss of AAL signal in the bmCAFs and a complete loss of sfPVR in the CM when cells were treated with 2FF (FIG. 28A). We next performed proteomic profiling of DDKsPVRγ that was FLAG-immunoprecipitated from the lysates of DMSO- or 2FF-treated bmCAFs to identify interactors that might regulate fucosylation-mediated secretion of PVR. This analysis indicated that loss of fucosylation of PVR reduces binding to HSP90B1 and/or HSPA1B proteins (FIG. 28B), which have been reported to promote vesicle-mediated unconventional protein secretion (UPS). Consistently, STRING analysis of previously reported protein-protein interactome networks of HSP90B1/HSPA1B identified protein hubs that are implicated in ER chaperone machinery, exosome, and ER-Golgi intermediate compartment (FIG. 28C). IP-IB analysis validated the direct interaction of HSP90B1 but not HSPA1B with DDKsPVRγ that was lost upon treatment with 2FF (FIG. 28D), strongly suggesting that secretion of fuco-PVR from bmCAFs is mediated via HSP90B1-mediated secretion.

114. Together, these data demonstrate that PVR is highly fucosylated and that fucosylation is required for its HSP90B1-mediated secretion from bmCAFs. As PVR expression correlates with BC staging and poor prognosis, we next sought to delineate whether and how sfPVR from bmCAFs drives BCBM.
(4) PVR fucosylation is driven by FUT1l, a HIF1α-related gene

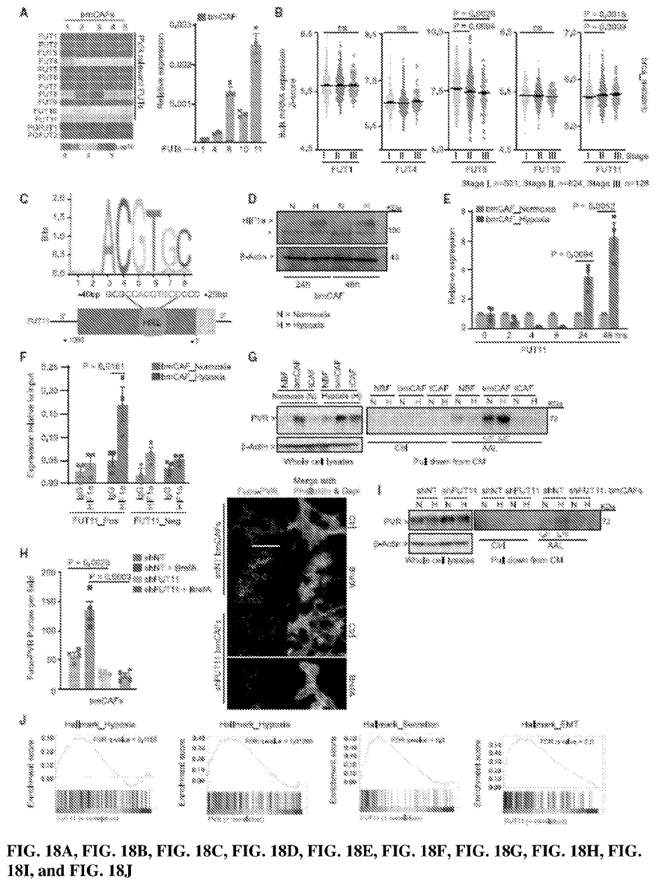

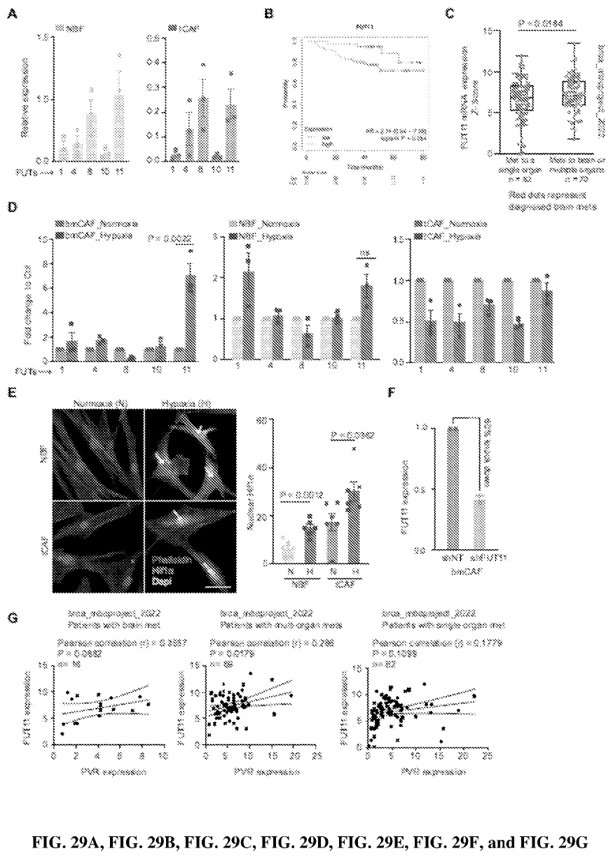

We reasoned that determining how PVR fucosylation is regulated would provide important insight into how and when it is secreted from bmCAFs. One of the key steps in the fucosylation pathway is the addition of L-fuc onto proteins, which is catalyzed by 13 FUTs. To this end, we have established that PVR is N-glycosylated/fucosylated (FIGS. 171 and 17J), which can be catalyzed by 11 of the FUTs. We compared the expression of these FUTs; RNA-seq data revealed that FUT11, FUT10, FUT8, and FUT4 are highly expressed in bmCAFs compared to other FUTs (FIG. 18A, left). RT-qPCR indicated that among the four potential FUTs that can catalyze PVR fucosylation, FUT11 is highly expressed in bmCAFs (FIG. 18A, right). NBFs and tCAFs have a wider range of FUT expression, where there seem to be not a single FUT that is significantly upregulated (FIG. 29A). Analysis of the brca_metabric dataset revealed that FUT11 expression increases significantly with increasing BC stage, whereas FUT8 expression decreases with staging, and changes in FUT1, FUT4, and FUT10 expression are insignificant (FIG. 18B). Kaplan-Meier analysis using KM plotter further revealed that higher FUT11 expression is associated with poorer overall survival in TNBC patients (FIG. 29B). Furthermore, analysis of the brca_mbcproject revealed that FUT11 expression is higher in patients with multi-organ metastasis-who are more likely to develop BM-compared to those with single-organ metastasis (FIG. 29C). These in silico analyses highlight the importance of FUT11 in tumor progression and poor outcomes, although why FUT11 appears selectively upregulated in bmCAFs was unclear.

FUT1 has been described as a hypoxia-related gene and a direct target of HIF1α in multiple cancers including breast cancer. Furthermore, HIF1α can induce proliferation and outgrowth of BC cells within the brain. We reasoned that the chronic hypoxic conditioning experienced by bmCAFs within the brain parenchyma might have resulted in HIF1α-mediated transcriptional regulation of FUT11. Thus, we sought to determine whether FUT11 is transcriptionally regulated by HIF1α. Analysis using Jaspar revealed the presence of a canonical hypoxia response element (HRE) within the FUT11 5′ promoter (FIG. 18C). Culturing bmCAFs under hypoxic conditions stabilized HIF1α protein levels (FIG. 18D) and induced FUT11 (FIG. 18E) expression over 48 h. Chromatin immunoprecipitation (ChIP)-RT-qPCR confirmed hypoxia-stimulated HIF1α binding to the FUT11 5′ promoter at 380-387 bp upstream of the transcription start site (FIG. 18F). Consistent with our HRE analyses in FIG. 18C, we found that no other FUTs were significantly induced by hypoxia in bmCAFs and, further, that a significant upregulation of FUT11 by hypoxia is specific to bmCAFs but not NBF and tCAF (FIG. 29D) regardless of the activation of HIF1α in those cells (FIG. 29E). At the protein level, LPD-IB analysis showed that sfPVR is induced by 2-fold under hypoxia only in bmCAF without a significant change in the total protein level (FIG. 18G). FUT11 knockdown in bmCAFs (FIG. 29F) significantly reduced fuco-PVR cellular puncta, which was not restored by treatment with BrefA (FIG. 18H). Furthermore, LPD-IB analysis showed that secretion of PVR is completely abolished in FUT11-knockdown bmCAFs regardless of their culture in normoxia or hypoxia (FIG. 18I). Using gene set enrichment analysis (GSEA) of the TCGA_BRCA dataset (n=1,085; dbGAP Study Accession: phs000178), we found the following patient breast tumor correlations consistent with our in vitro data: FUT11 and PVR are each positively associated with a hypoxia gene signature; and FUT11 is positively associated with secretion of proteins as well as with EMT (FIG. 18J). Consistent with our findings, analysis of brca_mbcproject revealed that there is a positive trend (r=0.3557) of FUT11 and PVR expression in patients with diagnosed BCBM (FIG. 29G, left) and, along similar lines, FUT11 and PVR expression is positively and significantly correlated in patients with multi-organ metastasis-who are more likely to develop BM-compared to those with single-organ metastasis (FIG. 29G, middle and right).

Together, these data demonstrate that FUT11 is directly transcriptionally upregulated by HIF1α during hypoxia in bmCAFs, which drives the fucosylation and secretion of PVR. The remarkable positive associations of PVR and FUT11 with hypoxia and FUT11 with protein secretion and metastasis signatures in a large breast cancer patient cohort dataset strongly supports potential clinical relevance of our molecular and cellular findings. However, the precise functional and mechanistic contributions of bmCAF sfPVR on BC biology that drive the pathogenesis of BCBM remained unclear.
(5) Phosphoproteomic profiling reveals that sfPVR drives BC cell migration/invasion

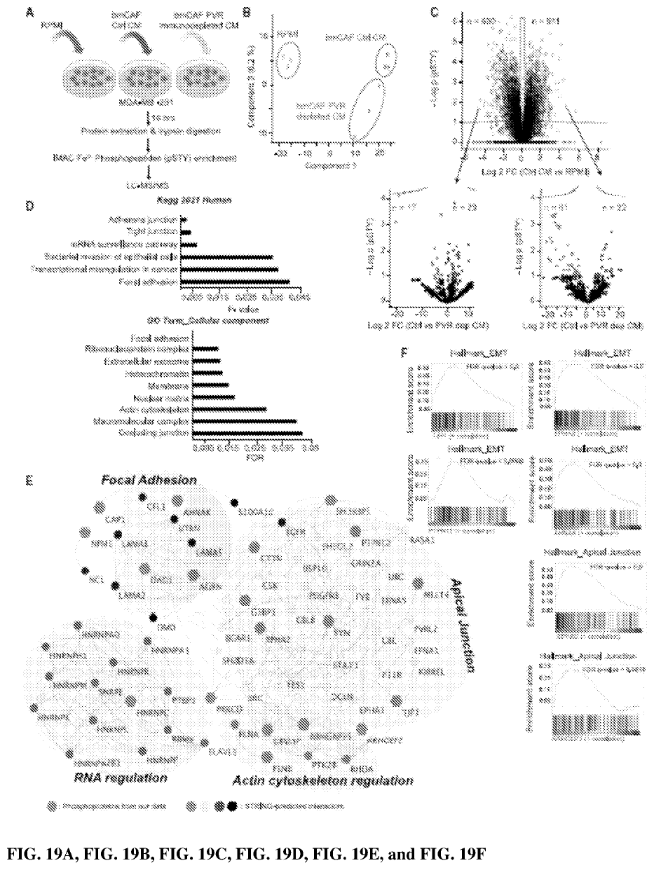

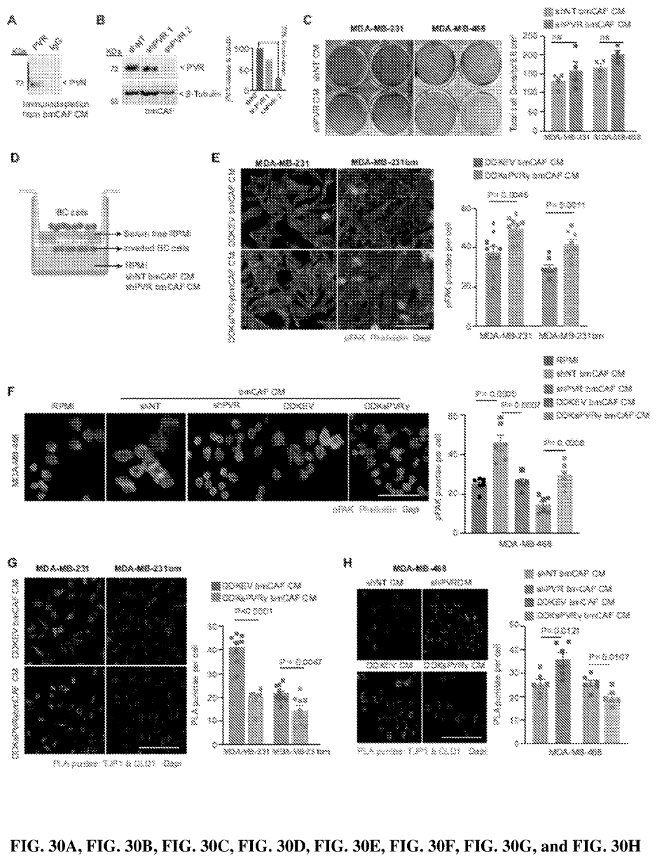

We sought to investigate the functional and mechanistic role(s) of bmCAF sfPVR in promoting BCBM biology and signaling in an unbiased way. To this end, we performed global phosphoproteomic analysis on MDA-MB-231 cells treated with RPMI, or control or PVR-immunodepleted bmCAF CM (FIG. 19A; immunodepletion was confirmed by IB [FIG. 30A]). PCA confirmed high reproducibility between biological triplicates and distinct separation of bmCAF ctrl or PVR-immunodepleted CM-treated groups compared with each other and with the RPMI control (FIG. 19B).

Global phosphoserine/-threonine/-tyrosine (pSTY) analysis identified a total of 9,348 phosphopeptides, 1,541 of which were significantly increased or decreased (≥2 log 2 ratio changed; p≤0.05) in cells treated with control bmCAF CM vs. RPMI (FIG. 19C, upper). We further verified these phosphopeptides as bmCAF-sfPVR-altered by confirmation of reverse phosphorylation status in cells treated with sfPVR-immunodepleted CM. Our comparative analyses ultimately confirmed the identities of 84 BC proteins that were significantly altered in phosphorylation by the presence of sfPVR in bmCAF CM (FIG. 19C, lower).

Kyoto Encyclopedia of Genes and Genomes (KEGG) pathway and gene ontology analyses (5% false discovery rate cut-off) of the 84 proteins, using DAVID Bioinformatics Resources, identified adherens junction, tight junction, focal adhesion, and actin cytoskeleton regulation as pathways significantly altered by sfPVR (FIG. 19D). To explore possible cellular function and signaling changes in a different comparative way, we delineated protein-protein interaction (PPI) networks using STRING analysis. Known interactors with our hits, including but not limited to TJP1, EPHA2, PTPN12, CTTN, AHNAK, ARHGAP35, and MLLT4, identified signaling hubs for apical junction, focal adhesion, and actin cytoskeleton regulation (FIG. 19E). GSEA of the TCGA_BRCA dataset (n=1,085) revealed that, consistent with our findings, expression of TJP1, EPHA2, PTPN12, and AHNAK is positively associated with an EMT signature, and the expression of EPHA2 and ARHGAP35 is positively associated with an apical junction signature (FIG. 19F).

Together, these data demonstrate that bmCAF sfPVR significantly alters actin cytoskeleton remodeling, cell-cell junction, and focal adhesion signaling to induce migration/invasion of BC cells. Furthermore, molecular components and pathways that we delineated as impacted by sfPVR in BC cells correlate strongly with patient expression signatures, emphasizing the clinical implications of our findings in regard to BC migration/invasion.
(6) sfPVR drives BC cell migration/invasion by modulating the adherens and tight junctions

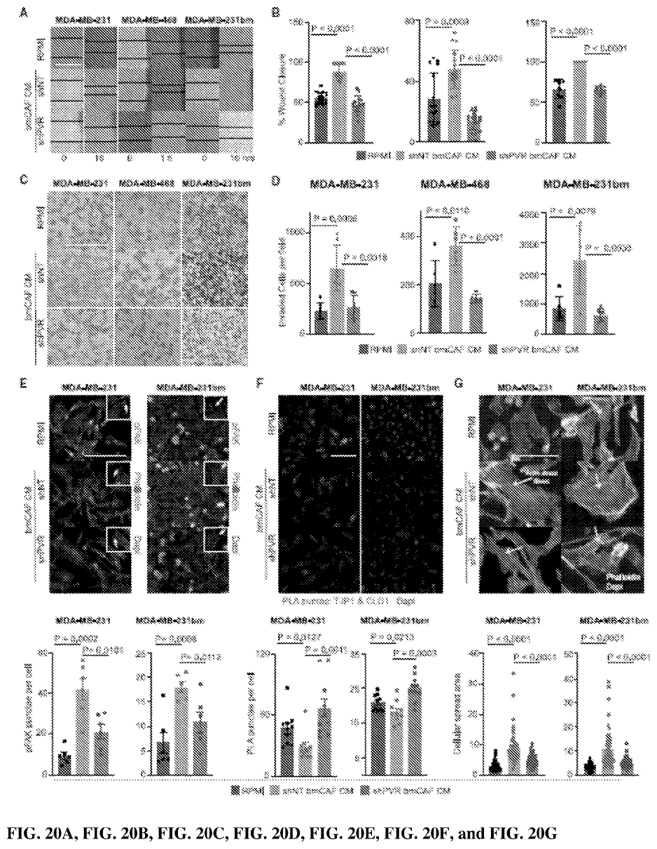

To functionally validate our phosphoproteomic finding that sfPVR potentially plays an important role in driving BC cell migration/invasion, we assessed the migration and invasion of BC cells treated with CM from control (shNT) or PVR-knocked-down (shPVR) bmCAFs (~70% knockdown of PVR at the protein level; FIG. 30B). Wound-healing (2D migration) assay revealed that compared to RPMI, CM from shNT bmCAFs increased the wound closure of MDA-MB-231, MDA-MB-468, and brain-homing MDA-MB-231 (MDA-MB-231bm) cells, whereas shPVR CM did not (FIGS. 20A and 20B). To rule out the possibility that sfPVR could impact the proliferation of BC cells, we performed a colony-formation assay which confirmed that sfPVR did not perturb proliferation of BC cells (FIG. 30C). Likewise, a Matrigel invasion assay in which shNT or shPVR CM was used as chemoattractant (FIG. 30D) revealed that shNT CM significantly induced the invasion of BC cells through the Matrigel plug, whereas shPVR CM did not (FIGS. 20C and 20D).

We next sought to verify whether these motility/invasion changes were mediated because of altered adherens junctions, as suggested by our phosphoproteomic analyses. One of the key adherens junction proteins, focal adhesion kinase (FAK), serves as a unique regulator of focal adhesion assembly-disassembly and cytoskeletal remodeling, processes that are fundamental for efficient directional cell movement. IF staining analyses revealed that compared to RPMI, CM from shNT bmCAFs increased pFAK puncta at the leading edge of BC cells, whereas shPVR CM did not (FIGS. 20E and 30F). Consistently, CM from DDKsPVRγ bmCAFs also increased pFAK puncta more than CM from DDKEV bmCAFs (FIGS. 30E and 30F). These observations are consistent with previous reports that PVR enhances cell dispersal upon activation of Src/FAK signaling.

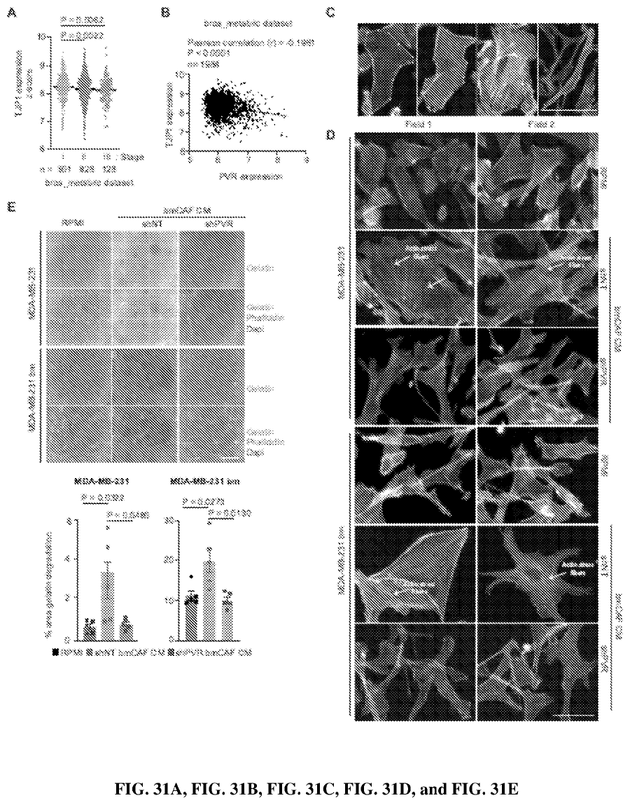

Our phosphoproteomics data suggested that in addition to adherens junction, sfPVR regulates tight junction (TJ) dynamics by modulating tight junction protein 1 (TJP1). TJP proteins are involved in maintaining the integrity and architecture of interacting cells via actin cytoskeletal interactions; disruption of TJs during tumorigenesis enhances invasiveness of BC cells. To evaluate whether sfPVR alters TJs in BC cells, we performed PLA on TJP1 and its binding partner claudin 1 (CLD1). PLA analyses revealed that compared to RPMI, CM from shNT bmCAFs reduced the number of TJP1- and CLD1-containing complexes per BC cell, whereas shPVR CM did not (FIG. 20F). Consistently, CM from DDKsPVRγ bmCAFs also decreased the number of TJP1:CLD1 complexes per BC cell compared to CM from DDKEV bmCAFs (FIGS. 30G and 30H). Analysis of the brca_metabric dataset revealed that TJP1 expression is reduced in stage II and III compared to stage I patients (FIG. 31A), and further that PVR expression is negatively correlated with TJP1 expression (FIG. 31B), consistent with our in vitro data showing that sfPVR perturbs TJP1/TJs to promote invasion of BC cells.

As the driving force for cell migration and invasion is mediated by actin cytoskeletal polymerization and rearrangement, including cell spreading and stress fiber formation, we next evaluated the effects of bmCAF sfPVR on cell spreading and actin stress fiber formation in BC cells. Phalloidin staining revealed that compared to RPMI, CM from shNT bmCAFs induced cell spreading and increased actin stress fiber formation in BC cells, whereas shPVR CM did not (FIGS. 20G, 31C, and 31D). As invasion of cancer cells relies on the degradation of the tissue matrix by metalloproteinase, we assessed whether sfPVR could enhance the matrix degradative activity of BC cells. A gelatin-degradation assay revealed that compared to RPMI, CM from shNT bmCAFs significantly enhanced the gelatin-degradation capacity of BC cells, whereas shPVR CM did not (FIG. 31E).

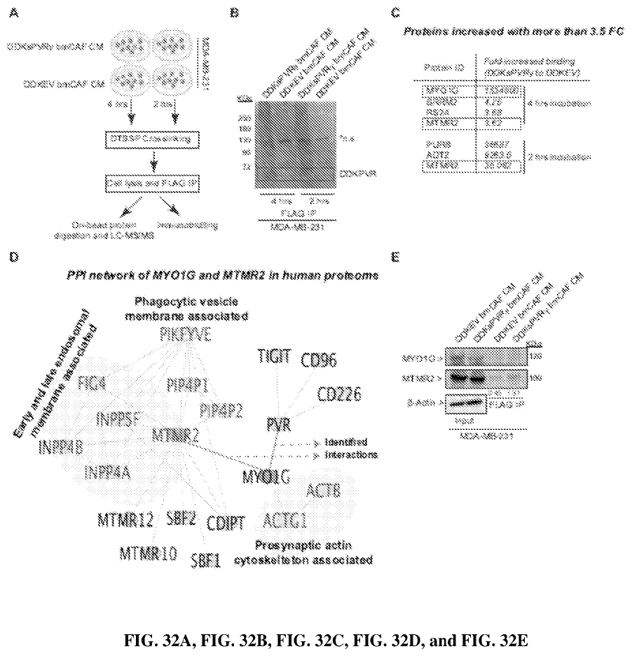

To delineate the potential cell surface binding partner(s) of sfPVR on BC cells, we cultured MDA-MB-231 cells with CM from DDKsPVRγ bmCAFs or DDKEV bmCAFs and performed chemical crosslinking at 2 h and 4 h of CM incubation time using 3,3′-dithiobis(sulfosuccinimidylpropionate) (DTSSP). FLAG immunoprecipitation of DDKsPVRγ and LC-MS/MS analysis of the bound MDA-MB-231 proteins (FIGS. 32A and 32B) identified MTMR2 and MYO1G as top interactors of DDKsPVRγ (FIG. 32C). PPI network analysis of MTMR2/MYO1G highlighted protein hubs that are implicated in early and late endosomal-membrane-associated, phagocytic-vesicle-membrane-associated, and prosynaptic-actin-cytoskeleton-associated signaling (FIG. 32D). IP-IB analysis confirmed the direct interaction of MTMR2 with DDKsPVRγ (FIG. 32E). Myotubularin-related protein 2 (MTMR2) is a 3-phosphatase specific for phosphoinositides PI(3)P and PI(3,5)P₂; PI(3)P plays a role in vesicular trafficking, endocytosis, and membrane transport.

That we did not identify a canonical cell surface “receptor” for PVR on the surface of BC cells but rather identified a mediator of endocytosis (MTMR2) is consistent with our earlier finding that fuco-PVR is secreted via HSP90B1- and vesicle-mediated UPS (FIG. 28) and is taken up into BC cells via endocytosis to regulate adherens junction, TJ, and actin cytoskeleton dynamics to drive BC cell migration/invasion.
(7) sfPVR promotes invasive spread of BC cells within the brain

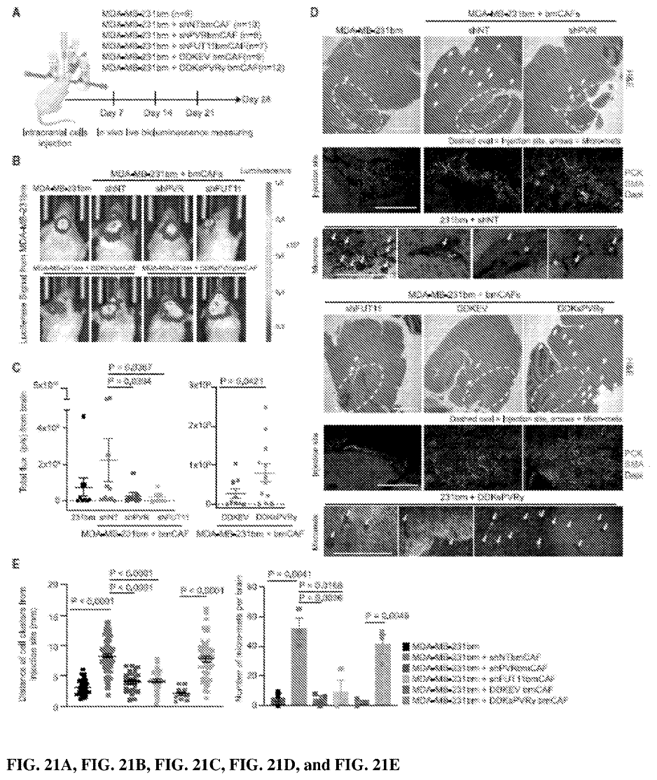

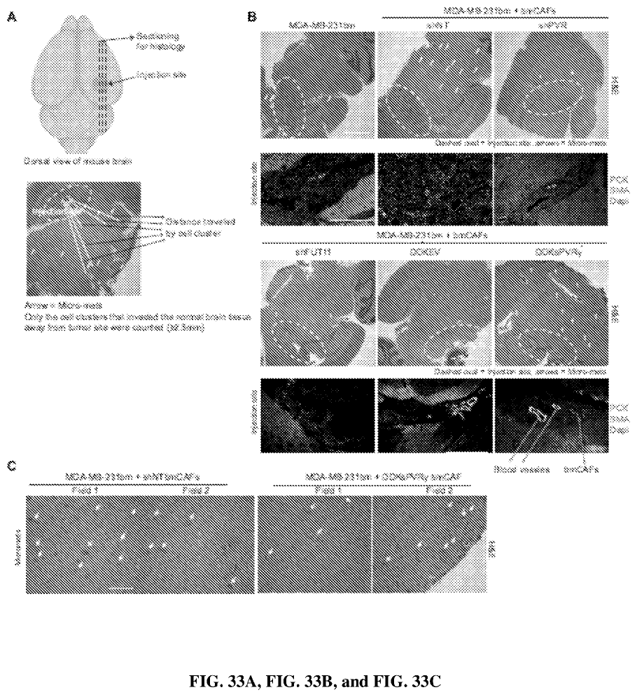

Our in vitro findings supported the possibility that sfPVR from bmCAFs would support the colonization/invasion of BC cells within the brain parenchyma to develop BCBM. To test this notion, we intracranially implanted immunodeficient female NSG mice with brain-homing, luciferase-expressing MDA-MB-231bm BC cells alone or co-injected with control, PVR-knocked-down, PVR-overexpressing, or FUT11-knocked-down bmCAFs, and tumor growth was monitored over time (FIG. 21A). Bioluminescence imaging revealed that the co-injection of control bmCAFs increased the intracranial growth of the MDA-MB-231bm tumors compared with MDA-MB-231bm cells alone. In contrast, the co-injection of either PVR- or FUT11-knocked-down bmCAFs did not increase tumor growth (FIGS. 21B and 21C, left). Compared with control bmCAFs, co-injection with PVR-overexpressing bmCAFs significantly increased tumor growth (FIG. 21B, and 21C, right), demonstrating that sfPVR is a predominant driver of breast tumor establishment/spread in the brain. Immunohistochemical staining of brain sections and quantification of the distance and number of BC cell clusters from the injection site confirmed significant spread of MDA-MB-231bm cells and the formation of micrometastases within the brain parenchyma induced by control and, further, by PVR-overexpressing bmCAFs (FIGS. 21D, 21E, and 33A-33C). Furthermore, we were able to immunofluorescently detect the presence of both MDA-MB-231bm and bmCAFs at the tumor endpoint (FIG. 21D), confirming the persistence of bmCAFs during the experiment and further highlighting their role in BCBM invasiveness. Our in vivo findings verify our in vitro observations, demonstrating that sfPVR from bmCAFs promotes BCBM development by driving the invasive spread of BC cells in the brain.
(8) Fucosylated PVR is elevated in bmCAFs from BCBM patients

Given the potent enhancement of BCBM development by sfPVR from bmCAFs in our animal models, we sought to investigate whether total and fucosylated PVR was also increased in bmCAFs in BCBM patient tissue specimens. To this end, we immunofluorescently stained BCBM tissue biopsies from ten patients for total/fuco-PVR, BC cells, and fibroblasts (FIGS. 22A-22F). Single-cell segmented imaging analysis revealed that bmCAFs had higher total average PVR levels per cell compared to BC cells (FIGS. 22A-22C). Importantly, lectin-mediated proximity ligation (FIG. 27D) followed by single-cell segmentation revealed that fuco-PVR levels are also significantly higher in bmCAFs than in BC cells (FIGS. 22D-22F). Notably, almost all bmCAFs within the brain immunostained positively for fuco-PVR in nine of the ten patients (FIG. 22G) and, moreover, PVR was predominantly fucosylated in six of the ten patients (FIG. 22H). Notably, BCBM tumors in younger patients (n=6) contained strikingly more bmCAFs that exhibited higher fuco-PVR levels compared to tumors in older patients (n=4) (FIG. 22I, left and right). This intriguing observation of potentially more tumorigenic bmCAFs in the younger BCBM patients is consistent with reports that younger patients are more likely to develop brain metastases. Together, our findings confirm that total and fucosylated PVR are upregulated in bmCAFs in the tumors of BCBM patients, supporting their pathogenic roles that we have delineated in our in vitro and pre-clinical models.
b) Discussion

CAFs have been well established to play functional roles that significantly influence the development and metastatic progression of multiple cancer types, including BC. However, the presence and impact of CAFs in BCBM development has been controversial and poorly understood. Nonetheless, recent studies have identified CAFs within BCBM patient biopsies and have reported that these bmCAFs can enhance BCBM by enhancing the migration of patient-derived cancer cells, disrupting the BBB, or paracrine signaling via PDGFB-to-PDGFRβ. Although these studies have begun to provide insight into the development of BCBM, our in-depth understanding of bmCAF-mediated molecular mechanisms is limited.

Here, we report a new functional relationship between fucosylation, bmCAFs, and the development of BCBM. We discovered a new hypoxia- and fucosylation-regulated mechanism by which bmCAFs secrete fucosylated PVR that promotes the invasion and metastatic spread of BC cells within the brain. Our study provides important mechanistic insights into BCBM pathogenesis, highlighting, for the first time, bmCAF-secreted/fucosylated PVR and FUT11 as potential new therapeutic targets with implications as a potential biomarker for BCBM.

Among the four different PVR isoforms, only tPVR has been extensively studied; roles of sPVR in cancer are unknown. tPVR is overexpressed in multiple cancers including glioblastoma and can play key roles in tumor cell migration/invasion by altering αvβ3 integrin- and Src/FAK-mediated cellular adhesion and motility. tPVR expressed on tumor cells can also interact with DNAM-1 and TIGIT and attenuate anti-tumor immunity, representing an emerging immune checkpoint mechanism. In humans, elevated sPVR serum concentrations have been reported across a broad spectrum of cancer patients (including lung, breast, ovarian, and colorectal cancers) compared to healthy individuals. sPVR expression has also been reported to be increased in later-stage cancers (stages 3 and 4) than in early cancers (stages 1 and 2), consistent with our finding that bmCAFs express higher sPVR levels compared to tCAFs or NBFs (FIG. 17). However, the functional role(s) of sPVR and its regulation in tumor cells have until now been largely unstudied. As the extracellular domains of tPVR are conserved in sPVR, it is believed that sPVR may have functions similar to those of tPVR. Indeed, similar to tPVR, sPVR can inhibit DNAM-1-mediated anti-tumor activity of NK cells.

Our study provides significant insight into the post-translational modification and regulation of pathological sPVR function in BC: we delineate the novel fucosylation-regulated secretion and function of sPVR in modulating cell-cell contacts and focal adhesion complexes and driving BC invasiveness. As PVR is upregulated in multiple cancer types including lung, ovarian, and colorectal, this mechanism may be conserved among a wide range of cancer types. Importantly, as it is a secreted protein, we anticipate that sfPVR would reach cerebrospinal circulation, and thus our study raises the possibility for sfPVR to be a new predictive biomarker for BCBM that can be assessed by liquid biopsy of cerebrospinal fluid. BCBM currently lacks effective early diagnostic screening strategies and is generally only screened for or diagnosed when BC patients present with clinical neurological signs and symptoms-well after BM cells have spread and invaded into the brain parenchyma.

Thus, the further study of the utility of sfPVR as a potentially early and relatively easily accessed biomarker is expected to significantly advance our ability to diagnose BCBM.

The crucial roles played by fucosylation in regulating cellular signaling are an emerging area of study, particularly in regard to its deregulation and pathological contributions to cancer development and progression. One common cause of altered fucosylation in cancer is the aberrant expression of FUTs. More than 85% of secreted human proteome are glycoproteins, which are often fucosylated. Since fucosylation machinery is generally localized within the secretory pathway, fucosylation has been thought to predominately regulate transmembrane and secreted proteins. Our discovery that HIF1α-upregulated FUT11 fucosylates PVR and triggers its secretion by bmCAFs (FIGS. 17 and 18) highlights how aberrant fucosylation can result in the pathological secretion of proteins that promote tumorigenesis—in this case, specifically by enhancing invasive capacity. Moreover, that HIF1α mediates this process emphasizes the context-specific pathological contribution of altered fucosylation: chronic hypoxia within the brain parenchyma likely underlies the unique FUT11 expression and fucosylation of PVR in bmCAFs that is not readily observed in CAFs from other sites in the body. Our findings highlight and help to explain hypoxia-induced, stromal contributions to BCBM biology, reinforcing the importance of hypoxia and HIF1α in mediating the outgrowth and therapeutic resistance of BC and other tumor types in the brain. Furthermore, our data support the targeting of sfPVR by neutralizing antibody and/or targeting the cell-cell adhesion interactions in BC cells that are modulated by sfPVR, as well as further assessment of its utility as a cerebrospinal fluid biopsy biomarker. Our elucidation of the mechanistic determinants is expected to advance our understanding of CAF-driven BCBM pathogenesis and to inform efforts in implementing such fucosylated soluble proteins as novel biomarkers.
c) Materials and Methods
(1) Cell lines

MDA-MB-231, MDA-MB-468, and HEK293T cells were purchased from American Type Culture Collection (ATCC). MDA-MB-231bm cells were obtained from the Joan Massague lab at Sloan Kettering Institute. MDA-MB-231 and MDA-MB-468 cells were cultured in RPMI 1640 (Corning) containing 10% fetal bovine serum (FBS; PEAK Serum). MDA-MB-231bm and HEK293T cells were cultured in Dulbecco's Modified Eagle's Medium (DMEM) (Cytiva), high glucose containing 10% FBS. The identities of all cell lines in the Lau laboratory are verified annually by short tandem repeat-based authentication ‘Cell Check’ services provided through IDEXX Bioresearch.

Normal human astrocytes and fibroblasts were gifts from the Marusyk lab at Moffitt Cancer Center & Research Institute, Tampa, FL. Normal human astrocytes were purchased from Lonza and Cell Application. Tumor CAFs and brain met CAFs were isolated from BC patient samples and expanded for 3-10 passages prior to the experiment. All human tissue was collected using protocols approved by the Dana-Farber Harvard Cancer Center (DF/HCC) Institutional Review Board. Fibroblast culture and all of the in vitro experiments were performed in media containing human mammary epithelial cell growth medium (Sigma Aldrich) and DMEM/F-12 (50:50, Corning) that contains 5% FBS and 5 mg insulin (Sigma Aldrich). All cells were incubated at 37° C. at 5% CO₂ in a humidifier incubator.
(2) Animal studies

All mouse experiments were performed in accordance with an Institutional Animal Care and Use Committee protocol (IACUC protocol, #IS00010075) approved by the University of South Florida. Four-to-six-weeks old female NSG mice were used in this study, which were obtained from Lau laboratory breeder colonies of Moffitt Cancer Center vivarium. Mice were randomly divided into groups and at least 10 mice per cohort was used. 1×10⁴ luciferase tagged MDA-MB-231bm cells alone or mixed with bmCAFs at 1:1 ratio was implanted intracranially by performing stereotactic surgery. Tumor growth in the brain was measured by monitoring luciferase signal every week by using IVIS. At the endpoint, mice were euthanized, brains were resected and fixed in formalin. Tissue embedding in paraffin, sectioning, and hematoxylin and eosin (H & E) staining were performed by Tissue Core at Moffitt Cancer Center. H & E sections and immunofluorescent staining (performed as described above) were observed, and images captured using Keyence BZ-X710 microscope.
(3) Cloning and mutagenesis
   (a) Cloning of sPVR from bmCAF

We used the In-Fusion HD cloning kit from TakaRa Bio (Cat #639650) to clone sPVRγ into the pLenti-C-Myc-DDK-IRES-Puro expression vector (Ori-Gene Technologies). Briefly, RNA from bmCAF was extracted and converted into cDNA (High Capacity cDNA Reverse Transcription kit, Applied Biosystem, Cat #4368814). sPVRγ (NCBI Ref Seq; CCDS46105.1) was PCR-amplified from cDNA using In-Fusion primers, and the In-Fusion cloning reaction was carried out to clone PVR into Not1-linearized pLENTI-myc-DDK-IRES-Puro plasmid. Infusion primers were designed using SnapGene in such a way that they generate PCR products containing ends that are homologous to those of the vector. Lentiviral particles were generated using HEK293T cells transfected with control (pLenti-C-Myc-DDK-IRES-Puro; a.k.a., DDKEV) or pLenti-C-Myc-DDK-IRES-Puro-PVR (DDKsPVRγ) lentiviral vectors along with VSVG and Δ8.9 packaging vectors. bmCAFs were subsequently infected with the prepared lentivirus, followed by antibiotic selection (1.0 ug/ml puromycin (InvivoGen)).
(b) shRNA knockdown of bmCAFs

Using the same lentiviral production and infection methods described above, shRNA-encoding plasmids (pLKO.1 lentiviral vector) (MISSION shRNA, Sigma-Aldrich) were used to stably knockdown human PVR and FUT11 genes in bmCAFs. Three shRNAs for each gene were used for lentiviral infection. After qRT-PCR validation, the construct with the most knockdown efficacy was used for functional assays. Lentiviral sequences for shPVR and shFUT11 are provided in Table 1.
(c) Generation of luciferase-expressing MDA-MB-231bm cells

The Cignal Lenti luciferase system (CLS-PCL-8) from Qiagen was used to generate luciferase-expressing MDA-MB-231bm cells. Briefly, 50,000 cells were plated into a 12-well plate, infected with viral particles, and the plate was centrifuged for 2 h at 400 g at RT. The plate was cultured for 24 h, followed by media change and recovery for 72 h. The cells were then selected using 1.0 ug/ml Puromycin, and finally luciferase signal was measured using D-Luciferin substrate in a Promega GloMax Luminometer.
(4) Gene expression profiling of fibroblasts

We acquired raw read counts and sample meta data from GEO entry GSE80333; accessed 2023-08-02) and loaded into RStudio (version 2023.06.1; using R 4.0.4). We used the R edgeR function filterByExpro with default arguments to remove genes with low counts, resulting in 15,464 genes for further analysis. Trimmed mean of M values (TMM) normalization was next applied, followed by data transformation using the R package voom. Differentially expressed genes between bmCAF and tCAF were determined with the R package limma (25605792). Genes with adjusted p value <0.05 were considered significant.
(5) Fibroblasts conditioned media (CM) generation

To generate CM, we plated and cultured fibroblasts in a 10-cm² dish to ~70-80% confluence. The fibroblast media was then changed to serum free (sf)-RPMI and further incubated for 48 h. The sf-RPMI was then collected, clarified by centrifugation (5 min at 1,000 g), aliquoted, and used immediately or stored frozen at −80° C. until needed.
(6) Lectin Pulldown from Fibroblast Conditioned Media (CM)

Conditioned media (CM) from NBFs, tCAFs, and bmCAFs were collected on ice, pelleted to remove any debris, and the supernatants were normalized to protein concentration of the whole cell lysates with the lowest concentration. Control beads and AAL- or UEA-1 lectin-conjugated agarose beads (Vector Laboratories) were pre-blocked for 2 h in blocking buffer (2% IgG-Free BSA (Jackson ImmunoResearch Laboratories)) on a rotator at 4° C. Cleared CM was incubated with 50 μL of pre-blocked beads (beads were spun out of blocking solution and resuspended in dilution buffer: 0% Triton X-100, 20 mM Tris-HCL, pH 7.4, 150 mM NaCl and ddH₂O with protease and phosphatase inhibitors) and rotated overnight at 4° C. The next day, the beads were washed twice with PBST (PBS supplemented with 0.1% Tween 20), subjected to (10%) SDS-PAGE, and IB analysis using the indicated antibodies. We established the specificity of lectin binding AAL to L-fuc by performing L-fuc wash-off experiments, where the AAL-conjugated beads were first pre-incubated or not with 500 mM L-fuc, followed by incubation with bmCAF CM, followed by either standard wash or 500 mM L-fuc-supplemented wash.
(7) Mass spectrometric analyses
   (a) Profiling fucosylated proteins of bmCAFs secretome

Conditioned media (CM) from bmCAFs and NBFs were pulled down using AAL lectin-conjugated agarose beads as described above. Control and AAL agarose beads were washed with PBS and subjected to in-gel trypsin digestion. Bead-bound proteins were denatured at 95° C. for 5 min and then loaded onto an SDS gel. After 20 min electrophoresis, the gel was rinsed with water and stained with Instant Blue solution (Abcam) for 30 min. Protein-containing gel segments were excised and cut into cubes, followed by de-staining with 50 mM Ambic/50% methanol, reduction with 25 mM Ambic/2 mM TCEP, alkylation with 25 mM Ambic/20 mM IAA, and digested overnight at 37° C. with an enzyme-to-protein ratio of trypsin at 1:20 (Worthington). Peptides were extracted from the gel by incubating with 50% acetonitrile/0.1% TFA for 20 min at RT. The resulting peptide solutions were purified by Ziptip procedure (Millipore). The eluted peptides were dried in a speedvac and suspended in 15 μL loading buffer (2% CAN and 0.1% TFA). LC-MS/MS and data analyses were performed as described below.
(b) Phosphoproteomic profiling of BC cells

MDA-MB-231 cells were treated with RPMI, or ctrl- or PVR-immunodepleted bmCAF CM for 16 h. After 16 h, cells were collected on ice, lysed with urea lysis buffer (20 mM HEPES pH 8.0, 9 M urea, 1 mM sodium orthovanadate, 2.5 mM sodium pyrophosphate, 1 mM β-glycerophosphate), and protein concentrations were determined by DC Protein Assay (Bio-Rad). One milligram of each lysate was reduced with 4.5 mM dithiothreitol for 30 min at 60° C., alkylated with 10 mM iodoacetamide at RT in the dark for 20 min and digested overnight at 37° C. with an enzyme-to-protein ratio of trypsin at 1:20 (Worthington). Resulting peptides were desalted using a reversed-phase Sep-Pak C₁₈ cartridge (Waters) and lyophilized for 48 h. Lyophilized peptides were enriched for global phosphopeptides (pSTY) using IMAC Fe-NTA magnetic beads (Cell Signaling Technology), as described by manufacture protocol on a KingFisher Flex Purification System (Thermo Fisher Scientific), followed by SpeedVac concentration. Peptides were then resuspended in loading buffer (5% CAN and 0.1% TFA) before auto-sampling and LC-MS/MS as described below.
(c) Liquid chromatography-mass spectrometry

A nanoflow ultra-high-performance liquid chromatography (RSLC, Dionex) connected to a Q-Exactive Plus mass spectrometer (ThermoFisher Scientific) was used for tandem mass spectrometry peptide sequencing. The LC-MS/MS was performed by the Proteomics Core at Moffitt Cancer Center. Briefly, peptide mixtures were first loaded onto a pre-column (2 cm×100 μm ID packed with C18 reversed-phase resin, 5 m, 100 Å) and washed for 8 min with aqueous solvent A. The trapped peptides were eluted onto the analytical column (C18, 75 μm ID×25 cm, 2 μm, 100 Å, Dionex, Sunnyvale, CA). The 120-min gradient was programmed as follows: 95% solvent A for 8 min, solvent B (90% ACN+0.1% FA) from 5% to 38.5% in 90 min, then solvent B from 50% to 90% in 7 min and held at 90% for 5 min, followed by solvent B from 90% to 5% in 1 min and re-equilibrate for 10 min. The flow rate on the analytical column was 300 nL/min. Twenty tandem mass spectra were collected using data-dependent acquisition (DDA) following each survey scan. The resolution settings were 60,000 and 17,500 for MS1 and MS/MS, respectively. The isolation window was 2.0^{Th} with an offset of 0.5.

The data were processed and analyzed using MaxQuant software (version 1.5.2.8). The fragment mass tolerance was set to 20 ppm. Peptides with a minimum of 7 amino acids and a maximum of 2 missed cleavages were considered. Methionine oxidation, N-terminal acetylation, and serine/threonine/tyrosine phosphorylation were selected as variable modifications. Carbamidomethylation of cysteine was used as the fixed modification. The false discovery rate (FDR) was applied at 0.05. MaxQuant data was further normalized with IRON (Iterative Rank-Order Normalization) within each dataset. Pathway enrichment analyses were performed on DAVID (Functional Annotation Tool).
(8) Cell viability (XTT) assay

Cell viability was measured by using XTT reagent from Thermo Fisher (Cat #X6493). BC cells were plated (3,000 cells/well) in a 96-well plate, treated with RPMI, NBF-, tCAF-, or bmCAF-derived CM that was depleted or not of fucosylated proteins and incubated for 72 h at 37° C. After 72 h, XTT (2,3-Bis-(2-Methoxy-4-Nitro-5-Sulfophenyl)-2H-Tetrazolium-5-Carboxanilide) was added at 2 mg/ml followed by phenazine methosulfate (3 μm) to a final volume of 125 μL. Cells were incubated for an additional 4 h at 37° C. prior to measuring the absorption at 490 nm.
(9) Immunoblotting

Cells treated as indicated were washed with ice-cold PBS and lysed on ice in standard RIPA buffer (25 mM Tris-HCl, pH7.6, 150 mM NaCl, 5 mM EDTA, 1% NP-40 or 1% Triton X-100, 1% sodium deoxycholate, 0.1% SDS in diH2O with protease and phosphatase inhibitors). Protein lysates were sonicated, pelleted, and the resulting lysates were normalized by protein concentration using the DC assay (Bio-Rad Laboratories). Equal amounts of heat-denatured proteins were loaded onto 10% SDS-PAGE gel and transferred onto a polyvinylidene fluoride (PVDF) membrane (Bio-Rad). Membranes were blocked with 5% non-fat milk or Carbo-Free blocking solution (Vector Laboratories) in TBST for 30 min, followed by incubation with primary antibodies overnight at 4° C. The next day, membranes were washed and incubated with horseradish peroxidase (HRP)- or infrared dye (IRDye)-conjugated secondary antibodies for 1 h at RT. After 3-5 washes in TBST, membranes were imaged using Odyssey Fc Imaging System (LI-COR Biosciences).
(10) PNGase F digestion

PNGase F digestion was performed using the amidase from New England BioLabs, Cat #P0704S. Briefly, 15 μg of protein lysate was combined with 1 μL of glycoprotein denaturing buffer (10×) and H₂O in a total reaction volume of 10 μL. Glycoproteins were denatured by heating reaction at 100° C. for 10 mins, denatured glycoprotein was chilled on ice, and centrifuged for 10 s. Each PNGaseF reaction was performed in a final volume of total of 20 μL (by adding 2 μL of GlycoBuffer 3, 2 μL of 10% NP40, 6 μL H₂O, and 2 μL PNGaseF). The reaction was incubated at 37° C. for 1 h, and PVR digestion was analyzed by immunoblotting.
(11) Quantitative PCR with reverse transcription

RNA from the indicated cells was extracted using the GeneElute Mammalian Total RNA extraction system (MilliporeSigma) and reverse transcribed using the High-Capacity cDNA Reverse Transcription Kit (Thermo Fisher Scientific). RT-qPCR was performed using FastStart Universal SYBR Green Master Mix (Rox) (Roche Diagnostics) using CFX Manager version 3.1 on a Bio-Rad CFX96 Real-Time system (Bio-Rad Laboratories). RT-qPCR cycles were as follows: 95° C. for 10 min, 35 cycles of 95° C. for 15 s, 55° C. for 60 s and 72° C. for 30 s. Gene expression was normalized to histone H3A expression. Primers for RT-qPCR were generated using NCBI primer BLAST software (National Center for Biotechnology Information). Oligonucleotide sequences are provided in Table 1.
(12) Chromatin immunoprecipitation (ChIP)-qRT

HIF1α Chip-qRT protocol was modified from the published Chip-qRT protocol. Briefly, cells were crosslinked using 1% formaldehyde for 15 min at RT and then quenched with 5 M glycine. Cells were lysed and sonicated to obtain 500-bp chromatin fragments (as assessed by DNA gel). Five hundred micrograms of chromatin were immunoprecipitated overnight at 4° C. with 5 μg of HIF1α antibody (Active Motif Cat #39665). The next day, protein/DNA complexes were pulled down using 30 μL of pre-blocked protein A/G beads for 6 h. Beads were washed for 4 times, the crosslinking of protein/DNA complexes was reversed, and the DNA was then purified using spin columns and subjected to standard qPCR analysis.
(13) Hypoxia

Biospherix Hypoxia Chamber was used as the oxygen content controller to allow the creation of a full range of oxygen content regulation from 0.1% to 99.9%, as well as CO₂ control from 0.1% to 20.0%. To create hypoxia, NBFs, tCAFs, or bmCAFs were cultured in this chamber for 48 h maintaining an O₂ concentration of 0.5%, at 5% CO₂ and 37° C. A twin cell culture was placed in the normoxia as a control. After the desired incubation period, growth media was removed from cells, cells were rinsed with cold PBS, and immediately collected on ice and processed for RNA extraction or immunoblotting.
(14) Wound healing migration assay

MDA-MB-231, MDA-MB-468, or MDA-MB-231bm cells were grown in a 12-well plate to 100% confluency, washed once with PBS, and scratches were made into the cell layer using a sterile 20 μL pipette tip. Dead cells and debris were washed off with PBS. The cells were then treated with RPMI, shNT- or shPVR bmCAFs CM containing 5% FBS. The same area was photographed directly after scratching and at 16 h. The width of the initial wound and of the wound after 16 h was measured by ImageJ software. The % wound closure was calculated as the 16 h wound width divided by the initial wound width.
(15) Matrigel invasion assay

We performed Matrigel invasion assays using Corning BioCoat Matrigel Invasion Chamber (Cat #354480). Briefly, the Matrigel transwells were removed from −20° C. storage, allowed to come to RT, and warm sf-RPMI media was added to the interior of the inserts and bottom of wells. Matrigel was allowed to rehydrate for 2 h in humidified tissue culture incubator. After rehydration, the medium was carefully removed without disturbing the layer of Matrigel matrix on the membrane. BC cells were plated on a 6-well plate and were treated with RPMI, shNT- or shPVR bmCAFs CM for 16 h. The next day, the cells were resuspended using enzyme-free detachment solution, counted using cell counter, and 100,000 cells/well (in sf-RPMI) were plated into the inserts of the rehydrated Matrigel invasion chambers. RPMI, shNT- or shPVR bmCAFs CM was placed in the bottom chamber as a chemoattractant. After 16 h, the non-invaded cells were removed from the upper chamber surface by cotton swabs, and the invaded cells on the lower surface were stained by crystal violet and counted. The invasion was calculated as the percentage (%) of the number of penetrated cells divided by the total number of cells plated.
(16) Gelatin degradation assay

BC cells were cultured in 6-well plates and treated with RPMI, shNT- or shPVR bmCAF CM for 16 h. The next day, the cells were resuspended and seeded into 8-well chamber slides coated with FITC-conjugated gelatin (Invitrogen) (3×10⁴ cells/well). After 16 h of culture with the indicated treatments, the cells were fixed, permeabilized, and blocked as described below in the IF staining section. The cells were subsequently stained with AlexaFluor 594-Phalloidin (ThermoFisher Scientific). The slides were mounted and imaged using a Keyence BZ-X710 fluorescent microscope. Quantification of the areas devoid of FITC (i.e., degraded gelatin areas) was performed using Fiji software (NIH).
(17) DTSSP crosslinking

DTSSP crosslinking was performed using the reagents from Thermo Fisher Scientific (Cat #21578). Briefly, MDA-MB-231 cells on a 20 cm plate were treated with CM from DDKEV or DDKsPVRγ bmCAFs for 2 h or 4 h and crosslinking was performed at RT using 1.5 mM DTSSP for 30 min. After 30 min, 10 mM Tris, pH 7.5 was used as a stop solution for 15 min at RT. Finally, CM was removed from the plate, cells were washed with PBS, scraped from the plate, lysed with IP buffer, and FLAG pulldown was performed. FLAG beads were washed with TBS and proceed for Immunoblotting or on-bead digestion for LC-MS/MS.
(18) Immunofluorescent staining
   (a) Immunofluorescent cell staining

Cells were grown on German glass coverslips (Electron Microscopy Services) and fixed in fixation buffer (4% formaldehyde, 2% sucrose in PBS) for 20 min at RT. Cells were washed with PBS, permeabilized in permeabilization buffer (0.4% Triton X-100 and 0.4% IgG-free BSA) for 20 min at RT, washed with PBS again, and incubated with primary antibodies overnight at 4° C. The next day, after washing 3-5 times in the washing buffer (0.2% Triton X-100+0.2% IgG-free BSA in PBS), the cells were incubated with Alexa Fluor-conjugated secondary antibody (Invitrogen) for 2-3 h in the dark at RT. The coverslips were washed 3-5 times with washing buffer prior to mounting on glass slides using Vectashield Antifade Mounting Media (Vector Laboratories). Images were acquired using a Keyence BZ-X710 fluorescence microscope and analyzed using FIJI software (NIH).
(b) Immunofluorescent staining of human tissue microarray (TMA) and mouse BCBM tissue

Related to FIG. 16: Immunostaining for AAL, CAFs, and BC cells was performed on paraffin embedded FFPE tumor tissue sections (TMA slide, Serial #BR10010f; US BioMax). The slide was melted at 60° C. for 30 min, de-paraffinized using xylene and rehydrated in serial alcohol washes. The slide was pressure cooked at 15 PSI for 15 min in a 1×DAKO antigen retrieval buffer (Agilent Technologies (Santa Clara, CA)). The slide was then subjected to two 5-min standing washes in PBS prior to blocking in 1× Carb-Free Blocking Solution (Vector Labs (Burlingame, CA)) for 3 h at RT. The slide was next washed twice and incubated with anti-SMA, anti-FAP and AAL-488 antibodies O/N at 4° C. The next day, the slide was washed with PBS 3 times and incubated with secondary antibody anti-mouse-647 in dark for 3 h at RT. The slide was next washed and incubated with AAL-488 and eFluro-570 Anti-Pan Cytokeratin (Invitrogen) in dark for 3 h at RT. The slide was finally washed and mounted with Vectashield+DAPI (Vector Laboratories (Burlingame, CA)).

Related to FIG. 21 (mouse BCBM tissue): Immunostaining for injected human bmCAFs and MDA-MB-231bm cells was performed on the indicated mouse BCBM tumor sections. After the initial slide processing (as described above), the tissues were blocked in 6.5% IgG-free BSA (Jackson ImmunoResearch Laboratories) for 4 h at RT. The slides were next washed twice with PBS and incubated with anti-SMA-rabbit O/N at 4° C. The next day, tissue sections were washed three times with PBS and incubated with Anti-rabbit-488 and eFluro-570 Anti-Pan Cytokeratin for 3 h at RT. The slides were finally washed and mounted with Vectashield+DAPI (Vector Laboratories (Burlingame, CA)).

Related to FIG. 22 (total PVR levels in brain metastasis human tissue): Immunostaining for total-PVR, CAFs, and PanCK cells was performed on paraffin embedded FFPE tumor tissue sections. After the initial slide processing (as described above), the tissue was blocked in 6.5% IgG-free BSA (Jackson ImmunoResearch Laboratories) for 4 h at RT. The slide was next washed twice with PBS and incubated with anti-SMA, anti-FAP and anti-PVR antibodies O/N at 4° C. The next day, the slide was washed with PBS 3 times and incubated with secondary antibodies anti-mouse-488 and anti-rabbitt-647 in dark for 3 h at RT. The slide was next washed and incubated with eFluro 570 (Cy3) anti-PanCK in dark for 3 h at RT. The slide was finally washed and mounted with Vectashield+DAPI (Vector Laboratories (Burlingame, CA)).

Related to FIG. 22 (fuco-PVR levels in brain metastasis human tissue): Immunostaining for fuco-PVR, CAFs, and PanCK cells was performed on paraffin embedded FFPE tumor tissue sections. After the initial slide processing (as described above), the tissue was blocked in 6.5% IgG-free BSA (Jackson ImmunoResearch Laboratories) for 4 h at RT. The slide was next washed twice with PBS and incubated with anti-SMA, anti-FAP, anti-PVR, and biotinylated AAL antibodies O/N at 4° C. Next day, tissue sections were washed 3 times with PBS and incubated with donkey-mouse-488 and Goat-anti-biotin secondary antibodies for 3 h at RT. Next, the slides were proceeded for L-PLA as described below.
(c) Lectin-mediated proximity ligation assay (L-PLA)
   (i) L-PLA in tissue sections

L-PLA protocol was adapted from the manufacturer's protocols for the Duolink In Situ Far Red PLA kit (MilliporeSigma). PLA anti-goat PLUS and PLA anti-rabbit MINUS probes were applied at 1:5 for 1 h at 37° C. The slides were washed twice with wash buffer A before ligation with 1:5 ligation buffer and 1:40 ligase in ddH₂O for 30 min at 37° C. Slides were washed twice with wash buffer A before incubation in amplification mix (1:5 amplification buffer and 1:80 polymerase in ddH₂O for 1.5 h at 37° C.). Slides were washed twice with wash buffer B before mounting with VECTASHIELD and DAPI.
(ii) L-PLA in coverslip-grown fibroblasts

Briefly, coverslip-grown NBFs, tCAFs, and bmCAFs were fixed, permeabilized, and blocked as described above. Coverslips were washed twice with PBS and anti-PVR and biotinylated AAL lectin antibodies were incubated overnight at 4° C. Next day, coverslips were washed twice with PBS and incubated in a humidified chamber with phalloidin Alexa Fluor 488 and goat anti-biotin antibody for 2 h at RT in dark. Coverslips were next proceeded for L-PLA reactions as described above.
(d) Breast and brain metastasis TMA image acquisition, processing, and analysis

The multiplex immunofluorescence TMA images were scanned and quantitatively analyzed by the Analytic Microscopy Core at Moffitt Cancer Center. Briefly, immunofluorescently stained TMA (BR10010f and GL861a) slides were imaged with a Zeiss Imager Z2 microscope and Zen software version 2.3 (Carl Zeiss AG, Germany) using a 20×/0.8 NA objective lens and Hamamatsu Flash 4.0 V3 CMOS camera (Hamamatsu, Japan). An X-Cite Xylis broad spectrum LED light source (Excellitas Technologies Corp., Canada) and DAPI, FITC, dsRed, and CY5 filter cubes were used to excite and capture emissions of each fluorophore. Whole slide images were automatically captured using the tile scan mode with parabolic surface saddle autofocus on anchor points placed on each TMA tissue spot. The resulting images were stitched and background corrected using Zen software with background reference images for each fluorescence channel. The PLA stained GL861a slide was captured with 4 z-plates at 0.75 μm intervals and a maximum projection image was prepared from the stitched whole slide image.

The whole-slide TMA images (CZI format) were imported into Definiens Tissue Studio version 4.7 (Definiens AG, Germany), where individual cores were identified using the software's automated TMA segmentation tool. Each core was segmented into Tumor and Non-Tumor regions using a machine learning algorithm trained on the PCK channel. A small subset of cores required manual correction of the segmentation using the software's manual region of interest (ROI) drawing tool. Using these selected ROIs, a nuclear segmentation algorithm was applied to the DAPI channel to identify nuclei, and a cell growth algorithm was used to create individual cell boundaries. A minimum size threshold of 15 μm² was used to refine the nucleus and cell segmentations. Cell data features including mean fluorescence intensity (MFI) for the FITC and Cy5 channels were extracted for each cell within the BR10010f and GL861a datasets. Thresholds were applied to this data to determine the number of cells that express positive staining for AAL (BR10010f), PVR (GL861a), or SMA/FAP (BR10010f and GL861a) within the Tumor and Non-Tumor regions. For PLA slide analysis, the SPOT detection tool was applied on PLA stained GL861a TMA to identify foci using consistent intensity and size threshold settings across all TMA cores. Data for each TMA core was extracted to excel file format, including mean fluorescence intensity (MFI) values for the SMA/FAP channel and the number of PLA/L-PLA foci for the cell, cytoplasm, and nucleus compartments for both the Tumor and Non-Tumor regions.
(19) METABRIC dataset analysis

Clinical data of BC patients and PVR, FUT11, TJP1 microarray datasets were downloaded. Patients were stratified based on disease stage. Graph visualization comparing gene expression based on patient stages or co-expression of PVR and TJP1 was performed using Prism 9.
(20) GSEA analysis

Global mRNA expression profiles of the TCGA_Breast dataset were downloaded from Broad GDAC Firehose and subjected to Gene Set Enrichment Analysis (GSEA) to evaluate the association of protein of interest level with hallmark gene sets. For GSEA, the expression of PVR, FUT11, and phosphoproteomics hits (TJP1, EPHA2, PTPN12, AHNAK, and ARHGEF2) were applied as the phenotype label, and “No_Collapse” was used for the gene symbol. The metric for ranking genes was selected as ‘Pearson’. Analyses were performed using GSEA software (version 4.2.3).
(21) STRING and cytoscape network analysis

Protein-protein interaction (PPI) network analyses were performed. Textmining and Neighborhood were not included in the active interaction sources, the confidence level was set to high confidence (0.700), and max no. of interactions was set to 50. Cytoscape software (v3.9.1) was used to visualize the PP network from STRING interactors; curved style was chosen and only the direct interactors were represented.
(22) Quantification and statistical analysis

Statistical significance between the 2 groups was calculated using a two-tailed Student's t-test. Pearson correlation coefficient was used for correlation analyses. R Studio was used for processing RNA-seq data of fibroblasts. Venn diagrams were generated through Bio Venn web application. Data are presented as mean±standard error of the mean (SEM). P-values <0.005 were considered as statistically significant. All experiments were performed in at least 3 biologically independent replicates.

TABLE 1
Bacterial and virus strains

| | | |
|---|---|---|
| NEB® Stable Competent E. coli (High Efficiency) | New England | Cat#: C3040 |
| | Biolabs | |
| Human: PVR shRNA lentivirus; Sequence: | Sigma-Aldrich | Type: |
| CCGGTCTCCCAGTGGAGCCATATAGCTCGAGC | shRNA library (St | NM_006505.3- |
| TATATGGCTCCACTGGGAGATTTTTG (SEQ ID NO: | Louis, MO, USA) | 1884s21c1 |
| 1) | | |
| Human: PVR shRNA lentivirus; Sequence: | Sigma-Aldrich | Type: |
| CCGGTAGGCTCACAGTTACAGTTTACTCGAGT | shRNA library (St | NM_006505.3- |
| AAACTGTAACTGTGAGCCTATTTTTG (SEQ ID NO: | Louis, MO, USA) | 1771s21c1 |
| 2) | | |
| Human: PVR shRNA lentivirus; Sequence: | Sigma-Aldrich | Type: |
| CCGGGGATCGGGATTTATTTCTATTCTCGAGA | shRNA library (St | NM_006505.3- |
| ATAGAAATAAATCCCGATCCTTTTTG (SEQ ID NO: | Louis, MO, USA) | 1378s21c1 |
| 3) | | |
| Human: FUT11 shRNA lentivirus; Sequence: | Sigma-Aldrich | Type: |
| GCTGGCAGAGTTTATTGACTT (SEQ ID NO: 4) | shRNA library (St | NM_173540 |
| | Louis, MO, USA) | |
| Human: FUT11 shRNA lentivirus; Sequence: | Sigma-Aldrich | Type: |
| GCGGAGCTAAGGAGATCTTAT (SEQ ID NO: 5) | shRNA library (St | NM_173540 |
| | Louis, MO, USA) | |
| Human: FUT11 shRNA lentivirus; Sequence: | Sigma-Aldrich | Type: |
| CCTACATGAAATCTTCATGAA (SEQ ID NO: 6) | shRNA library (St | NM_173540 |
| | Louis, MO, USA) | |

Oligonucleotides

| | |
|---|---|
| Infusion-PVR-Forward: GAC GCG TAC GCG GCC ATG | Integrated DNA |
| GCC CGA GC (SEQ ID NO: 7) | Technologies (IDT) |
| Infusion-PVR-Reverse: TCT GCT CGA GCG GCC ACC | Integrated DNA |
| TTG TGC CCT (SEQ ID NO: 8) | Technologies (IDT) |
| V2 forward sequence: AGAGCTCGTTTAGTGAA (SEQ | Integrated DNA |
| ID NO: 9) | Technologies (IDT) |
| V2 Reverse sequence: | Integrated DNA |
| AGCAAATGATATCCTGGATTACAAGGA (SEQ ID | Technologies (IDT) |
| NO: 10) | |
| FUT1 Forward: GCAGCTTCACGACTGGATGT (SEQ | Integrated DNA |
| ID NO: 11) | Technologies (IDT) |
| FUT1 Reverse: CTCTCTGCGGAGCTGTTCCC (SEQ | Integrated DNA |
| ID NO: 12) | Technologies (IDT) |
| FUT4 Forward: CCGCTACTACCACCAACTGAG (SEQ | Integrated DNA |
| ID NO: 13) | Technologies (IDT) |
| FUT 4 Reverse: CTGCGAGTTCTCGAAAGCCA (SEQ | Integrated DNA |
| ID NO: 14) | Technologies (IDT) |
| FUT8 Forward: GGTTGCTGCTTTTGCTCAGA (SEQ ID | Integrated DNA |
| NO: 15) | Technologies (IDT) |
| FUT8 Reverse: CATGCGCTCTACATGCTGAAT (SEQ | Integrated DNA |
| ID NO: 16) | Technologies (IDT) |
| FUT10 Forward: TTTTGAGCAGCGCGAGAGTA (SEQ | Integrated DNA |
| ID NO: 17) | Technologies (IDT) |
| FUT10 Reverse: GCCCACAAACTGAATGCCTG (SEQ | Integrated DNA |
| ID NO: 18) | Technologies (IDT) |
| FUT11 Forward: AAGCTCTCACTGCCATGATCC | Integrated DNA |
| (SEQ ID NO: 19) | Technologies (IDT) |
| FUT11 Reverse: CTCCTTAGCTCCGCCAAGTT (SEQ | Integrated DNA |
| ID NO: 20) | Technologies (IDT) |
| H3A Forward: AAGCGAACTGCCCGCAAAT (SEQ ID | Integrated DNA |
| NO: 21) | Technologies (IDT) |
| H3A Reverse: GGCCTGTAACGATGAGGTTTC (SEQ | Integrated DNA |
| ID NO: 22) | Technologies (IDT) |
| FUT11 promoter positive for Chip qRT-PCR | Integrated DNA |
| Forward: AGCCTCATCTTTACTCCCGAAA (SEQ ID | Technologies (IDT) |
| NO: 23) | |
| FUT11 promoter positive for Chip qRT-PCR | Integrated DNA |
| Reverse: ATAGTCGTGACCGGACGAG (SEQ ID NO: | Technologies (IDT) |
| 24) | |
| FUT11 promoter negative for Chip qRT-PCR | Integrated DNA |
| Forward: GTGCACAAGAGCTGGGGTAT (SEQ ID | Technologies (IDT) |
| NO: 25) | |
| FUT11 promoter negative for Chip qRT-PCR | Integrated DNA |
| Reverse: GCAACTCAGTTCTCCGGTCA (SEQ ID NO: | Technologies (IDT) |
| 26) | |

### REFERENCES

Adhikari, E., Liu, Q., Burton, C., Mockabee-Macias, A., Lester, D. K., and Lau, E. (2022). L-fucose, a sugary regulator of antitumor immunity and immunotherapies. Mol. Carcinog. 61, 439-453.
Allinen, M., Beroukhim, R., Cai, L., Brennan, C., Lahti-Domenici, J., Huang, H., Porter, D., Hu, M., Chin, L., Richardson, A., et al. (2004). Molecular characterization of the tumor microenvironment in breast cancer. Cancer Cell 6, 17-32.
Bailleux, C., Eberst, L., and Bachelot, T. (2021). Treatment strategies for breast cancer brain metastases. Br. J. Cancer 124, 142-155.
Blanas, A., Sahasrabudhe, N. M., Rodríguez, E., van Kooyk, Y., and van Vliet, S. J. (2018). Fucosylated Antigens in Cancer: An Alliance toward Tumor Progression, Metastasis, and Resistance to Chemotherapy. Front. Oncol. 8, 39.
Boleij, A., Laarakkers, C. M., Gloerich, J., Swinkels, D. W., and Tjalsma, H. (2011). Surface-affinity profiling to identify host-pathogen interactions. Infect. Immun. 79, 4777-4783.
Bos, P. D., Zhang, X. H. F., Nadal, C., Shu, W., Gomis, R. R., Nguyen, D. X., Minn, A. J., van de Vijver, M. J., Gerald, W. L., Foekens, J. A., and Massague, J. (2009). Genes that mediate breast cancer metastasis to the brain. Nature 459, 1005-1009.
Bowers, J. R., Readler, J. M., Sharma, P., and Excoffon, K. J. D. A. (2017). Poliovirus Receptor: More than a simple viral receptor. Virus Res. 242, 1-6.
Buchsbaum, R. J., and Oh, S. Y. (2016). Breast Cancer-Associated Fibroblasts: Where We Are and Where We Need to Go. Cancers 8, 19.
Cagney, D. N., Martin, A. M., Catalano, P. J., Brown, P. D., Alexander, B. M., Lin, N. U., and Aizer, A. A. (2018). Implications of Screening for Brain Metastases in Patients With Breast Cancer and Non-Small Cell Lung Cancer. JAMA Oncol. 4, 1001-1003.
Cao, C., Backer, J. M., Laporte, J., Bedrick, E. J., and Wandinger-Ness, A. (2008). Sequential actions of myotubularin lipid phosphatases regulate endosomal PI(3)P and growth factor receptor trafficking. Mol. Biol. Cell 19, 3334-3346.
Cao, W., Zeng, Z., Pan, R., Wu, H., Zhang, X., Chen, H., Nie, Y., Yu, Z., and Lei, S. (2021). Hypoxia-Related Gene FUT11 Promotes Pancreatic Cancer Progression by Maintaining the Stability of PDK1. Front. Oncol. 11, 675991.
Chardin, P., and McCormick, F. (1999). Brefeldin A: the advantage of being uncompetitive. Cell 97, 153-155.
Chen, Y., McAndrews, K. M., and Kalluri, R. (2021). Clinical and therapeutic relevance of cancer-associated fibroblasts. Nat. Rev. Clin. Oncol. 18, 792-804.
Choi, Y. P., Lee, J. H., Gao, M. Q., Kim, B. G., Kang, S., Kim, S. H., and Cho, N. H. (2014). Cancer-associated fibroblast promote transmigration through endothelial brain cells in three-dimensional in vitro models. Int. J. Cancer 135, 2024-2033.
Chung, B., Esmaeili, A. A., Gopalakrishna-Pillai, S., Murad, J. P., Andersen, E. S., Kumar Reddy, N., Srinivasan, G., Armstrong, B., Chu, C., Kim, Y., et al. (2017). Human brain metastatic stroma attracts breast cancer cells via chemokines CXCL16 and CXCL12. NPJ Breast Cancer 3, 6.
Cohen, M. J., Chirico, W. J., and Lipke, P. N. (2020). Through the back door: Unconventional protein secretion. Cell Surf. 6, 100045.
Cruz-Muñoz, W., and Kerbel, R. S. (2011). Preclinical approaches to study the biology and treatment of brain metastases. Semin. Cancer Biol. 21, 123-130.
Curtis, C., Shah, S. P., Chin, S. F., Turashvili, G., Rueda, O. M., Dunning, M. J., Speed, D., Lynch, A. G., Samarajiwa, S., Yuan, Y., et al. (2012). The genomic and transcriptomic architecture of 2,000 breast tumours reveals novel subgroups. Nature 486, 346-352.
Duda, D. G., Duyverman, A. M. M. J., Kohno, M., Snuderl, M., Steller, E. J. A., Fukumura, D., and Jain, R. K. (2010). Malignant cells facilitate lung metastasis by bringing their own soil. Proc. Natl. Acad. Sci. USA 107, 21677-21682.
Ebright, R. Y., Zachariah, M. A., Micalizzi, D. S., Wittner, B. S., Niederhoffer, K. L., Nieman, L. T., Chirn, B., Wiley, D. F., Wesley, B., Shaw, B., et al. (2020). HIF1A signaling selectively supports proliferation of breast cancer in the brain. Nat. Commun. 11, 6311.
Erdogan, B., and Webb, D. J. (2017). Cancer-associated fibroblasts modulate growth factor signaling and extracellular matrix remodeling to regulate tumor metastasis. Biochem. Soc. Trans. 45, 229-236.
Fares, J., Fares, M. Y., Khachfe, H. H., Salhab, H. A., and Fares, Y. (2020). Molecular principles of metastasis: a hallmark of cancer revisited. Signal Transduct. Target. Ther. 5, 28.
Freeze, H. H., and Kranz, C. (2010). Endoglycosidase and glycoamidase release of N-linked glycans. Curr. Protoc. Protein Sci. Chapter 12, Unit 12.4
Gil-Gil, M. J., Martinez-Garcia, M., Sierra, A., Conesa, G., Del Barco, S., González-Jimenez, S., and Villa, S. (2014). Breast cancer brain metastases: a review of the literature and a current multidisciplinary management guideline. Clin. Transl. Oncol. 16, 436-446.
Glabman, R. A., Choyke, P. L., and Sato, N. (2022). Cancer-Associated Fibroblasts: Tumorigenicity and Targeting for Cancer Therapy. Cancers 14, 3906.
Gyorffy, B. (2021). Survival analysis across the entire transcriptome identifies biomarkers with the highest prognostic power in breast cancer. Comput. Struct. Biotechnol. J. 19, 4101-4109.
Hu, M., Yao, J., Carroll, D. K., Weremowicz, S., Chen, H., Carrasco, D., Richardson, A., Violette, S., Nikolskaya, T., Nikolsky, Y., et al. (2008). Regulation of in situ to invasive breast carcinoma transition. Cancer Cell 13, 394-406.
Hu, Y. L., Lu, S., Szeto, K. W., Sun, J., Wang, Y., Lasheras, J. C., and Chien, S. (2014). FAK and paxillin dynamics at focal adhesions in the protrusions of migrating cells. Sci. Rep. 4, 6024.
Huang, D. W., Sherman, B. T., and Lempicki, R. A. (2009). Systematic and integrative analysis of large gene lists using DAVID bioinformatics resources. Nat. Protoc. 4, 44-57.
Iguchi-Manaka, A., Okumura, G., Kojima, H., Cho, Y., Hirochika, R., Bando, H., Sato, T., Yoshikawa, H., Hara, H., Shibuya, A., and Shibuya, K. (2016). Increased Soluble CD155 in the Serum of Cancer Patients. PLoS One 11, e0152982.
Innocenti, M. (2018). New insights into the formation and the function of lamellipodia and ruffles in mesenchymal cell migration. Cell Adh. Migr. 12, 401-416.
Ju, L., Wang, Y., Xie, Q., Xu, X., Li, Y., Chen, Z., and Li, Y. (2016). Elevated level of serum glycoprotein bifucosylation and prognostic value in Chinese breast cancer. Glycobiology 26, 460-471.
Kakunaga, S., Ikeda, W., Shingai, T., Fujito, T., Yamada, A., Minami, Y., Imai, T., and Takai, Y. (2004). Enhancement of serum- and platelet-derived growth factor-induced cell proliferation by Necl-5/Tage4/poliovirus receptor/CD155 through the Ras-Raf-MEK-ERK signaling. J. Biol. Chem. 279, 36419-36425.
Karnoub, A. E., Dash, A. B., Vo, A. P., Sullivan, A., Brooks, M. W., Bell, G. W., Richardson, A. L., Polyak, K., Tubo, R., and Weinberg, R. A. (2007). Mesenchymal stem cells within tumour stroma promote breast cancer metastasis. Nature 449, 557-563.
Keeley, T. S., Yang, S., and Lau, E. (2019). The Diverse Contributions of Fucose Linkages in Cancer. Cancers 11, 1241.
Kennecke, H., Yerushalmi, R., Woods, R., Cheang, M. C. U., Voduc, D., Speers, C. H., Nielsen, T. O., and Gelmon, K. (2010). Metastatic behavior of breast cancer subtypes. J. Clin. Oncol. 28, 3271-3277.
Kim, J. S., and Kim, I. A. (2020). Evolving treatment strategies of brain metastases from breast cancer: current status and future direction. Ther. Adv. Med. Oncol. 12, 1758835920936117.
Knier, N. N., Pellizzari, S., Zhou, J., Foster, P. J., and Parsyan, A. (2022). Preclinical Models of Brain Metastases in Breast Cancer. Biomedicines 10, 667.
Krauss, M., and Haucke, V. (2007). Phosphoinositide-metabolizing enzymes at the interface between membrane traffic and cell signaling. EMBO Rep. 8, 241-246.
Kucan Brlic, P., Lenac Rovis, T., Cinamon, G., Tsukerman, P., Mandelboim, O., and Jonjic, S. (2019). Targeting PVR (CD155) and its receptors in anti-tumor therapy. Cell. Mol. Immunol. 16, 40-52.
Law, C. W., Chen, Y., Shi, W., and Smyth, G. K. (2014). voom: Precision weights unlock linear model analysis tools for RNA-seq read counts. Genome Biol. 15, R29.
LeBleu, V. S., and Neilson, E. G. (2020). Origin and functional heterogeneity of fibroblasts. FASEB J 34, 3519-3536.
Lee, H. W., Kim, Y., Han, K., Kim, H., and Kim, E. (2010). The phosphoinositide 3-phosphatase MTMR2 interacts with PSD-95 and maintains excitatory synapses by modulating endosomal traffic. J. Neurosci. 30, 5508-5518.
Lester, D. K., Burton, C., Gardner, A., Innamarato, P., Kodumudi, K., Liu, Q., Adhikari, E., Ming, Q., Williamson, D. B., Frederick, D. T., et al. (2023). Fucosylation of HLA-DRB1 regulates CD4(+) T cell-mediated anti-melanoma immunity and enhances immunotherapy efficacy. Nat. Cancer 4, 222-239.
Lindner, T., Loktev, A., Giesel, F., Kratochwil, C., Altmann, A., and Haberkorn, U. (2019). Targeting of activated fibroblasts for imaging and therapy. EJNMMI Radiopharm. Chem. 4, 16.
Manjula, S., Monteiro, F., Rao Aroor, A., Rao, S., Annaswamy, R., and Rao, A. (2010). Assessment of serum L-fucose in brain tumor cases. Ann. Indian Acad. Neurol. 13, 33-36.
Marth, J. D., and Grewal, P. K. (2008). Mammalian glycosylation in immunity. Nat. Rev. Immunol. 8, 874-887. Marusyk, A., Tabassum, D. P., Janiszewska, M., Place, A. E., Trinh, A., Rozhok, A. I., Pyne,
S., Guerriero, J. L., Shu, S., Ekram, M., et al. (2016). Spatial Proximity to Fibroblasts Impacts Molecular Features and Therapeutic Sensitivity of Breast Cancer Cells Influencing Clinical Outcomes. Cancer Res. 76, 6495-6506.
Matsumoto, K., Yokote, H., Arao, T., Maegawa, M., Tanaka, K., Fujita, Y., Shimizu, C., Hanafusa, T., Fujiwara, Y., and Nishio, K. (2008). N-Glycan fucosylation of epidermal growth factor receptor modulates receptor activity and sensitivity to epidermal growth factor receptor tyrosine kinase inhibitor. Cancer Sci. 99, 1611-1617.
Muchlinska, A., Nagel, A., Pope, da, M., Szade, J., Niemira, M., Zielinski, J., Skokowski, J., Bednarz-Knoll, N., and Zaczek, A. J. (2022). Alpha-smooth muscle actin-positive cancer-associated fibroblasts secreting osteopontin promote growth of luminal breast cancer. Cell. Mol. Biol. Lett. 27, 45.
Mustillo, A., Ayoub, J. P., Charpentier, D., Yelle, L., and Florescu, M. (2020). Prognosis in young women less than 40 years of age with brain metastasis from breast cancer. Curr. Oncol. 27, 39-45.
Neophytou, C., Boutsikos, P., and Papageorgis, P. (2018). Molecular Mechanisms and Emerging Therapeutic Targets of Triple-Negative Breast Cancer Metastasis. Front. Oncol. 8, 31.
Nickel, W., and Rabouille, C. (2009). Mechanisms of regulated unconventional protein secretion. Nat. Rev. Mol. Cell Biol. 10, 148-155.
Okumura, G., Iguchi-Manaka, A., Murata, R., Yamashita-Kanemaru, Y., Shibuya, A., and Shibuya, K. (2020). Tumor-derived soluble CD155 inhibits DNAM-1-mediated antitumor activity of natural killer cells. J. Exp. Med. 217, 1.
Pereira, B., Chin, S. F., Rueda, O. M., Vollan, H. K. M., Provenzano, E., Bardwell, H. A., Pugh, M., Jones, L., Russell, R., Sammut, S. J., et al. (2016). The somatic mutation profiles of 2,433 breast cancers refines their genomic and transcriptomic landscapes. Nat. Commun. 7, 11479.
Pieri, V., Gallotti, A. L., Drago, D., Cominelli, M., Pagano, I., Conti, V., Valtorta, S., Coliva, A., Lago, S., Michelatti, D., et al. (2023). Aberrant L-Fucose Accumulation and Increased Core Fucosylation Are Metabolic Liabilities in Mesenchymal Glioblastoma. Cancer Res. 83, 195-218.
Qi, J., Tripathi, M., Mishra, R., Sahgal, N., Fazli, L., Ettinger, S., Placzek, W. J., Claps, G., Chung, L. W. K., Bowtell, D., et al. (2013). The E3 ubiquitin ligase Siah2 contributes to castration-resistant prostate cancer by regulation of androgen receptor transcriptional activity. Cancer Cell 23, 332-346.
Rillahan, C. D., Antonopoulos, A., Lefort, C. T., Sonon, R., Azadi, P., Ley, K., Dell, A., Haslam, S. M., and Paulson, J. C. (2012). Global metabolic inhibitors of sialyl- and fucosyltransferases remodel the glycome. Nat. Chem. Biol. 8, 661-668.
Ritchie, M. E., Phipson, B., Wu, D., Hu, Y., Law, C. W., Shi, W., and Smyth, G. K. (2015). limma powers differential expression analyses for RNA sequencing and microarray studies. Nucleic Acids Res. 43, e47.
Robinson, M. D., and Oshlack, A. (2010). A scaling normalization method for differential expression analysis of RNA-seq data. Genome Biol. 11, R25.
Rosato, F. E., Seltzer, M., Mullen, J., and Rosato, E. F. (1971). Serum fucose in the diagnosis of breast cancer. Cancer 28, 1575-1579.
Ruan, W., Yang, Y., Yu, Q., Huang, T., Wang, Y., Hua, L., Zeng, Z., and Pan, R. (2021). FUT11 is a target gene of HIF1alpha that promotes the progression of hepatocellular carcinoma. Cell Biol. Int. 45, 2275-2286.
Sahai, E., Astsaturov, I., Cukierman, E., DeNardo, D. G., Egeblad, M., Evans, R. M., Fearon, D., Greten, F. R., Hingorani, S. R., Hunter, T., et al. (2020). A framework for advancing our understanding of cancer-associated fibroblasts. Nat. Rev. Cancer 20, 174-186.
Schjoldager, K. T., Narimatsu, Y., Joshi, H. J., and Clausen, H. (2020). Global view of human protein glycosylation pathways and functions. Nat. Rev. Mol. Cell Biol. 21, 729-749.
Shannon, P., Markiel, A., Ozier, O., Baliga, N. S., Wang, J. T., Ramage, D., Amin, N., Schwikowski, B., and Ideker, T. (2003). Cytoscape: a software environment for integrated models of biomolecular interaction networks. Genome Res. 13, 2498-2504.
Sherman, B. T., Hao, M., Qiu, J., Jiao, X., Baseler, M. W., Lane, H. C., Imamichi, T., and Chang, W. (2022). DAVID: a web server for functional enrichment analysis and functional annotation of gene lists (2021 update). Nucleic Acids Res. 50, W216-W221.
Sloan, K. E., Eustace, B. K., Stewart, J. K., Zehetmeier, C., Torella, C., Simeone, M., Roy, J. E., Unger, C., Louis, D. N., Ilag, L. L., and Jay, D. G. (2004). CD155/PVR plays a key role in cell motility during tumor cell invasion and migration. BMC Cancer 4, 73.
Sloan, K. E., Stewart, J. K., Treloar, A. F., Matthews, R. T., and Jay, D. G. (2005). CD155/PVR enhances glioma cell dispersal by regulating adhesion signaling and focal adhesion dynamics. Cancer Res. 65, 10930-10937.
Solanki, R. L., Ramdeo, I. N., and Sachdev, K. N. (1978). Serum protein bound fucose in diagnosis of breast malignancy. Indian J. Med. Res. 67, 786-791.
Steentoft, C., Vakhrushev, S. Y., Joshi, H. J., Kong, Y., Vester-Christensen, M. B., Schjoldager, K. T. B. G., Lavrsen, K., Dabelsteen, S., Pedersen, N. B., Marcos-Silva, L., et al. (2013). Precision mapping of the human O-GalNAc glycoproteome through SimpleCell technology. EMBO J. 32, 1478-1488.
Takai, K., Le, A., Weaver, V. M., and Werb, Z. (2016). Targeting the cancer associated fibroblasts as a treatment in triple-negative breast cancer. Oncotarget 7, 82889-82901.
Tan, K. P., Ho, M. Y., Cho, H. C., Yu, J., Hung, J. T., and Yu, A. L. T. (2016). Fucosylation of LAMP-1 and LAMP-2 by FUT1 correlates with lysosomal positioning and autophagic flux of breast cancer cells. Cell Death Dis. 7, e2347.
Thies, K. A., Hammer, A. M., Hildreth, B. E., 3rd, Steck, S. A., Spehar, J. M., Kladney, R. D., Geisler, J. A., Das, M., Russell, L. O., Bey, J. F., 4th., et al. (2021). Stromal Platelet-Derived Growth Factor Receptor-beta Signaling Promotes Breast Cancer Metastasis in the Brain. Cancer Res. 81, 606-618.
Veillon, L., Fakih, C., Abou-El-Hassan, H., Kobeissy, F., and Mechref, Y. (2018). Glycosylation Changes in Brain Cancer. ACS Chem. Neurosci. 9, 51-72.
Waalkes, T. P., Mrochek, J. E., Dinsmore, S. R., and Tormey, D. C. (1978). Serum protein-bound carbohydrates for following the course of disease in patients with metastatic breast carcinoma. J. Natl. Cancer Inst. 61, 703-707.
Watase, C., Shiino, S., Shimoi, T., Noguchi, E., Kaneda, T., Yamamoto, Y., Yonemori, K., Takayama, S., and Suto, A. (2021). Breast Cancer Brain Metastasis-Overview of Disease State, Treatment Options and Future Perspectives. Cancers 13, 1078.
Watson, G., Lester, D., Ren, H., Forsyth, C. M., Medina, E., Gonzalez Perez, D., Darville, L., Yao, J., Luca, V., Koomen, J., et al. (2021). Fucosylated Proteome Profiling Identifies a Fucosylated, Non-Ribosomal, Stress Responsive Species of Ribosomal Protein S3. Cells 10, 1310.
Witzel, I., Oliveira-Ferrer, L., Pantel, K., Muller, V., and Wikman, H. (2016). Breast cancer brain metastases: biology and new clinical perspectives. Breast Cancer Res. 18, 8.
Yamaguchi, H., and Condeelis, J. (2007). Regulation of the actin cytoskeleton in cancer cell migration and invasion. Biochim. Biophys. Acta 1773, 642-652.
Yamashita, M., Ogawa, T., Zhang, X., Hanamura, N., Kashikura, Y., Takamura, M., Yoneda, M., and Shiraishi, T. (2012). Role of stromal myofibroblasts in invasive breast cancer: stromal expression of alpha-smooth muscle actin correlates with worse clinical outcome. Breast Cancer 19, 170-176.
Yazhou, C., Wenly, S., Weidong, Z., and Licun, W. (2004). Clinicopathological significance of stromal myofibroblasts in invasive ductal carcinoma of the breast. Tumour Biol. 25, 290-295.
Yuan, K., Kucik, D., Singh, R. K., Listinsky, C. M., Listinsky, J. J., and Siegal, G. P. (2008). Alterations in human breast cancer adhesion-motility in response to changes in cell surface glycoproteins displaying alpha-Lfucose moieties. Int. J. Oncol. 32, 797-807.
Yuan, K., Listinsky, C. M., Singh, R. K., Listinsky, J. J., and Siegal, G. P. (2008). Cell surface associated alpha-L-fucose moieties modulate human breast cancer neoplastic progression. Pathol. Oncol. Res. 14, 145-156.
Zhang, M., Liu, L., Lin, X., Wang, Y., Li, Y., Guo, Q., Li, S., Sun, Y., Tao, X., Zhang, D., et al. (2020). A Translocation Pathway for Vesicle-Mediated Unconventional Protein Secretion. Cell 181, 637-652.e15.
Zhang, P., Haryadi, R., Chan, K. F., Teo, G., Goh, J., Pereira, N. A., Feng, H., and Song, Z. (2012). Identification of functional elements of the GDP fucose transporter SLC35C1 using a novel Chinese hamster ovary mutant. Glycobiology 22, 897-911.
Zhou, B., Moodie, A., Blanchard, A. A. A., Leygue, E., and Myal, Y. (2015). Claudin 1 in Breast Cancer: New Insights. J. Clin. Med. 4, 1960-1976.

1. A method of treating a cancer and/or metastasis in a subject comprising administering to the subject an agent that blocks polio virus receptor (PVR) and an agent that increases fucosylation.

2. The method of claim 1, wherein the agent that blocks polio virus receptor comprises an antibody, immunotoxin, small molecule, peptide, polypeptide, protein, antisense oligonucleotide, or siRNA.

3. The method of claim 1, wherein the agent that increases fucosylation comprises L-fucose, D-fucose, fucose-1-phosphate, or GDP-L-fucose.

4. The method of claim 1, t further comprising the administration of an anti-cancer agent.

5. The method of claim 4, wherein the anti-cancer agent comprises an immune checkpoint blockade inhibitor.

6. The method of t claim 5, wherein the immune checkpoint blockade inhibitor is a PD-1 inhibitors.

7. The method of claim 6, wherein the PD-1 inhibitor is a selected from the group consisting of lambrolizumab, nivolumab, BMS-936558, MDX1106, pembrolizumab, pidilizumab, cemiplimab, CT-011, and MK-3475.

8. The method of claim 5, wherein the immune checkpoint blockade inhibitor is a PD-L1 inhibitor.

9. The method of claim 8, wherein the PD-L1 inhibitor is a selected from the group consisting of atezolizumab, avelumab, durvalumab, MDX-1105, BMS-936559, MPDL3280 Å, and MSB0010718C.

10. The method of claim 5, wherein the immune checkpoint blockade inhibitor is a CTLA-4 inhibitor.

11. The method of claim 10, wherein the CTLA-4 inhibitor is a selected from the group consisting of ipilimumab and tremelimumab.

12. The method of claim 1, further comprising administering to the subject an adoptive cell therapy.

13. The method of claim 12, wherein the adoptive cell therapy comprises the transfer of tumor infiltrating lymphocytes (TILs), tumor infiltrating NK cells (TINKs), marrow infiltrating lymphocytes (MILs), chimeric antigen receptor (CAR) T cells, and/or CAR NK cells.

14. The method of claim 1, wherein the cancer and/or metastasis comprises breast cancer, triple negative breast cancer, and/or breast cancer brain metastasis.

15. A method of detecting the presence of a brain metastasis or measuring the progression of a brain metastasis in a subject with breast cancer, comprising:
a) obtaining a tissue sample from a subject, and
b) measuring the amount of secreted fucosylated PVR in the sample, wherein the presence of or an increase in fucosylated PVR relative to a control indicates the presence of a brain metastasis.

16. A method of treating a cancer and/or metastasis in a subject, comprising:
a) obtaining a tissue sample from a subject;
b) measuring the amount of secreted fucosylated PVR in the sample, wherein the presence of or an increase in fucosylated PVR relative to a control indicates the presence of a brain metastasis; and
c) administering to the subject an agent that blocks polio virus receptor and an agent that increases fucosylation when the presence of or an increase in fucosylated PVR is detected.
